SEQUENCE LISTING (124 sequences)

SEQ ID NO: 1 - protein, 8 aa, synthetic construct
  REGION 1..8: note = synthetic
GFTFSTYA

SEQ ID NO: 2 - protein, 10 aa, synthetic construct
  REGION 1..10: note = synthetic
IRSKYNNYAT

SEQ ID NO: 3 - protein, 16 aa, synthetic construct
  REGION 1..16: note = synthetic
VRHGNFGNSYVSWFAY

SEQ ID NO: 4 - protein, 125 aa, synthetic construct
  REGION 1..125: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 5 - protein, 9 aa, synthetic construct
  REGION 1..9: note = synthetic
TGAVTTSNY

SEQ ID NO: 6 - skipped

SEQ ID NO: 7 - protein, 9 aa, synthetic construct
  REGION 1..9: note = synthetic
ALWYSNLWV

SEQ ID NO: 8 - protein, 109 aa, synthetic construct
  REGION 1..109: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVL

SEQ ID NO: 9 - protein, 8 aa, synthetic construct
  REGION 1..8: note = synthetic
GFNIKDTY

SEQ ID NO: 10 - protein, 8 aa, synthetic construct
  REGION 1..8: note = synthetic
IYPTNGYT

SEQ ID NO: 11 - protein, 13 aa, synthetic construct
  REGION 1..13: note = synthetic
SRWGGDGFYAMDY

SEQ ID NO: 12 - protein, 120 aa, synthetic construct [sequence omitted]

SEQ ID NO: 13 - protein, 6 aa, synthetic construct
  REGION 1..6: note = synthetic
QDVNTA

SEQ ID NO: 14 - skipped

SEQ ID NO: 15 - protein, 9 aa, synthetic construct
  REGION 1..9: note = synthetic
QQHYTTPPT

SEQ ID NO: 16 - protein, 107 aa, synthetic construct [sequence omitted]

SEQ ID NO: 17 - protein, 8 aa, synthetic construct
  REGION 1..8: note = synthetic
GFTFTDYT

SEQ ID NO: 18 - protein, 8 aa, synthetic construct
  REGION 1..8: note = synthetic
VNPNSGGS

SEQ ID NO: 19 - protein, 12 aa, synthetic construct
  REGION 1..12: note = synthetic
ARNLGPSFYFDY

SEQ ID NO: 20 - protein, 119 aa, synthetic construct
  REGION 1..119: note = synthetic
EVQLVESGGGLVQPGGSLRLSCAASGFTFTDYTMDWVRQAPGKGAEWVADVNPNSGGSIYNQRFKGRFTLSVDRSKNTLYLQMNSLRAEDTAVYYCARNLGPSFYFDYWGQGTLVTVSS

SEQ ID NO: 21 - protein, 6 aa, synthetic construct
  REGION 1..6: note = synthetic
QDVSIG

SEQ ID NO: 22 - skipped

SEQ ID NO: 23 - protein, 9 aa, synthetic construct
  REGION 1..9: note = synthetic
QQYYIYPYT

SEQ ID NO: 24 - protein, 107 aa, synthetic construct
  REGION 1..107: note = synthetic
DIQMTQSPSSLSASVGDRVTITCKASQDVSIGVAWYQQKPGKAPKLLIYSASYRYTGVPSRFSGSGSGTDFTLTISSLQPEDFATYWCQQYYIYPYTFGQGTKVEIK

SEQ ID NO: 25 - protein, 8 aa, synthetic construct
  REGION 1..8: note = synthetic
GFTFSSYS

SEQ ID NO: 26 - protein, 8 aa, synthetic construct
  REGION 1..8: note = synthetic
ISSSSSYI

SEQ ID NO: 27 - protein, 15 aa, synthetic construct
  REGION 1..15: note = synthetic
ARGAPMGAAAGWFDP

SEQ ID NO: 28 - protein, 122 aa, synthetic construct
  REGION 1..122: note = synthetic
EVQLVESGGGLVKPGGSLRLSCAASGFTFSSYSMNWVRDAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 29 - protein, 6 aa, synthetic construct
  REGION 1..6: note = synthetic
QGISSW

SEQ ID NO: 30 - skipped

SEQ ID NO: 31 - protein, 9 aa, synthetic construct
  REGION 1..9: note = synthetic
QQGVSFPRT

SEQ ID NO: 32 - protein, 107 aa, synthetic construct
  REGION 1..107: note = synthetic
DIQMTQSPSSVSASVGDRVTITCRASQGISSWLAWYQKKPGKAPKLLIYAASSLQSGVPSRFSGSGSGTDFTLTISSLQPEDFATYYCQQGVSFPRTFGGGTKVEIK

SEQ ID NO: 33 - protein, 8 aa, synthetic construct
  REGION 1..8: note = synthetic
GYTFTSYY

SEQ ID NO: 34 - protein, 8 aa, synthetic construct
  REGION 1..8: note = synthetic
INPSGGST

SEQ ID NO: 35 - protein, 13 aa, synthetic construct
  REGION 1..13: note = synthetic
ARGSAYYYDFADY

SEQ ID NO: 36 - protein, 120 aa, synthetic construct
  REGION 1..120: note = synthetic
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 37 - protein, 6 aa, synthetic construct
  REGION 1..6: note = synthetic
NIGSKN

SEQ ID NO: 38 - skipped

SEQ ID NO: 39 - protein, 9 aa, synthetic construct
  REGION 1..9: note = synthetic
QVWDNYSVL

SEQ ID NO: 40 - protein, 106 aa, synthetic construct
  REGION 1..106: note = synthetic
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 41 - protein, 103 aa, synthetic construct
  REGION 1..103: note = synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - protein, 107 aa, synthetic construct [sequence omitted]

SEQ ID NO: 43 - protein, 15 aa, synthetic construct
  REGION 1..15: note = synthetic
DKTHTCPPCPAPPVA

SEQ ID NO: 44 - protein, 209 aa, synthetic construct
  REGION 1..209: note = synthetic
GPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 45 - protein, 209 aa, synthetic construct
  REGION 1..209: note = synthetic
GPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 46 - protein, 237 aa, synthetic construct
  REGION 1..237: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAEVQLVESGGGLVQPGGSLRLSCAASGFTFSTYAMNWVRDAPGKGLEWVGRIRSKYNNYATYYADSVKGRFTISRDDSKNSLYLQMNSLRAEDTAVYYCVRHGNFGNSYVTWFAYWGQGTLVTVSS

SEQ ID NO: 47 - protein, 465 aa, synthetic construct
  REGION 1..465: note = synthetic
QAVVTQEPSLTVSPGGTVTLTCRSSTGAVTTSNYANWVQKKPGQAPRGLIGGTNKRAPWTPARFSGSLLGGKAALTLSGAQAEDEADYYCALWYSNLWVFGGGTKLTVLRTVAAEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSSASEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 446 aa, synthetic construct
  REGION 1..446: note = synthetic
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 49 - protein, 214 aa, synthetic construct
  REGION 1..214: note = synthetic
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 234 aa, synthetic construct
  REGION 1..234: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAEVQLVESGGGLVKPGGSLRLSCAASGFTFSSYSMNWVRDAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 51 - protein, 464 aa, synthetic construct
  REGION 1..464: note = synthetic
DIQMTQSPSSVSASVGDRVTITCRASQGISSWLAWYQKKPGKAPKLLIYAASSLQSGVPSRFSGSGSGTDFTLTISSLQPEDFATYYCQQGVSFPRTFGGGTKVEIKRTVAAEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSSASEPKSSDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 447 aa, synthetic construct
  REGION 1..447: note = synthetic
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 53 - protein, 214 aa, synthetic construct; identical to SEQ ID NO: 49
  REGION 1..214: note = synthetic

SEQ ID NO: 54 - protein, 230 aa, synthetic construct
  REGION 1..230: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAEVQLVESGGGVVQPGGSLRLSCAASGFTFSSYGMHWVRDAPGKGLEWVAFIRYDGSNKYYADSVKGRFTISRDNSKNTKYLQMNSLRAEDTAVYYCAKDRFGYYLDYWGQGTLVTVSS

SEQ ID NO: 55 - protein, 465 aa, synthetic construct
  REGION 1..465: note = synthetic
QPVLTQPSSVSVAPGETARIPCGGDDIETKSVHWYQKKPGQAPVLVIYDDDDRPSGIPERFFGSNSGNTATLSISRVEAGDEADYYCQVWDDNNDEWVFGGGTQLTVLRTVAAEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSSASEPKSSDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 447 aa, synthetic construct; identical to SEQ ID NO: 52
  REGION 1..447: note = synthetic

SEQ ID NO: 57 - protein, 214 aa, synthetic construct; identical to SEQ ID NO: 49
  REGION 1..214: note = synthetic

SEQ ID NO: 58 - protein, 234 aa, synthetic construct; identical to SEQ ID NO: 50
  REGION 1..234: note = synthetic

SEQ ID NO: 59 - protein, 464 aa, synthetic construct; identical to SEQ ID NO: 51
  REGION 1..464: note = synthetic

SEQ ID NO: 60 - protein, 447 aa, synthetic construct
  REGION 1..447: note = synthetic
EVQLVESGGGLVQPGGSLRLSCAASGFTFTDYTMDWVRQAPGKGAEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 61 - protein, 214 aa, synthetic construct
  REGION 1..214: note = synthetic
DIQMTQSPSSLSASVGDRVTITCKASQDVSIGVAWYQQKPGKAPKLLIYSASYRYTGVPSRFSGSGSGTDFTLTISSLQPEDFATYWCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 237 aa, synthetic construct
  REGION 1..237: note = synthetic
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGCGTKVEIKRTVAAEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 63 - protein, 457 aa, synthetic construct
  REGION 1..457: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAEVQLVESGGGLVQPGGSLRLSCAASGFTFTDYTMDWVRKAPGKCLEWVADVNPNSGGSIYNQRFKGRFTLSVDRSKNTLYLQMNSLRAEDTAVYYCARNLGPSFYFDYWGQGTLVTVSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 64 - protein, 447 aa, synthetic construct
  REGION 1..447: note = synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 65 - protein, 214 aa, synthetic construct
  REGION 1..214: note = synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 232 aa, synthetic construct
  REGION 1..232: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAQVQLVQSGAEVKKPGESLKVSCKASGYTFTSYYMHWVRDAPGQGLEWMGIINPSGGSTSYAQKFQGRVTMTRDTSTSTVYMELSSLRSEDTAVYYCARGSAYYYDFADYWGQGTLVTVSS

SEQ ID NO: 67 - protein, 462 aa, synthetic construct
  REGION 1..462: note = synthetic
SYVLTQPSSVSVAPGQTATISCGGHNIGSKNVHWYQKRPGQSPVLVIYQDNKRPSGIPERFSGSNSGNTATLTISGTQAMDEADYYCQVWDNYSVLFGGGTKLTVLRTVAAEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSSASEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 68 - protein, 447 aa, synthetic construct; identical to SEQ ID NO: 60
  REGION 1..447: note = synthetic

SEQ ID NO: 69 - protein, 214 aa, synthetic construct; identical to SEQ ID NO: 61
  REGION 1..214: note = synthetic

SEQ ID NO: 70 - protein, 234 aa, synthetic construct
  REGION 1..234: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSS

SEQ ID NO: 71 - protein, 468 aa, synthetic construct
  REGION 1..468: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 72 - protein, 446 aa, synthetic construct; identical to SEQ ID NO: 48
  REGION 1..446: note = synthetic

SEQ ID NO: 73 - protein, 214 aa, synthetic construct; identical to SEQ ID NO: 49
  REGION 1..214: note = synthetic

SEQ ID NO: 74 - protein, 232 aa, synthetic construct
  REGION 1..232: note = synthetic
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGCGTKVEIKRTVAAQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 75 - protein, 461 aa, synthetic construct
  REGION 1..461: note = synthetic
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLRTVAAEVQLVESGGGLVQPGGSLRLSCAASGFTFTDYTMDWVRKAPGKCLEWVADVNPNSGGSIYNQRFKGRFTLSVDRSKNTLYLQMNSLRAEDTAVYYCARNLGPSFYFDYWGQGTLVTVSSASEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 447 aa, synthetic construct; identical to SEQ ID NO: 64
  REGION 1..447: note = synthetic

SEQ ID NO: 77 - protein, 214 aa, synthetic construct; identical to SEQ ID NO: 65
  REGION 1..214: note = synthetic

SEQ ID NO: 78 - protein, 237 aa, synthetic construct
  REGION 1..237: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQAYTTPPTFGCGTKVEIKRTVAAEVQLVESGGGLVQPGGSLRLSCAASGFTFSTYAMNWVRDAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 79 - protein, 465 aa, synthetic construct; identical to SEQ ID NO: 47
  REGION 1..465: note = synthetic

SEQ ID NO: 80 - protein, 447 aa, synthetic construct; identical to SEQ ID NO: 64
  REGION 1..447: note = synthetic

SEQ ID NO: 81 - protein, 214 aa, synthetic construct
  REGION 1..214: note = synthetic
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 237 aa, synthetic construct
  REGION 1..237: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKRTVAAEVQLVESGGGLVQPGGSLRLSCAASGFTFSTYAMNWVRDAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 83 - protein, 465 aa, synthetic construct; identical to SEQ ID NO: 47
  REGION 1..465: note = synthetic

SEQ ID NO: 84 - protein, 447 aa, synthetic construct; identical to SEQ ID NO: 64
  REGION 1..447: note = synthetic

SEQ ID NO: 85 - protein, 214 aa, synthetic construct; identical to SEQ ID NO: 65
  REGION 1..214: note = synthetic

SEQ ID NO: 86 - protein, 219 aa, synthetic construct
  REGION 1..219: note = synthetic
DIVLTQSPSSLAVSAGEKVTMSCKSSQSLLNSRTRKNYLTWYQQKPGQSPKLLIYWASTRDSGVPDRFSGSGSGTDFTLTISSVQAEDLAVYYCTQSYTLRTFGGGTKLEIKRTVAAPSVDIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGES

SEQ ID NO: 87 - protein, 446 aa, synthetic construct
  REGION 1..446: note = synthetic
QVQLQQSGPELVKPGTSVKMSCKASGYTFTNYYIHWVRQRPGQGLEWIGWIYPGDVSTKYNEKFKGKTTLTADKSSSTAYMLLSGLTSEDSAIYFCARDGYSFYYFDYWGQGTTLTVSSASTKGPSVFPLAPSSKSTSGGTAKLGCLVKDYFPEPVTVSWNSGALTSGVHTFPACLQSSGLYSLSSVVTVPSSSLGTQTYICNVNHKPSNTKVDKKVEPKSSDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSP

SEQ ID NO: 88 - protein, 447 aa, synthetic construct
  REGION 1..447: note = synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSP

SEQ ID NO: 89 - protein, 214 aa, synthetic construct; identical to SEQ ID NO: 65
  REGION 1..214: note = synthetic

SEQ ID NO: 90 - protein, 120 aa, synthetic construct
  REGION 1..120: note = synthetic
EVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSS

SEQ ID NO: 91 - protein, 107 aa, synthetic construct
  REGION 1..107: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIK

SEQ ID NO: 92 - protein, 119 aa, synthetic construct
  REGION 1..119: note = synthetic
EVQLVESGGGLVQPGGSLRLSCAASGFTFTDYTMDWVRKAPGKCLEWVADVNPNSGGSIYNQRFKGRFTLSVDRSKNTLYLQMNSLRAEDTAVYYCARNLGPSFYFDYWGQGTLVTVSS

SEQ ID NO: 93 - protein, 107 aa, synthetic construct
  REGION 1..107: note = synthetic
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGCGTKVEIK

SEQ ID NO: 94 - protein, 5 aa, synthetic construct
  REGION 1..5: note = synthetic
RTVAA

SEQ ID NO: 95 - protein, 107 aa, synthetic construct
  REGION 1..107: note = synthetic
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIK

SEQ ID NO: 96 - protein, 16 aa, synthetic construct
  REGION 1..16: note = synthetic
VRHGNFGNSYVTWFAY

SEQ ID NO: 97 - protein, 9 aa, synthetic construct
  REGION 1..9: note = synthetic
QQAYTTPPT

SEQ ID NO: 98 - protein, 107 aa, synthetic construct
  REGION 1..107: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQAYTTPPTFGCGTKVEIK

SEQ ID NO: 99 - protein, 125 aa, synthetic construct
  REGION 1..125: note = synthetic
EVQLVESGGGLVQPGGSLRLSCAASGFTFSTYAMNWVRDAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 100 - protein, 109 aa, synthetic construct
  REGION 1..109: note = synthetic
QAVVTQEPSLTVSPGGTVTLTCRSSTGAVTTSNYANWVQKKPGQAPRGLIGGTNKRAPWTPARFSGSLLGGKAALTLSGAQAEDEADYYCALWYSNLWVFGGGTKLTVL

SEQ ID NO: 101 - protein, 23 aa, synthetic construct
  REGION 1..23: note = synthetic
ASEPKSSDKTHTCPPCPAPELLA

SEQ ID NO: 102 - protein, 16 aa, synthetic construct
  REGION 1..16: note = synthetic
DKTHTCPPCPAPELLA

SEQ ID NO: 103 - protein, 209 aa, synthetic construct
  REGION 1..209: note = synthetic
GPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 104 - protein, 209 aa, synthetic construct
  REGION 1..209: note = synthetic
GPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 105 - protein, 125 aa, synthetic construct
  REGION 1..125: note = synthetic
EVQLVESGGGLVQPGGSLRLSCAASGFTFSTYAMNWVRDAPGKGLEWVGRIRSKYNNYATYYADSVKGRFTISRDDSKNSLYLQMNSLRAEDTAVYYCVRHGNFGNSYVTWFAYWGQGTLVTVSS

SEQ ID NO: 106 - protein, 119 aa, synthetic construct [sequence omitted]

SEQ ID NO: 107 - protein, 107 aa, synthetic construct [sequence omitted]

SEQ ID NO: 108 - protein, 120 aa, synthetic construct
  REGION 1..120: note = synthetic
QVQLVQSGAEVKKPGESLKVSCKASGYTFTSYYMHWVRDAPGQGLEWMGIINPSGGSTSYAQKFQGRVTMTRDTSTSTVYMELSSLRSEDTAVYYCARGSAYYYDFADYWGQGTLVTVSS

SEQ ID NO: 109 - protein, 106 aa, synthetic construct
  REGION 1..106: note = synthetic
SYVLTQPSSVSVAPGQTATISCGGHNIGSKNVHWYQKRPGQSPVLVIYQDNKRPSGIPERFSGSNSGNTATLTISGTQAMDEADYYCQVWDNYSVLFGGGTKLTVL

SEQ ID NO: 110 - protein, 235 aa, synthetic construct
  REGION 1..235: note = synthetic
DIQMTQSPSSLSASVGDRVTITCRASQDVNTAVAWYQDKPGKAPKLLIYSASFLYSGVPSRFSGSRSGTDFTLTISSLQPEDFATYYCQQHYTTPPTFGCGTKVEIKGGSGGSGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 111 - protein, 468 aa, synthetic construct
  REGION 1..468: note = synthetic
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGGSGGSGGSEVQLVESGGGLVQPGGSLRLSCAASGFNIKDTYIHWVRKAPGKCLEWVARIYPTNGYTRYADSVKGRFTISADTSKNTAYLQMNSLRAEDTAVYYCSRWGGDGFYAMDYWGQGTLVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 112 - protein, 236 aa, synthetic construct
  REGION 1..236: note = synthetic
DIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGCGTKVEIKGGSGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 113 - protein, 467 aa, synthetic construct
  REGION 1..467: note = synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGSGGSEVQLVESGGGLVQPGGSLRLSCAASGFTFTDYTMDWVRKAPGKCLEWVADVNPNSGGSIYNQRFKGRFTLSVDRSKNTLYLQMNSLRAEDTAVYYCARNLGPSFYFDYWGQGTLVTVSSASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 114 - protein, 8 aa, synthetic construct
  REGION 1..8: note = synthetic
FTFSYGMH

SEQ ID NO: 115 - protein, 17 aa, synthetic construct
  REGION 1..17: note = synthetic
FIRYDGSNKYYADSVKG

SEQ ID NO: 116 - protein, 11 aa, synthetic construct
  REGION 1..11: note = synthetic
AKDRFGYYLDY

SEQ ID NO: 117 - protein, 118 aa, synthetic construct
  REGION 1..118: note = synthetic
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 118 - protein, 11 aa, synthetic construct
  REGION 1..11: note = synthetic
GGDDIETKSVH

SEQ ID NO: 119 - protein, 7 aa, synthetic construct
  REGION 1..7: note = synthetic
DDDDRPS

SEQ ID NO: 120 - protein, 11 aa, synthetic construct
  REGION 1..11: note = synthetic
QVWDDNNDEWV

SEQ ID NO: 121 - protein, 108 aa, synthetic construct
  REGION 1..108: note = synthetic
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 122 - protein, 118 aa, synthetic construct
  REGION 1..118: note = synthetic
EVQLVESGGGVVQPGGSLRLSCAASGFTFSSYGMHWVRDAPGKGLEWVAFIRYDGSNKYYADSVKGRFTISRDNSKNTKYLQMNSLRAEDTAVYYCAKDRFGYYLDYWGQGTLVTVSS

SEQ ID NO: 123 - protein, 108 aa, synthetic construct
  REGION 1..108: note = synthetic
QPVLTQPSSVSVAPGETARIPCGGDDIETKSVHWYQKKPGQAPVLVIYDDDDRPSGIPERFFGSNSGNTATLSISRVEAGDEADYYCQVWDDNNDEWVFGGGTQLTVL

SEQ ID NO: 124 - protein, 125 aa, synthetic construct
  REGION 1..125: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVTWFAYWGQGTLVTVSS

(19) United States
(12) Patent Application Publication
(10) Pub. No.: US 2026/0250395 A1
(43) Pub. Date: Aug. 27, 2026

(54) MULTI-SPECIFIC ANTIBODIES TARGETING A DIMERIZABLE TUMOR ANTIGEN AND AN IMMUNOSTIMULATORY ANTIGEN

(71) Applicant: CHIMAGEN BIOSCIENCES, LTD, Shanghai (CN)

(72) Inventors: Zhenhao ZHOU, Shanghai (CN); Jie ZHANG, Shanghai (CN); Xiaoqing WANG, Shanghai (CN); Gong WANG, Shanghai (CN)

(21) Appl. No.: 18/998,130

(22) PCT Filed: Aug. 4, 2023
(86) PCT No.: PCT/CN2023/111265

§ 371 Date: Jan. 24, 2025

(30) Foreign Application Priority Data
Aug. 5, 2022 (WO) PCT/CN2022/110698

(51) Int. Cl. C07K 16/28; A61K 39/00; A61K 45/06; A61P 35/00; C07K 16/32

(52) U.S. Cl. CPC C07K 16/2851; A61K 45/06; A61P 35/00; C07K 16/2809; C07K 16/283; C07K 16/32; A61K 2039/505; C07K 2317/31; C07K 2317/522; C07K 2317/524; C07K 2317/526; C07K 2317/53; C07K 2317/565; C07K 2317/624

(57) ABSTRACT

The present disclosure provides multi-specific antibodies targeting a dimerizable tumor antigen and an immunostimulatory antigen.

### FIELD OF THE INVENTION

The present invention relates to novel covalent multi-specific antibodies targeting a tumor antigen and an immunostimulatory antigen, and uses thereof.

### BACKGROUND

Multi-functional antibodies are constructed based on traditional antibodies through sophisticated design and molecular engineering, which enable the antibodies to bind to more than one antigen. A number of bi-specific antibody technology platforms have been developed, to name a few among them: BiTE Bi-specific T-cell Engaging (Micromet, acquired by Amgen in 2012), CrossMab (Roche), DVD-Ig (Abbvie), TandAb (Affimed), DART (Dual Antigen Re-Targeting, Macrogenics).

Practically, a bi-specific or multi-specific antibody is capable of delivering therapeutic effects same as that from a combination of several conventional antibodies. By simultaneous engagement of multiple targets of interest, the bi-specific or multi-specific antibody can deliver benefits superior than classic antibodies via novel and unique mechanisms. For example, Blinatumomab (CD3×CD19, Amgen) that targets CD3 and CD19 can efficiently engage T cells in the killing of CD19-expressing tumor cells via its CD3-recognizing Fv and showed superior efficacy over conventional antibodies in treating ALL (acute lymphoid leukemia) etc. Blinatumomab was approved to launch for ALL treatment by FDA in 2014.

However, despite of the advantages conferred by bi-specific or multi-specific antibodies, they also introduce challenges such as adverse effects, low response rate and limited effectiveness. In particular, certain targets are prone to dimerization or aggregation upon binding to a bi-specific or multi-specific antibody, which could lead to unwanted adverse effects. Therefore, there remains a significant need for improved design for bi-specific or multi-specific antibodies to provide for both desired efficacy and reduced adverse effects.

### SUMMARY OF THE INVENTION

In one aspect, the present invention provides an engineered antibody, comprising: (i) a first polypeptide comprises a first heavy chain variable domain (VH1) linked to a second light chain variable domain (VL2); (ii) a second polypeptide comprises a second heavy chain variable domain (VH2) linked to a first light chain variable domain (VL1); (iii) a third polypeptide comprises a third heavy chain variable domain (VH3); and (iv) a fourth polypeptide comprises a fourth light chain variable domain (VL3). In some embodiments, the VL1 and the VH1 associate to form a first domain capable of binding to a first target; the VL2 and the VH2 associate to form a second domain capable of binding to a second target; the VL3 and the VH3 associate to form a third domain capable of binding to a third target. In such embodiments, the second and the third polypeptide chain further comprises a first dimerization domain and a second dimerization domain, respectively, that are associated to form a dimer. In some embodiments, one of the first target and the second target is an immune stimulatory target; and the other two targets are different epitopes on a same tumor antigen that is capable of dimerization upon binding to a native ligand.

In some embodiments, the N-terminus of said VL1 is linked to C-terminus of said VH2, and N-terminus of said VL2 is covalently linked to C-terminus of said VH1. In some embodiments, the N-terminus of said VH1 is linked to C-terminus of said VL2, and N-terminus of said VH2 is linked to C-terminus of said VL1.

In some embodiments, the immune stimulatory target is selected from NKG2D, CD3 and CD16.

In some of these embodiments, the immune stimulatory target is NKG2D. In certain embodiments, the domain capable of binding to NKG2D comprises: (a) a VL comprising an LCDR1 comprising a sequence as shown in SEQ ID NO: 29, an LCDR2 comprising a sequence as shown in SEQ ID NO: 30, an LCDR3 comprising a sequence as shown in SEQ ID NO: 31, and a VH comprising an HCDR1 comprising a sequence as shown in SEQ ID NO: 25, an HCDR2 comprising a sequence as shown in SEQ ID NO: 26, and an HCDR3 comprising a sequence as shown in SEQ ID NO: 27; or (b) a VL comprising an LCDR1 comprising a sequence as shown in SEQ ID NO: 118, an LCDR2 comprising a sequence as shown in SEQ ID NO: 119, an LCDR3 comprising a sequence as shown in SEQ ID NO: 120, and a VH comprising an HCDR1 comprising a sequence as shown in SEQ ID NO: 114, an HCDR2 comprising a sequence as shown in SEQ ID NO: 115, and an HCDR3 comprising a sequence as shown in SEQ ID NO: 116. In certain embodiments, the VL1 comprises a sequence of SEQ ID NO: 32, and the VH1 comprises a sequence of SEQ ID NO: 28, or the VL comprises a sequence of SEQ ID NO: 121, and the VH comprises a sequence of SEQ ID NO: 117.

In some other of these embodiments, the immune stimulatory target is CD3. In certain embodiments, the domain capable of binding to CD3 comprises a VL comprising an LCDR1 comprising a sequence as shown in SEQ ID NO: 5, an LCDR2 comprising a sequence as shown in SEQ ID NO: 6, an LCDR3 comprising a sequence as shown in SEQ ID NO: 7, and a VH comprising an HCDR1 comprising a sequence as shown in SEQ ID NO: 1, an HCDR2 comprising a sequence as shown in SEQ ID NO: 2, and an HCDR3 comprising a sequence as shown in SEQ ID NO: 3. In certain embodiments, the VL1 comprises a sequence of SEQ ID NO: 8, and the VH1 comprises a sequence of SEQ ID NO: 4.

In some other of these embodiments, the immune stimulatory target is CD16. In certain embodiments, the domain capable of binding to CD16 comprises a VL comprising an LCDR1 comprising a sequence as shown in SEQ ID NO: 37, an LCDR2 comprising a sequence as shown in SEQ ID NO: 38, an LCDR3 comprising a sequence as shown in SEQ ID NO: 39, and a VH comprising an HCDR1 comprising a sequence as shown in SEQ ID NO: 33, an HCDR2 comprising a sequence as shown in SEQ ID NO: 34, and an HCDR3 comprising a sequence as shown in SEQ ID NO: 35. In certain embodiments, the VL1 comprises a sequence of SEQ ID NO: 40, and the VH1 comprises a sequence of SEQ ID NO: 36.

In some embodiments, the tumor antigen is HER2.

In some embodiments, one of domain capable of binding to HER2 is a first HER2 binding domain, which is derived from the antigen-binding domain of Trastuzumab, and the other domain capable of binding to HER2 is a second HER2 binding domain, which is derived from the antigen-binding domain of Pertuzumab. In some embodiments, the VL2 and the VH2 associate to form the first HER2 binding domain, and the VL3 and the VH3 associate to form the second HER2 binding domain. In some embodiments, the VL2 and the VH2 associate to form the second HER2 binding domain, and the VL3 and the VH3 associate to form the first HER2 binding domain. In some embodiments, the VL1 and the VH1 associate to form the first HER2 binding domain, and the VL3 and the VH3 associate to form the second HER2 binding domain. In some embodiments, the VL1 and the VH1 associate to form the second HER2 binding domain, and the VL3 and the VH3 associate to form the first HER2 binding domain.

In some embodiments, the first HER2 binding domain comprises an LCDR1 comprising a sequence as shown in SEQ ID NO: 13, an LCDR2 comprising a sequence as shown in SEQ ID NO: 14, an LCDR3 comprising a sequence as shown in SEQ ID NO: 15 or SEQ ID NO: 97, and the VH2 comprises an HCDR1 comprising a sequence as shown in SEQ ID NO: 9, an HCDR2 comprising a sequence as shown in SEQ ID NO: 10, and an HCDR3 comprising a sequence as shown in SEQ ID NO: 11.

In some embodiments, the second HER2 binding domain comprises an LCDR1 comprising a sequence as shown in SEQ ID NO: 21, an LCDR2 comprising a sequence as shown in SEQ ID NO: 22, an LCDR3 comprising a sequence as shown in SEQ ID NO: 23, and the VH3 comprises an HCDR1 comprising a sequence as shown in SEQ ID NO: 17, an HCDR2 comprising a sequence as shown in SEQ ID NO: 18, and an HCDR3 comprising a sequence as shown in SEQ ID NO: 19.

In some embodiments, the VL1 and the VH1 are associated with a first non-native disulfide bond.

In some embodiments, the first non-native disulfide bond is formed between two non-native cysteine residues in the VL1 and the VH1, respectively, optionally, the two non-native cysteine residues are Q100C in the VL1 and G44C in the VH1, wherein numbering is according to the Kabat index. In some of these embodiments, the VL2 and the VH2 are associated with either a native disulfide bond or a different non-native disulfide bond formed at positions distinct from those of the first non-native disulfide bond.

In some embodiments, the VL2 and the VH2 are associated with a second non-native disulfide bond.

In some embodiments, the second non-native disulfide bond is formed between two non-native cysteine residues in the VL2 and the VH2, respectively, optionally, the two non-native cysteine residues are Q100C in the VL2 and G44C in the VH2, wherein numbering is according to the Kabat index. In some of these embodiments, the VL1 and the VH1 are associated with either a native disulfide bond or a different non-native disulfide bond formed at positions distinct from those of the second non-native disulfide bond.

In some embodiments, the VL1 and the VH1 are further associated with electrostatic interactions between two oppositely charged residues.

In some embodiments, the VL2 and the VH2 are further associated with electrostatic interactions between two oppositely charged residues.

In some embodiments, the two oppositely charged residues are introduced to replace Q38 in the VL1 and Q39 in the VH1, respectively; or to replace Q38 in the VL2 and Q39 VH2, respectively, wherein numbering is according to the Kabat index.

In some embodiments, the two oppositely charged residues are introduced to replace Q40 in the VL1 and Q39 in the VH1, respectively; or to replace Q40 in the VL2 and Q39 VH2, respectively, wherein numbering is according to the Kabat index.

In some embodiments, the two oppositely charged residues are introduced to replace Q37 in the VL1 and Q39 in the VH1, respectively; or to replace Q37 in the VL2 and Q39 VH2, respectively, wherein numbering is according to the Kabat index.

In some embodiments, the two oppositely charged residues comprises a negatively charged amino acid residue selected from aspartic acid (D) or glutamic acid (E), and a positively charged amino acid residue selected from lysine (K), histidine (H) or arginine (R).

In some embodiments, the two oppositely charged residues comprise Q38D in the VL1 and Q39K in the VH1, respectively; or Q38D in the VL2 and Q39K in the VH2, respectively, wherein numbering is according to the Kabat index.

In some embodiments, the VH2 comprises a sequence as shown in SEQ ID NO: 90 and the VL2 comprises a sequence as shown in SEQ ID NO: 91. In such embodiments, the VH3 comprises a sequence as shown in SEQ ID NO: 20 and the VL3 comprises a sequence as shown in SEQ ID NO: 24; or the VH3 comprises a sequence as shown in SEQ ID NO: 106 and the VL3 comprises a sequence as shown in SEQ ID NO: 107.

In some embodiments, the VH2 comprises a sequence as shown in SEQ ID NO: 92 and a VL2 comprising a sequence as shown in SEQ ID NO: 93. In such embodiments, the VH3 comprises a sequence as shown in SEQ ID NO: 12 and a VL3 comprising a sequence as shown in SEQ ID NO: 16.

In some embodiments, the VH2 comprises a sequence as shown in SEQ ID NO: 90 and a VL2 comprising a sequence as shown in SEQ ID NO: 98. In such embodiments, the VH3 comprises a sequence as shown in SEQ ID NO: 12 and a VL3 comprising a sequence as shown in SEQ ID NO: 95.

In some embodiments, the VH2 comprises a sequence as shown in SEQ ID NO: 90 and a VL2 comprising a sequence as shown in SEQ ID NO: 91. In such embodiments, the VH3 comprises a sequence as shown in SEQ ID NO: 12 and a VL3 comprising a sequence as shown in SEQ ID NO: 16.

In some embodiments, the VL1 and the VH1 associate to form the first domain capable of binding to NKG2D, the VL2 and the VH2 associate to form the first HER2 binding domain, and the VL3 and the VH3 associate to form the second HER2 binding domain.

In some of these embodiments, (i) the VL1 comprises a sequence of SEQ ID NO: 32, and the VH1 comprises a sequence of SEQ ID NO: 28; (ii) the VL2 comprises a sequence of SEQ ID NO: 91, and the VH2 comprises a sequence of SEQ ID NO: 90; and (iii) the VL3 comprises a sequence of SEQ ID NO: 24, and the VH3 comprises a sequence of SEQ ID NO: 20.

In some embodiments, the VL1 and the VH1 associate to form the first domain capable of binding to CD3, the VL2 and the VH2 associate to form the second HER2 binding domain, and the VL3 and the VH3 associate to form the first HER2 binding domain.

In some of these embodiments, (i) the VL1 comprises a sequence of SEQ ID NO: 8, and the VH1 comprises a sequence of SEQ ID NO: 4; (ii) the VL2 comprises a sequence of SEQ ID NO: 93, and the VH2 comprises a sequence of SEQ ID NO: 92; and (iii) the VL3 comprises a sequence of SEQ ID NO: 16, and the VH3 comprises a sequence of SEQ ID NO: 12.

In some embodiments, the VL1 and the VH1 associate to form the first domain capable of binding to CD16, the VL2 and the VH2 associate to form the second HER2 binding domain, and the VL3 and the VH3 associate to form the first HER2 binding domain.

In some of certain embodiments, (i) the VL1 comprises a sequence of SEQ ID NO: 40, and the VH1 comprises a sequence of SEQ ID NO: 36; (ii) the VL2 comprises a sequence of SEQ ID NO: 93, and the VH2 comprises a sequence of SEQ ID NO: 92; and (iii) the VL3 comprises a sequence of SEQ ID NO: 16, and the VH3 comprises a sequence of SEQ ID NO: 12.

In some embodiments, the VL1 and the VH1 associate to form the first domain capable of binding to CD3, the VL2 and the VH2 associate to form a third HER2 binding domain, and the VL3 and the VH3 associate to form a fourth HER2 binding domain. In certain embodiments, the third HER binding domain is derived from the antigen-binding domain of Trastuzumab and further modified to have reduced affinity to HER2.

In some of these embodiments, (i) the VL1 comprises a sequence of SEQ ID NO: 100, and the VH1 comprises a sequence of SEQ ID NO: 99; (ii) the VL2 comprises a sequence of SEQ ID NO: 98, and the VH2 comprises a sequence of SEQ ID NO: 90; and (iii) the VL3 comprises a sequence of SEQ ID NO: 95, and the VH3 comprises a sequence of SEQ ID NO: 12.

In some embodiments, the VL1 is linked to the VH2 via a first peptide linker, and wherein the VL2 is linked to the VH1 via a second peptide linker.

In certain embodiments, the first peptide linker and the second peptide linker each independently comprises 5 to 9 amino acids.

In certain embodiments, the first peptide linker and the second peptide linker each comprises a sequence as shown in SEQ ID NO: 94.

In some embodiments, the first dimerization domain and the second dimerization domain comprise CH3 domain of IgG, optionally further comprising one or more mutations that facilitate heterodimerization.

In some embodiments, the first dimerization domain comprises a first mutation, and the second dimerization domain comprises a second mutation.

In certain embodiments, a) the first mutation comprises T389W and/or S375C, and the second mutation comprises Y438V, T389S, L391A, and/or Y370C; b) the first mutation comprises D427K and/or D377K, and the second mutation comprises K420D, and/or K440D; c) the first mutation comprises D377K, E378K, and/or D427K, and the second mutation comprises K393E, K440D, and/or K470E; d) the first mutation comprises S387H, and/or F436A, and the second mutation comprises Y370T, and/or T422F; e) the first mutation comprises S387H, and/or T422F, and the second mutation comprises Y422T, and/or F436A; f) the first mutation comprises K393D, and/or K440D, and the second mutation comprises E378K, and/or D427K; or g) the first mutation comprises L372D, and/or L391E, and the second mutation comprises L372K, or T389K, wherein numbering is according to the Kabat index.

In certain embodiments, the first mutation comprises T389W and the second domain comprises T389S, L391A and Y438V, wherein numbering is according to the Kabat index.

In some embodiments, the first dimerization domain and/or the second dimerization domain further comprise CH2 domain and/or hinge region of IgG.

In some embodiments, the hinge region comprises a sequence as shown in SEQ ID NO: 43, 101 or 102.

In some embodiments, the third polypeptide further comprises CH1 region. In some of these embodiments, the CH1 region comprises a sequence as shown in SEQ ID NO: 41.

In some embodiments, the fourth polypeptide further comprises CL region. In some of these embodiments, the CL region comprises a sequence as shown in SEQ ID NO: 42.

In some embodiments, one of the first dimerization domain and the second dimerization domain comprises a sequence as shown in SEQ ID NO: 44 or 103, and the other comprises a sequence as shown in SEQ ID NO: 45 or 104.

In some embodiments, the first dimerization domain and the second dimerization domain retain the ADCC activity, wherein one of the first dimerization domain and the second dimerization domain comprises a sequence as shown in SEQ ID NO: 103, and the other comprises a sequence as shown in SEQ ID NO: 104.

In some embodiments, the first dimerization domain and the second dimerization domain do not retain the ADCC activity, wherein one of the first dimerization domain and the second dimerization domain comprises a sequence as shown in SEQ ID NO: 44, and the other comprises a sequence as shown in SEQ ID NO: 45.

In some embodiments, the first polypeptide comprises a sequence as shown in SEQ ID NO: 58, the second polypeptide comprises a sequence as shown in SEQ ID NO: 59, the third polypeptide comprises a sequence as shown in SEQ ID NO: 20, and the fourth polypeptide comprises a sequence as shown in SEQ ID NO: 24.

In some embodiments, the first polypeptide comprises a sequence as shown in SEQ ID NO: 62, the second polypeptide comprises a sequence as shown in SEQ ID NO: 63, the third polypeptide comprises a sequence as shown in SEQ ID NO: 12, and the fourth polypeptide comprises a sequence as shown in SEQ ID NO: 16.

In some embodiments, the first polypeptide comprises a sequence as shown in SEQ ID NO: 74, the second polypeptide comprises a sequence as shown in SEQ ID NO: 75, the third polypeptide comprises a sequence as shown in SEQ ID NO: 12, and the fourth polypeptide comprises a sequence as shown in SEQ ID NO: 16.

In some embodiments, the engineered antibody described herein is linked to one or more conjugate moieties.

In some embodiments, the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs

In one aspect, the present disclosure provides an isolated polynucleotide encoding the engineered antibody described herein.

In another aspect, the present disclosure provides a vector comprising the isolated polynucleotide described herein.

In yet another aspect, the present disclosure provides a host cell comprising the vector described herein.

In yet another aspect, the present disclosure provides a pharmaceutical composition comprising: (i) the engineered antibody described herein, or the polynucleotide encoding the engineered antibody described herein; and (ii) one or more pharmaceutically acceptable carriers, diluent, buffer or excipient.

In some embodiments, the pharmaceutical composition described herein further comprises an additional therapeutic agent.

In some embodiments, the additional therapeutic agent is an agent for treating a HER2-related disease or disorder.

In some embodiments, the HER2-related disease or disorder is a cancer, which is selected from the group consisting of: breast cancer, prostate cancer, lung cancer, bladder cancer, ovarian cancer, gastric cancer, colorectal cancer, esophageal cancer, head & neck cancer, cervical cancer, pancreatic cancer, testis cancer, urothelial cancer, endometrial cancer, malignant melanoma, and a soft-tissue cancer (e.g., synovial sarcoma).

In yet another aspect, the present disclosure provides a method of expressing the engineered antibody described herein, comprising culturing the host cell described herein under the condition at which the vector described herein is expressed.

In yet another aspect, the present disclosure provides a method of treating, preventing or alleviating a disease or disorder in a subject, comprising administering to the subject a therapeutically effective amount of the engineered antibody described herein.

In yet another aspect, the present disclosure provides a method of treating, preventing or alleviating a disease or disorder in a subject, comprising administering to the subject a therapeutically effective amount of the engineered antibody described herein, or the polynucleotide encoding the engineered antibody described herein, and/or the pharmaceutical composition described herein.

In some embodiments, the disease or disorder is a HER2-related disease or disorder.

In some embodiments, the subject is human.

In some embodiments, the administration is via oral, nasal, intravenous, subcutaneous, sublingual, or intramuscular administration.

In yet another aspect, the present disclosure provides use of the engineered antibody described herein, the pharmaceutical composition described herein, and/or the polynucleotide encoding the engineered antibody described herein in the manufacture of a medicament for treating, preventing or alleviating a disease or disorder.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the schematic diagram of DICAD structure.

FIG. 2 depicts the schematic diagram of TRIAD structure.

FIG. 3 shows the effect of Herceptin, Perjeta, 6A1 and 6A2 on BT-474 cells proliferation (dose-effect curves).

FIG. 4A shows the results of killing of SK-BR-3 cells by 6A8, 6A12, 6A14, 6A15 and Herceptin.

FIG. 4B shows the results of killing of SK-BR-3 cells by 6A15, Herceptin, 6A17, 6A14, 6A8, 6A21, 6A19 and 6B1.

FIG. 4C shows the results of killing of SK-BR-3 cells by 6A14, 6A17, 6A18 and Trastuzumab.

FIG. 4D shows the results of killing of SK-BR-3 cells by 6B1, 6A23, 6A25 and 6A26.

FIG. 4E shows the results of killing of SK-BR-3 cells by 6A17, 6A18 and 6A19.

FIG. 4F shows the results of killing of SK-BR-3 cells by FT-1, 6B1 and 6A23.

FIG. 5A shows the results of killing of MCF-7 cells by 6B1, 6A23, 6A25 and 6A26.

FIG. 5B shows the results of killing of MCF-7 cells by 6A17, 6A18, 6A19, FT-1, 6B1 and 6A23.

FIG. 6 shows the effects of 6B1, 6A25, 6A19 and 6A26 on the growth of subcutaneous transplanted tumors of human breast cancer KPL-4 cells.

FIG. 7 shows the effects of 6B1, 6A19, 6A25 and 6A26 on the growth of subcutaneous transplanted tumors of human breast cancer BT474 cells.

FIG. 8 shows the effects of 6B1, 6A19, 6A25 and 6A26 on the growth of subcutaneous transplanted tumors of human colon cancer HT55 cells.

FIG. 9 shows the effects of control, 6B1, 6A19, 6A25 and 6A26 of on the growth of subcutaneous transplanted prostate cancer cell line PC-3.

FIG. 10 shows the exemplary constant region sequences of the engineered antibodies.

FIG. 11 shows the sequences of the control antibodies 6A1 and 6A2.

FIG. 12 shows the sequences of the control antibody 6B1.

FIG. 13 shows the VH and VL sequences targeting CD3, HER2 (namely Tratuzumab and Pertuzumab), CD16 and NKG2D respectively, with mutations introducing non-native disulfide bonds and/or charged amino acids.

### DETAILED DESCRIPTION OF THE INVENTION

Before the detailed description of the inventions is provided, the following are noted and defined.

All the description provided herein is merely intended to illustrate various embodiments of the inventions provided in the present disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein.

All references cited in the present disclosure, including patent applications, issued patents, published articles or other publications, are incorporated by reference in their entirety, which are for the purpose of providing methodologies that might be used in connection with the description provided herein. With respect to any term that is presented in one or more publications that is similar to, or identical with, a term that has been expressly defined in this disclosure, the definition of the term as expressly provided in this present disclosure will control in all respects.

All technical and scientific terms used, unless expressly defined otherwise, in this present disclosure, are generally deemed to have the same meaning as commonly understood to one of ordinary skill in the art to which this disclosure belongs.

As used herein, i.e., throughout the whole disclosure, the articles “a,” “an,” and “the” are to be construed to mean “one or more” or “at least one” unless specified otherwise. By way of example, “a polypeptide complex” means one polypeptide complex or more than one polypeptide complex.

As used herein, the terms “about,” “approximately,” “around” or alike, refer to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “comprise,” “comprises,” “comprising,” “include,” “includes,” “including,” “contain,” “contains,” “containing”, “have,” “has,” “having” and the like, are synonymous and are used inclusively, in an open-ended fashion, and do not exclude additional elements, features, steps, acts, operations, and so forth.

As used herein, the term “or” is used in its inclusive sense (and not in its exclusive sense) so that when used, for example, to connect a list of elements, the term “or” means one, some, or all of the elements in the list.

As used herein, the phrase “at least one” means one or more, i.e. 1, 2, 3, 4, 5, 6, 7, 8, 9, 10, or more. A phrase referring to “at least one of” a list of items is construed to refer to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiment requires at least one of X, at least one of Y, and at least one of Z to each be present.

As used herein, the references “one embodiment,” “an embodiment,” “a particular embodiment,” “a related embodiment,” “a certain embodiment,” “an additional embodiment,” “some embodiments,” “certain embodiments,” or “a further embodiment” or combinations thereof, are to be understood to mean that a particular feature, structure or characteristic described in connection with this particular embodiment is included in at least one embodiment of the present disclosure. Thus, the presences or appearances of the foregoing phrases in various places throughout this disclosure are not necessarily all referring to the same embodiment. Furthermore, the particular features, structures, or characteristics may be combined in any suitable manner in one or more embodiments.

Conditional language used herein, such as, among others, “can,” “could,” “might,” “may,” “e.g.,” and the like, unless stated expressly otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

### I. Definitions and Abbreviations

In this section, definitions for some general terms are provided. Definition for other terms may be found in other sections of the disclosure that follow.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein, the term “amino acid” refers to a building block of a protein, a peptide, a polypeptide or an amino acid polymer, and the term “amino acid” further refers to a naturally occurring or synthetic amino acid, as well as any amino acid analog and amino acid mimetics that functions in a manner similar to the naturally occurring amino acid. Naturally occurring amino acids are those encoded by the genetic codes, as well as those amino acids that are later modified, e.g., hydroxyproline, gamma-carboxyglutamate, and O-phosphoserine. As used within this application, naturally occurring amino acids include the group of naturally occurring carboxy alpha-amino acids comprising alanine (three letter code: Ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

As used herein, the term “domain” refers to a globular structure formed by one or more regions of one or more polypeptide chains comprising peptide loops (e.g., comprising 3 to 4 peptide loops) that are stabilized, for example, by β-pleated sheet and/or intrachain disulfide bond(s). Examples may include a Fab domain (see below for more details). It is noted that in the present disclosure, the two terms “domain” and “region” may be used interchangeably.

As used herein, the term “nucleic acid,” “nucleic acid molecule,” “nucleotide,” “polynucleotide” or alike, is construed to refer to a nucleotide polymer of any length, and can include both DNA and RNA, and can be single-stranded or double-stranded.

The term “antibody” as used herein encompasses any immunoglobulin, monoclonal antibody, polyclonal antibody, chimeric antibody, humanized antibody, multispecific antibody, bispecific antibody, bivalent antibody or multivalent antibody that binds to a specific antigen. In the following, a description of an antibody, as well as terms relevant thereto, is provided in more detail.

In mammals such as human, depending on the different types of heavy chains present in the immunoglobulins, there are five different classes/isotypes (i.e. IgA, IgD, IgE, IgG, and IgM, corresponding to the five Ig heavy chain types α, δ, ε, γ, and μ, respectively) of antibodies, which typically have different molecular and biological properties, functional locations, physiological functionalities, and pathological implications in diseases. Certain antibody classes may further include subclasses. For example, in human, IgA may include IgAQ1 and IgA2 subclasses, and IgG may include four subclasses denoted as IgG1, IgG2, IgG3, and IgG4, respectively. With an immunoglobulin monomer as its basic functional unit, a mammalian antibody may exist as a monomer (e.g. IgD, IgE, and IgG), a dimer (IgA), a tetramer (IgM), or a pentamer (IgM). In mammals, two types of light chain exist, including kappa (κ) chain and lambda (λ) chain.

Within the basic immunoglobulin unit, a native or naturally occurring antibody such as IgG generally includes two identical heavy (H) chains and two identical light (L) chains. Each light chain is linked to a heavy chain by one covalent disulfide bond or linkage formed between a pair of cysteine residues present respectively in the each light chain and the heavy chain, and the two heavy chains are further linked to one another through several disulfide bonds formed between cysteine residues in each heavy chain. The tetramer thus formed substantially takes a Y-like shape for the antibody, with the end of each fork arm containing an identical antigen-binding site (i.e. paratope) that interacts specifically with a corresponding epitope of the antigen.

More specifically, in a native antibody, in a N-terminal-to-C-terminal direction, each heavy chain includes a variable region (VH, or HCVR), followed by three or four constant regions (“CHs”, with IgA, IgD, IgG containing three CH regions CH1, CH2 and CH3; and IgE and IgM containing four CH regions CH1, CH2, CH3 and CH4), and each light chain includes a variable region (VL, or LCVR) and a constant region (CL). In the Y-shaped antibody, the variable region of each light chain (i.e. the VL region) aligns or associates with the variable region of its pairing heavy chain (i.e. the VH region) to together form an antigen-binding site for the antibody.

The term “variable region” or “VR” as used herein means the region in heavy chain or light chain of an antibody that are responsible for antigen binding. In a native antibody, the heavy chain variable region (VH or HCVR) contains three highly variable loops called “complementarity determining regions” (CDRs), i.e., heavy (H) chain CDRs including HCDR1, HCDR2, HCDR3, and the light chain variable region (VL or LCVR) contains three light (L) chain CDRs including LCDR1, LCDR2, and LCDR3. CDR boundaries for antibodies may be defined or identified by the conventions of Kabat, Chothia, or Al-Lazikani (Al-Lazikani, B., Chothia, C., Lesk, A. M., J. Mol. Biol., 273 (4), 927 (1997); Chothia, C. et al., J Mol. Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., National Institutes of Health, Bethesda, Md. (1991)). The three CDRs are interposed between flanking stretches known as “framework regions” (FRs), which are more highly conserved than the CDRs and form a scaffold to support the hypervariable loops. In a native antibody, each VH and VL comprises four FRs, and the CDRs and FRs are arranged from amino terminus to carboxyl terminus in the order: FR1, CDR1, FR2, CDR2, FR3, CDR3, FR4. However, it should be understood that the term “variable region” as used herein does not necessarily need to include all of the three CDRs or all of the four FRs, and should be construed to encompass any variant or derivative of a native variable region from a native antibody, as long as such variant or derivative retains antigen-binding activity.

The term “constant region” or “constant moiety” as used herein means the region in heavy chain or light chain of an antibody that are not directly involved in antigen binding. It should be understood that the term “constant region” or “constant moiety” as used herein does not necessarily need to include the full length of a native constant region of a native antibody, and should be construed to encompass any variant or derivative of such a native constant region or constant moiety, as long as such variant or derivative retains the ability to, for example, support stability of the antigen-binding domain, or retains the intended biological function such as effector functions such as secretion, transplacental mobility, Fc receptor binding, complement binding, and the like.

The term “CL region” refers to the constant region of an immunoglobulin light chain that is adjacent to the VL region. CL region may span from about Kabat position 108 to about Kabat position 216 in an immunoglobulin light chain. In a native antibody, the constant region of each light chain (i.e. the CL region) associates with the first constant region of a pairing heavy chain (i.e. the CH1 region).

The term “CH1 region” as used herein encompasses the first (most amino terminal) constant region of an immunoglobulin heavy chain that extends from, about Kabat position 114 to at least about Kabat position 233 (e.g. can be extended to Kabat position 234, and so on). The CH1 region is adjacent to the VH region, and is amino terminal to the hinge region of an immunoglobulin heavy chain molecule.

The term “hinge region” in terms of an antibody includes the portion of a heavy chain molecule that joins the CH1 region to the CH2 region. The length of hinge region may vary depending on the defined boundaries of the CH1 region and of the CH2 region. The hinge region is normally flexible, thus allowing the two N-terminus antigen binding regions to move independently.

The term “CH2 region” as used herein refers to the portion of a heavy chain immunoglobulin molecule that extends, e.g., from about Kabat position 244 to Kabat position 360.

The term “CH3 region” as used herein refers to the portion of a heavy chain immunoglobulin molecule that extends approximately 110 residues from N-terminus of the CH2 domain, e.g., from about Kabat position 361 to Kabat position 476, 477 or 478. The CH3 domain typically forms the C-terminal portion of the antibody like IgG, IgA, and IgD. In some immunoglobulins like IgE and IgM, however, additional domains may extend from CH3 domain to form the C-terminal portion of the molecule (e.g. the CH4 domain in the u chain of IgM and the & chain of IgE).

“Fc” as used herein refers to a portion derived from an antibody, for example, IgG, mainly composed of the second (CH2) and third (CH3) constant regions of a first heavy chain bound to the CH2 and CH3 of a second heavy chain via one or more covalent bonds which are non-peptide bonds, for example, via disulfide bonding. The Fc portion of the antibody is responsible for various effector functions such as antibody-dependent cellular cytotoxicity (ADCC) and complement-dependent cytotoxicity (CDC), etc., but does not function in antigen binding.

An “antigen” or “Ag” as used herein refers to a compound, composition, peptide, polypeptide, protein or substance (e.g., polypeptide, carbohydrate, nucleic acid, lipid, or other naturally occurring or synthetic compound) that can be specifically recognized and bound by a component of the immune system, e.g., an antibody. As used herein, the term “antigen” encompasses antigenic epitopes, e.g., fragments of an antigen which are antigenic epitopes. The term “antigen” and “target” are used interchangeably in the present disclosure.

An “epitope” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). An epitope typically includes at least 3, and more usually, at least 5, about 7, or about 8-10 amino acids in a unique spatial conformation.

As used herein, the term “antibody fragment” refers to a portion of, or derived from, a full-length antibody. An antibody fragment can be an antigen-binding fragment or variable region thereof.

As used herein, the term “antigen-binding domain” or alike, is referred to as antibody fragment, or a domain derived from a portion of an antibody, that comprises one or more CDRs or otherwise binds to an antigen but does not comprise an intact native antibody structure. Examples of antigen-binding fragments may include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody, etc. An antigen-binding fragment is capable of binding to the same antigen to which the parent full-length antibody binds. An antigen-binding fragment may comprise one or more CDRs from a particular human antibody grafted to a framework region from one or more different human antibodies. For more and detailed formats of antigen-binding moiety are described in Spiess et al., 2015 (Supra), and Brinkman et al., mAbs, 9 (2), pp. 182-212 (2017), which are incorporated herein by entirety reference.

As used herein, the term “Her2 binding domain” means an antigen-binding domain where the antigen is Her2.

“Fv” with regard to an antibody refers to the smallest fragment of the antibody to bear the complete antigen-binding site. An Fv fragment consists of the variable domain of a single light chain bound to the variable domain of a single heavy chain. A number of Fv designs have been provided, including dsFvs, in which the association between the two domains is enhanced by an introduced disulfide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerized to form diabodies and triabodies (Maynard et al., Annu Rev Biomed Eng 2 339-376 (2000)).

“Fab” as used herein refers to a single antigen-binding domain derived from an antibody, where the domain has a single heavy chain fragment associated with a single light chain fragment via one or more covalent bonds which are non-peptide bonds. In some embodiments, the single heavy chain fragment in the Fab domain comprises an HCVR and a CH1 region. In some embodiments, the single light chain fragment in the Fab domain comprises an LCVR and a CL domain. In some embodiments, the CH1 region associates with the HCVR by a covalent bond such as a disulfide bond. In a native antibody, the Fab domain corresponds substantially to one arm of the antibody, typically retains the ability to recognize and bind to its corresponding antigen.

“Fab” refers to a Fab fragment that includes a portion of the hinge region. “F(ab′)₂” refers to a dimer of Fab′.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85: 5879 (1988)).

“Single-chain Fv-Fc antibody” or “scFv-Fc” refers to an engineered antibody consisting of an scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231 (1-2): 25-38 (1999); Muyldermans S., J Biotechnol. June; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen VK. et al. Immunogenetics. April; 54 (1): 39-47 (2002); Nguyen VK. et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. November; 21 (13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate, and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv) 2” or “(dsFv-dsFv′)” comprises three peptide chains: two V_{H} moieties linked by a peptide linker (e.g., a long flexible linker) and bound to two V_{L} moieties, respectively, via disulfide bridges. In some embodiments, dsFv-dsFv′ is bispecific in which each disulfide paired heavy and light chain has a different antigen specificity.

As used herein, the term “multispecific antibody” refers to an artificial or engineered antibody that can simultaneously bind to at least two different epitopes. A bispecific antibody is substantially a type of a multispecific antibody. In addition, other multispecific antibodies may include trispecific antibodies, which have three different antigen-binding specificities, tetraspecific antibodies, which have four different antigen-binding specificities, and so on.

As used herein, the term “bispecific antibody” refers to an antibody that comprises two physically separable antigen-binding domains which differ from one another in their antigen specificity. Usually, a bispecific antibody is an artificial antibody which has fragments derived from two different monoclonal antibodies and is capable of binding to two different epitopes. The two epitopes may present on the same antigen, or they may present on two different antigens. It is in contrast to a naturally occurring antibody which has two physically separable antigen-binding moieties that are structurally identical and thus have the same antigen specificity.

The term “affinity” as used herein refers to the strength of non-covalent interaction between an immunoglobulin molecule (i.e. antibody) or fragment thereof and an antigen. Affinity of an antibody to an antigen can be measured by the equilibrium dissociation constant K_{D} using methods well known in the art (see, generally, Davies et al. Ann. Rev. Biochem. 1990, 59:439-15 473).

The term “variant” refer to without limitation any antibody which has a structure or sequence derived from the antibodies of the present disclosure and whose structure/sequence is sufficiently similar to those disclosed herein and based upon that similarity, would be expected, by one skilled in the art, to exhibit the same or similar activities and utilities as the claimed and/or referenced antibody, thereby also interchangeably referred to “functional equivalent”. Modifications to obtain “variant” includes, for example, by addition, deletion and/or substitution of one or more of the amino acid residues. The functional equivalent or fragment of the functional equivalent may have one or more conservative amino acid substitutions. The term “conservative amino acid substitution” refers to substitution of an amino acid to another amino acid that has similar properties to the original amino acid. The groups of conservative amino acids are known in the art.

Conservative substitutions may be introduced in any position of a preferred predetermined peptide or fragment thereof. It may however also be desirable to introduce nonconservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

Throughout the disclosure, the numbers indicating the positions of amino acid residues in a constant region of an antibody, such as those in the heavy chain constant region 1 (CH1) and the light chain constant region (CL) in a constant moiety, are based on the Kabat index, as described in Kabat et al., Sequences of Proteins of Immunological Interest, 5th Ed. Public Health Service, National Institutes of Health, Bethesda, Md. (1991). As indicated, some positions use IMGT numbering or EU numbering, as described in Edelman, G. M. et al., Proc. Natl. Acad. USA, 63, 78-85 (1969); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015). These numberings are also available from the IMGT scientific chart, accessible from the website of international ImMunoGeneTics information system.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or an antigen-binding fragment thereof, at least one promoter (e.g., SV40, CMV, EF-1a) operably linked to the nucleic acid sequence, and at least one selection marker.

The phrase “host cell” as used herein refers to a cell into which an exogenous polynucleotide and/or a vector can be or has been introduced.

“HER2” (from human epidermal growth factor receptor 2) as used herein is a protein that in human encoded by the ErbB2 gene. ErbB is abbreviated from erythroblastic oncogene B, and it belongs to the epidermal growth factor receptor family and consists of an extracellular domain, a transmembrane domain, and a cytoplasmic tyrosine kinase domain. In humans, the ErbB family includes four members: ErbB1 (Her1), ErbB2 (Her2), ErbB3 (Her3), ErbB4 (Her4), among which HER2 is receptor tyrosine-protein kinase erbB-2, also known as HER2/neu, CD340 (cluster of differentiation 340), proto-oncogene Neu, Erbb2 (rodent), or ERBB2 (human).

### II. Engineered Antibodies

The present invention provides novel engineered antibodies. The term “engineered” with respect to an antibody refers to artificially constructed macromolecule incorporating an antibody or antigen-binding fragment thereof. An engineered antibody can be in a distinct format or construct from a conventional antibody, and/or can comprise mutations of amino acids in original antibody sequences.

The present invention provides novel engineered antibodies having three antigen-binding domains capable of binding to a first target and two epitopes on a second target, respectively. In some embodiments, the second target is an antigen that is capable of dimerization. The second target can be an antigen that is capable of dimerization or oligomerization upon binding to a ligand. The engineered antibodies of the present disclosure are designed to be capable of simultaneously binding to the two epitopes on the second target, such that it has increased affinity to the second target but does not induce dimerization or oligomerization of the second target. This design is particularly useful because it increases binding affinity to the second target without leading to unwanted activation of the second target as a result of its dimerization or oligomerization.

In certain embodiments, the engineered antibodies provided herein are based on the TRIAD format, which is disclosed in, for example PCT application WO2019/120245, incorporated herein to its entirety.

TRIAD is a platform for construction of antibodies with molecular weight of about 153 kDa and that were able to simultaneously recognize three antigens. In general, the antibodies provided in the TRIAD platform resemble a classic antibody. They have the following advantages: (1) retaining the properties of bivalent bi-specific antibodies: avidity, affinity, potency etc.; (a) having high stability and less aggregation; (3) easy for expression and purification, in comparison to other bi-specific or multi-specific antibodies; (4) having a structure that similar to native IgG, and thus have decreased immunogenicity; and (5) proved to have high potency both in vitro and in vivo, and have a long half-life compared to most other bi-specific or multi-specific antibodies.

The engineered antibodies provided herein based on the TRIAD format has a structure similar to native IgG. It is composed of four polypeptide chains, forming a Y-shaped structure with two arms and one stem. However, the engineered antibody provided herein is asymmetric, with one arm having two antigen-binding sites and the other arm having a third antigen-binding site.

To increase binding affinity to a given antigen, the two antigen-binding sites on the same arm can be designed to target the same antigen, while the third antigen-binding site on the other arm is designed to target the other antigen. However, it is surprisingly found by the inventors that, such a design could be detrimental when the antigen targeted by the two antigen-binding sites on the same arm is capable of dimerization (or oligomerization). This is because simultaneous binding of two antigen-binding sites on the same arm to the antigen (such as HER2) could lead to unwanted dimerization and activation of such antigen, which is opposite to the intended inhibition of the antigen.

It is further surprisingly found by the inventors that, when the two antigen-binding sites targeting the same antigen are designed to locate in different arms, the unwanted dimerization and activation of such an antigen could be significantly reduced, and at the same time, the binding affinity to such an antigen could be increased.

Accordingly, in some embodiments, the engineered antibody comprises: (i) a first polypeptide comprising a first heavy chain variable domain (VH1) linked to a second light chain variable domain (VL2); (ii) a second polypeptide comprising a second heavy chain variable domain (VH2) linked to a first light chain variable domain (VL1); (iii) a third polypeptide comprises a third heavy chain variable domain (VH3); and (iv) a fourth polypeptide comprises a fourth light chain variable domain (VL3); wherein, the VL1 and the VH1 associate to form a first domain capable of binding to a first target; the VL2 and the VH2 associate to form a second target; the VL3 and the VH3 associate to form a third target; wherein one of the first target and the second target is an immune stimulatory target; and the other two targets are different epitopes on a same tumor antigen that is capable of dimerization upon binding to a native ligand.

In some embodiments, the first target is an immune target, while the second target and the third target are different epitopes on a same tumor antigen that is capable of dimerization upon binding to a native ligand.

In some other embodiments, the second target is an immune target, while the first target and the third target are different epitopes on a same tumor antigen that is capable of dimerization upon binding to a native ligand.

In certain embodiments, the engineered antibody provided herein comprises the second domain and the third domain, both targeting the second target. The second domain comprises VL2 in the first polypeptide and VH2 in the second polypeptide, while the third domain comprises VH3 in the third polypeptide and VL3 in the fourth polypeptide. In other words, the second domain and the third domain, both targeting the second target, are located at distinct arms of the engineered antibody.

In some embodiments, the first epitope and the second epitope of the second target in the engineered antibody are different. In some embodiments, the first epitope and the second epitope of the second target in the engineered antibody are substantially the same or identical.

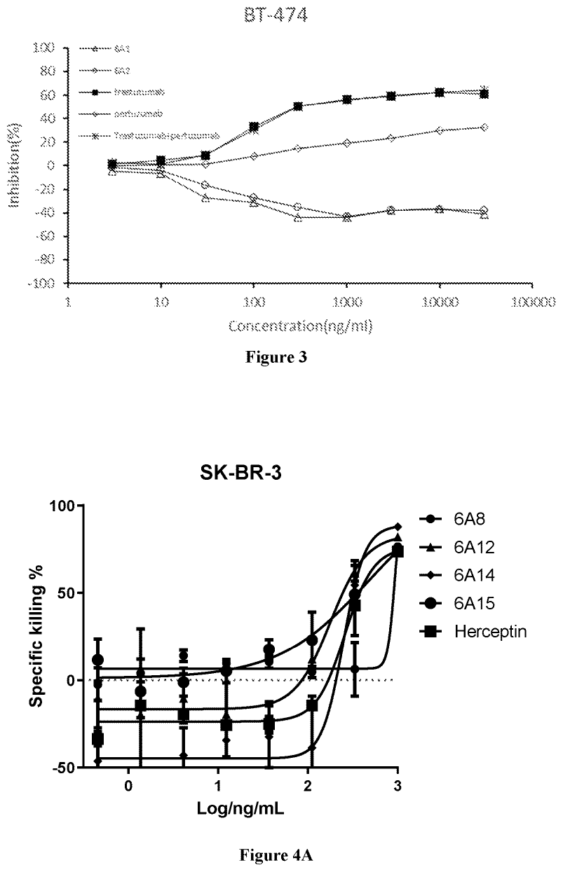

In some embodiments, the C-terminus of said VL1 is linked to N-terminus of said VH2, and C-terminus of said VL2 is covalently linked to N-terminus of said VH1. A schematic drawing the engineering antibody is provided in FIG. 2. In some embodiments, the C-terminus of said VH1 is linked to N-terminus of said VL2, and C-terminus of said VH2 is linked to N-terminus of said VL1.

In some embodiments, the VH2 and the VL1 and/or the VH1 and the VL2 are covalently linked either directly or indirectly, for example, via a linker, for example a peptide linker. The term “peptide linker” as used herein can be any suitable polypeptide capable of bonding two entities to thereby form one molecule, or maintaining association of the two entities in sufficiently close proximity, yet without substantially interference to the respective biological activities of the two entities. The linker can be made up of amino acid residues linked together by peptide bonds, yet may optionally further comprise one or more non-natural amino acids. Any suitable polypeptide can be used as a linker. In some embodiments, the polypeptide linker can be made up of a majority of amino acids that are sterically unhindered, such as glycine and alanine. In some embodiments, linkers are polyglycines, polyalanines, combinations of glycine and alanine (such as poly (Gly-Ala)), or combinations of glycine and serine (such as poly (Gly-Ser)). In some embodiments, the peptide linker comprises an amino acid sequence of RTVAA (SEQ ID NO: 94).

### A. Targets of the Engineered Antibodies

In certain embodiments, the second target is capable of dimerization or aggregation or crosslinking upon binding to its ligand. Exemplary antigens include, without limitation, receptor protein tyrosine kinases (RPTKs), class II cytokine receptor, and the like. In some embodiments, the RPTKs include EGF receptor family (also known as ErbB receptor family, including EGFR, ERBB2, ERBB3, ERBB4); Insulin receptor family (including INSR and IGFR); Platelet-derived growth factor receptors (PDGF receptor family, including PDGFRα, PDGFRβ, M-CSFR, KIT, FLT3L); VEGF receptor family (including VEGFR1, VEGFR2, VEGFR3); Fibroblast growth factor receptor family (FGF receptor family, including FGFR1, FGFR2, FGFR3, FGFR4); CCK receptor family (including CCK4); nerve growth factor receptor family (NGF receptor family, including TRKA, TRKB, TRKC); HGF receptor family (including MET and RON); Eph receptor family (including EPHA1 to 6, EPHB1 to 6); AXL receptor family (including AXL, MER, TYRO3); TIE receptor family (including TIE, TEK); RYK receptor family (including RYK); DDR receptor family (including DDR1, DDR2); RET receptor family (including RET); ROS receptor family (including ROS); LTK receptor family (including LTK, ALK); ROR receptor family (including ROR1, ROR2); MuSK receptor family (including MUSK); LMR receptor (including AATYK1, AATYK2, AATYK3); and others, such as RTK106. In some embodiments, the class II cytokine receptors include receptor of IFNα, IFNβ, IFNγ, IL-10 etc.

In most cases, the dimerization of this kind of antigens on the cell surface may lead to activation of a series of biochemical reactions in cells, and to cause comprehensive cell response. Taking HER2 for example, its dimerization may lead to cell proliferation, especially cancer cell proliferation.

In some embodiments, the second target recognized by the engineered antibody is a tumor antigen that is capable of dimerization upon binding to a ligand.

In some embodiments, the tumor antigen is HER2. HER2 belongs to the ErbB receptor family, consists of four plasma membrane-bound receptor tyrosine kinases. HER2 is known to be able to homo-dimerize and hetero-dimerize with HER1, HER3 or HER4. Dimerization of HER2 results in the autophosphorylation of tyrosine residues within the cytoplasmic domain of the receptors and initiates a variety of signaling pathways, including the PI3K/Akt and MAPK pathways in cells, thus regulating the proliferation, differentiation, migration and apoptosis of tumor cells. HER2 proteins have been shown to form clusters in cell membranes that may play a role in tumorigenesis.

Antibodies targeting HER2 is believed to be an effective strategy to fight the diseases related to HER2. Trastuzumab, sold under the brand name Herceptin among others, is a monoclonal antibody used to treat breast cancer and stomach cancer, in particular, HER2 positive cancers. Pertuzumab, sold under the brand name Perjeta, is another monoclonal antibody, which can be used in combination with trastuzumab and docetaxel for the treatment of metastatic HER2-positive breast cancer.

However, the inventors have surprisingly found that, when a TRIAD format antibody having two HER2 binding domains located at the same arm of the antibody, the TRIAD format antibody did not show HER2 blocking effect, but unexpectedly demonstrated a significant HER2 agonistic effect. For example, 6A1 and 6A2 recorded in the Example 2, constructed using the TRIAD model wherein each arm of the antibodies comprises two HER2 binding sites (Trastuzumab and Pertuzumab, which bind HER2 at ECD2 and ECD4, respectively), are not effective. This surprising finding indicates that when the two antigen-binding domains are too close together in the same arm of the antibodies, such as those constructed through the TRIAD platform, it will bring HER2 monomers closer to each other and enhances HER2 dimer formation, thereby promoting HER2-dependent tumor growth.

In contrast, in the engineered antibodies provided herein where the second domain and the third domain both targeting HER2 are located at different arms of the antibody, dimerization of HER2 is significantly reduced than otherwise located at the same arm, whereas the binding affinity to HER2 is at least maintained or even improved relative to a HER2 monovalent Ig-like bispecific antibody.

In some embodiments, in the engineered antibody, one of the second domain and the third domain is a first HER2 binding domain, which is derived from the antigen-binding domain of Trastuzumab, and the other is a second HER2 binding domain, which is derived from the antigen-binding domain of Pertuzumab. In other words, when the second domain is the first HER2 binding domain, the third domain would be the second HER2 binding domain; or when the third domain is the first HER2 binding domain, the second domain would be the second HER2 binding domain.

In some embodiments, in the engineered antibody, the VL2 and the VH2 associate to form the first HER2 binding domain, and the VL3 and the VH3 associate to form the second HER2 binding domain. In other words, the second domain is the first HER2 binding domain and the third domain is the second HER binding domain.

In some embodiments, in the engineered antibody, the VL2 and the VH2 associate to form the second HER2 binding domain, and the VL3 and the VH3 associate to form the first HER2 binding domain. In other words, the second domain is the second HER2 binding domain and the third domain is the first HER2 binding domain.

In some embodiments, the second domain or the third domain comprises the VL and VH of Trastuzumab, a known anti-HER2 antibody recognizing and binding to HER2 at its ECD2. The amino acid sequences of CDRs, VH and VL of Trastuzumab are shown in Table 1.

In some embodiments, the second domain or the third domain comprises the VL and VH of Pertuzumab, a known anti-HER2 antibody recognizing and binding to HER2 at its ECD4. The amino acid sequences of CDRs, VH and VL of Pertuzumab are shown in Table 1.

In some embodiments, the first HER2 binding domain comprises an LCDR1 comprising a sequence as shown in SEQ ID NO: 13, an LCDR2 comprising a sequence as shown in SEQ ID NO: 14, an LCDR3 comprising a sequence as shown in SEQ ID NO: 15 or SEQ ID NO: 97, and the VH2 comprises an HCDR1 comprising a sequence as shown in SEQ ID NO: 9, an HCDR2 comprising a sequence as shown in SEQ ID NO: 10, and an HCDR3 comprising a sequence as shown in SEQ ID NO: 11.

In some embodiments, the second HER2 binding domain comprises an LCDR1 comprising a sequence as shown in SEQ ID NO: 21, an LCDR2 comprising a sequence as shown in SEQ ID NO: 22, and an LCDR3 comprising a sequence as shown in SEQ ID NO: 23, and the VH3 comprises an HCDR1 comprising a sequence as shown in SEQ ID NO: 17, an HCDR2 comprising a sequence as shown in SEQ ID NO: 18, and an HCDR3 comprising a sequence as shown in SEQ ID NO: 19.

In some embodiments, the HER2 binding domain is modified to have reduced affinity to HER2 relative to the unmodified domain. In some embodiments, the HER2 binding domain is modified to comprise H107A on the VL according to IMGT numbering system. More details of H107A is available at, for example, Dionysos Slaga, et al, 2018. Science Translational Medicine, 10 (463), which is incorporated herein to its entirety. In some embodiments, the HER2 binding domain with low affinity comprises an LCDR1 comprising a sequence as shown in SEQ ID NO: 13, an LCDR2 comprising a sequence as shown in SEQ ID NO: 14, an LCDR3 comprising a sequence as shown in SEQ ID NO: 97, an HCDR1 comprising a sequence as shown in SEQ ID NO: 9, an HCDR2 comprising a sequence as shown in SEQ ID NO: 10, and an HCDR3 comprising a sequence as shown in SEQ ID NO: 11. In some embodiments, the HER2 binding domain with reduced affinity comprises a VL comprising a sequence as shown in SEQ ID NO: 95, and a VH comprising a sequence as shown in SEQ ID NO: 12.

In some embodiments, the HER2 binding domain is modified to facilitate cognate pairing relative to the unmodified domain. In some embodiments, the HER2 binding domain is modified to comprise A45L on the VH and W87Y on the VL, respectively, according to Kabat numbering system. In some embodiments, the HER2 binding domain comprises an LCDR1 comprising a sequence as shown in SEQ ID NO: 21, an LCDR2 comprising a sequence as shown in SEQ ID NO: 22, an LCDR3 comprising a sequence as shown in SEQ ID NO: 23, an HCDR1 comprising a sequence as shown in SEQ ID NO: 17, an HCDR2 comprising a sequence as shown in SEQ ID NO: 18, and an HCDR3 comprising a sequence as shown in SEQ ID NO: 19. In some embodiments, the HER2 binding domain comprises a VL comprising a sequence as shown in SEQ ID NO: 107, and a VH comprising a sequence as shown in SEQ ID NO: 106.

TABLE 1
CDR, VH and VL sequences of Trastuzumab (Tra) and Pertuzumab
(Per)

| Antibody | | CDR1 | CDR2 | CDR3 |
|---|---|---|---|---|
| Tra | HCDR | SEQ ID NO: 9 | SEQ ID NO: 10 | SEQ ID NO: 11 |
| | | GFNIKDTY | IYPTNGYT | SRWGGDGFYA |
| | | | | MDY |
| | VH | SEQ ID NO: 12 | | |
| | | EVQLVESGGGLVQPGGSLRLSCAASGFNIKDTYIHW | | |
| | | VRQAPGKGLEWVARIYPTNGYTRYADSVKGRFTISA | | |
| | | DTSKNTAYLQMNSLRAEDTAVYYCSRWGGDGFYA | | |
| | | MDYWGQGTLVTVSS | | |
| | LCDR | SEQ ID NO: 13 | SEQ ID NO: 14 | SEQ ID NO: 15 |
| | | QDVNTA | SAS | QQHYTTPPT |
| | VL | SEQ ID NO: 16 | | |
| | | DIQMTQSPSSLSASVGDRVTITCRASQDVNTAVAWY | | |
| | | QQKPGKAPKLLIYSASFLYSGVPSRFSGSRSGTDFTL | | |
| | | TISSLQPEDFATYYCQQHYTTPPTFGQGTKVEIK | | |
| Tra (low | HCDR | SEQ ID NO: 9 | SEQ ID NO: 10 | SEQ ID NO: 11 |
| affinity) | | GFNIKDTY | IYPTNGYT | SRWGGDGFYA |
| | | | | MDY |
| | VH | SEQ ID NO: 12 | | |
| | | EVQLVESGGGLVQPGGSLRLSCAASGFNIKDTYIHW | | |
| | | VRQAPGKGLEWVARIYPTNGYTRYADSVKGRFTISA | | |
| | | DTSKNTAYLQMNSLRAEDTAVYYCSRWGGDGFYA | | |
| | | MDYWGQGTLVTVSS | | |
| | LCDR | SEQ ID NO: 13 | SEQ ID NO: 14 | SEQ ID NO: 97 |
| | | QDVNTA | SAS | QQAYTTPPT |
| | VL | SEQ ID NO: 95 | | |
| | | DIQMTQSPSSLSASVGDRVTITCRASQDVNTAVAWY | | |
| | | QQKPGKAPKLLIYSASFLYSGVPSRFSGSRSGTDFTL | | |
| | | TISSLQPEDFATYYCQQAYTTPPTFGQGTKVEIK | | |
| Per | HCDR | SEQ ID NO: 17 | SEQ ID NO: 18 | SEQ ID NO: 19 |
| | | GFTFTDYT | VNPNSGGS | ARNLGPSFYFD |
| | | | | Y |
| | VH | SEQ ID NO: 20 | | |
| | | EVQLVESGGGLVQPGGSLRLSCAASGFTFTDYTMD | | |
| | | WVRQAPGKGAEWVADVNPNSGGSIYNQRFKGRFTL | | |
| | | SVDRSKNTLYLQMNSLRAEDTAVYYCARNLGPSFY | | |
| | | FDYWGQGTLVTVSS | | |
| | LCDR | SEQ ID NO: 21 | SEQ ID NO: 22 | SEQ ID NO: 23 |
| | | QDVSIG | SAS | QQYYIYPYT |
| | VL | SEQ ID NO: 24 | | |
| | | DIQMTQSPSSLSASVGDRVTITCKASQDVSIGVAWY | | |
| | | QQKPGKAPKLLIYSASYRYTGVPSRFSGSGSGTDFTL | | |
| | | TISSLQPEDFATYWCQQYYIYPYTFGQGTKVEIK | | |
| Per | HCDR | SEQ ID NO: 17 | SEQ ID NO: 18 | SEQ ID NO: 19 |
| (mutated) | | GFTFTDYT | VNPNSGGS | ARNLGPSFYFD |
| | | | | Y |
| | VH | SEQ ID NO: 106 | | |
| | | EVQLVESGGGLVQPGGSLRLSCAASGFTFTDYTMD | | |
| | | WVRQAPGKGLEWVADVNPNSGGSIYNQRFKGRFTL | | |
| | | SVDRSKNTLYLQMNSLRAEDTAVYYCARNLGPSFY | | |
| | | FDYWGQGTLVTVSS | | |
| | LCDR | SEQ ID NO: 21 | SEQ ID NO: 22 | SEQ ID NO: 23 |
| | | QDVSIG | SAS | QQYYIYPYT |

| | |
|---|---|
| VL | SEQ ID NO: 107 |
| | DIQMTQSPSSLSASVGDRVTITCKASQDVSIGVAWY |
| | QQKPGKAPKLLIYSASYRYTGVPSRFSGSGSGTDFT |
| | LTISSLQPEDFATYYCQQYYIYPYTFGQGTKVEIK |

In some embodiments, the first target is an immune stimulatory target. An immune stimulatory target is a target that, when activated, has a stimulatory effect on the immune system in the subject, for example, positively modulating cytokine secretion, NK cell activation, T cell proliferation, antibody production, or the like.

An important mechanism of bispecific antibody is to mediate the killing of immune cells, such as Natural Killer (NK) cells, T cells and macrophages. In recent years, with the deep understanding of the immune escape mechanism of cancer cells, the research on antibody drugs that activate immune cells, especially T cells, macrophages, and NK cells, has attracted much attention. For example, anti-CD3 bispecific antibodies can bind CD3 molecules on the surface of T cells and at the same time bind cancer cell surface antigens, so as to shorten the distance between cytotoxic T cells and cancer cells, guiding T cells to directly kill cancer cells, and no longer relying on the dual activation signal of T cells. This kind of bispecific antibody with immune stimulatory target has been considered as a major advantage in its mechanism of action.

In some embodiments, the immune stimulatory target is related to activating signal pathway of T cells, macrophages, or NK cells. Examples of immune stimulatory target include, without limitation, CD3, CD16, NKG2D, CD28, CD137 (4-1BB), OX40, CD27, GITR, ICOS, NKp46.

In some embodiments, the immune stimulatory target is selected from NKG2D, CD3 and CD16.

In some embodiments, the immune stimulatory target is NKG2D.

“NKG2D” refers to an activating receptor (transmembrane protein) belonging to the NKG2 family of C-type lectin-like receptors. NKG2D recognizes induced-self proteins from MIC and RAET1/ULBP families which appear on the surface of stressed, malignant transformed, and infected cells. NKG2D is a major recognition receptor for the detection and elimination of transformed and infected cells as its ligands are induced during cellular stress, either as a result of infection or genomic stress such as in cancer. In NK cells, NKG2D serves as an activating receptor, which is able to trigger cytotoxicity.

In some embodiments, the antigen binding domain of the anti-NKG2D antibody comprises an LCDR1 comprising a sequence as shown in SEQ ID NO: 29, an LCDR2 comprising a sequence as shown in SEQ ID NO: 30, an LCDR3 comprising a sequence as shown in SEQ ID NO: 31, an HCDR1 comprising a sequence as shown in SEQ ID NO: 25, an HCDR2 comprising a sequence as shown in SEQ ID NO: 26, and an HCDR3 comprising a sequence as shown in SEQ ID NO: 27. In some embodiments, the antigen binding domain of the anti-NKG2D antibody comprises a VL comprising a sequence as shown in SEQ ID NO: 32, and a VH comprising a sequence as shown in SEQ ID NO: 28. Exemplary anti-NKG2D antibody has the sequences as shown in Table 2.

In some embodiments, the antigen binding domain of the anti-NKG2D antibody comprises an LCDR1 comprising a sequence as shown in SEQ ID NO: 118, an LCDR2 comprising a sequence as shown in SEQ ID NO: 119, an LCDR3 comprising a sequence as shown in SEQ ID NO: 120, an HCDR1 comprising a sequence as shown in SEQ ID NO: 114, an HCDR2 comprising a sequence as shown in SEQ ID NO: 115, and an HCDR3 comprising a sequence as shown in SEQ ID NO: 116. In some embodiments, the antigen binding domain of the anti-NKG2D antibody comprises a VL comprising a sequence as shown in SEQ ID NO: 121, and a VH comprising a sequence as shown in SEQ ID NO: 117. Exemplary anti-NKG2D antibody has the sequences as shown in Table 2.

TABLE 2
CDR, VH and VL sequences of the exemplary anti-NKG2D
antibody

| Target | | CDR1 | CDR2 | CDR3 |
|---|---|---|---|---|
| NKG2D | HCDR | SEQ ID NO: 25 | SEQ ID NO: 26 | SEQ ID NO: 27 |
| | | GFTFSSYS | ISSSSSYI | ARGAPMGAAA |
| | | | | GWFDP |
| | VH | SEQ ID NO: 28 | | |
| | | EVQLVESGGGLVKPGGSLRLSCAASGFTFSSYSMNW | | |
| | | VRDAPGKGLEWVSSISSSSSYIYYADSVKGRFTISRD | | |
| | | NAKNSLYLQMNSLRAEDTAVYYCARGAPMGAAAG | | |
| | | WFDPWGQGTLVTVSS | | |
| | LCDR | SEQ ID NO: 29 | SEQ ID NO: 30 | SEQ ID NO: 31 |
| | | QGISSW | AAS | QQGVSFPRT |
| | VL | SEQ ID NO: 32 | | |
| | | DIQMTQSPSSVSASVGDRVTITCRASQGISSWLAWY | | |
| | | QKKPGKAPKLLIYAASSLQSGVPSRFSGSGSGTDFTL | | |
| | | TISSLQPEDFATYYCQQGVSFPRTFGGGTKVEIK | | |
| NKG2D | HCDR | SEQ ID NO: 114 | SEQ ID NO: 115 | SEQ ID NO: 116 |
| | | FTFSYGMH | FIRYDGSNKYY | AKDRFGYYLD |
| | | | ADSVKG | Y |
| | VH | SEQ ID NO: 117 | | |
| | | EVQLVESGGGVVQPGGSLRLSCAASGFTFSSYGMH | | |
| | | WVRQAPGKGLEWVAFIRYDGSNKYYADSVKGRFTI | | |
| | | SRDNSKNTKYLQMNSLRAEDTAVYYCAKDRFGYYL | | |
| | | DYWGQGTLVTVSS | | |
| | LCDR | SEQ ID NO: 118 | SEQ ID NO: 119 | SEQ ID NO: 120 |
| | | GGDDIETKSVH | DDDDRPS | QVWDDNNDE |
| | | | | WV |

| | |
|---|---|
| VL | SEQ ID NO: 121 |
| | QPVLTQPSSVSVAPGETARIPCGGDDIETKSVHWYQ |
| | QKPGQAPVLVIYDDDDRPSGIPERFFGSNSGNTATLS |
| | ISRVEAGDEADYYCQVWDDNNDEWVFGGGTQLTV |
| | L |

In some embodiments, the immune stimulatory target is CD3. “CD3” as used herein, refers to cluster of differentiation 3, which is a protein complex and T cell co-receptor that is involved in activating both the cytotoxic T cell (CD8⁺ naive T cells) and T helper cells (CD4⁺ naive T cells). It is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. Because CD3 is required for T cell activation, drugs (often monoclonal antibodies) that target it are being investigated as immunosuppressant therapies for cancers and other autoimmune diseases. New anticancer drug treatments are being developed based upon the CD3 T cell co-receptor, with molecules being designed for altering the co-stimulatory signal to help get the T-cell to recognize the cancer cell and become fully activated.

In some embodiments, the antigen binding domain of the anti-CD3 antibody comprises an LCDR1 comprising a sequence as shown in SEQ ID NO: 5, an LCDR2 comprising a sequence as shown in SEQ ID NO: 6, an LCDR3 comprising a sequence as shown in SEQ ID NO: 7, an HCDR1 comprising a sequence as shown in SEQ ID NO: 1, an HCDR2 comprising a sequence as shown in SEQ ID NO: 2, and an HCDR3 comprising a sequence as shown in SEQ ID NO: 3. In some embodiments, the antigen binding domain of the anti-CD3 antibody comprises a VL comprising a sequence as shown in SEQ ID NO: 8, and a VH comprising a sequence as shown in SEQ ID NO: 4.

In some embodiments, the antigen binding domain of the anti-CD3 antibody is modified to have reduced affinity to CD3 relative to the unmodified domain. In some embodiments, the antigen binding domain of the anti-CD3 antibody is modified to comprise S113T on the VH according to IMGT numbering system. More details of S113T is available at, for example, Dionysos Slaga, et al, 2018. Science Translational Medicine, 10 (463), which is incorporated herein to its entirety. In some embodiments, the CD3 binding domain with low affinity comprises an LCDR1 comprising a sequence as shown in SEQ ID NO: 5, an LCDR2 comprising a sequence as shown in SEQ ID NO: 6, an LCDR3 comprising a sequence as shown in SEQ ID NO: 7, an HCDR1 comprising a sequence as shown in SEQ ID NO: 1, an HCDR2 comprising a sequence as shown in SEQ ID NO: 2, and an HCDR3 comprising a sequence as shown in SEQ ID NO: 96. In some embodiments, the CD3 binding domain with low affinity comprises a VL comprising a sequence as shown in SEQ ID NO: 8, and a VH comprising a sequence as shown in SEQ ID NO: 124.

Exemplary anti-CD3 antibody has the sequences as shown in Table 3.

TABLE 3
CDR, VH and VL sequences of the exemplary anti-CD3 antibody

| Target | | CDR1 | CDR2 | CDR3 |
|---|---|---|---|---|
| CD3 | HCDR | SEQ ID NO: 1 | SEQ ID NO: 2 | SEQ ID NO: 3 |
| | | GFTFSTYA | IRSKYNNYAT | VRHGNFGNSY |
| | | | | VSWFAY |
| | VH | SEQ ID NO: 4 | | |
| | | EVQLVESGGGLVQPGGSLRLSCAASGFTESTYAMN | | |
| | | WVRQAPGKGLEWVGRIRSKYNNYATYYADSVKGR | | |
| | | FTISRDDSKNSLYLQMNSLRAEDTAVYYCVRHGNFG | | |
| | | NSYVSWFAYWGQGTLVTVSS | | |
| | LCDR | SEQ ID NO: 5 | SEQ ID NO: 6 | SEQ ID NO: 7 |
| | | TGAVTTSNY | GTN | ALWYSNLWV |
| | VL | SEQ ID NO: 8 | | |
| | | QAVVTQEPSLTVSPGGTVTLTCRSSTGAVTTSNYAN | | |
| | | WVQQKPGQAPRGLIGGTNKRAPWTPARFSGSLLGG | | |
| | | KAALTLSGAQAEDEADYYCALWYSNLWVFGGGTK | | |
| | | LTVL | | |
| CD3 (low | HCDR | SEQ ID NO: 1 | SEQ ID NO: 2 | SEQ ID NO: 96 |
| affinity) | | GFTFSTYA | IRSKYNNYAT | VRHGNFGNSY |
| | | | | VTWFAY |
| | VH | SEQ ID NO: 124 | | |
| | | EVQLVESGGGLVQPGGSLRLSCAASGFTFSTYAMN | | |
| | | WVRQAPGKGLEWVGRIRSKYNNYATYYADSVKGR | | |
| | | FTISRDDSKNSLYLQMNSLRAEDTAVYYCVRHGNFG | | |
| | | NSYVTWFAYWGQGTLVTVSS | | |
| | LCDR | SEQ ID NO: 5 | SEQ ID NO: 6 | SEQ ID NO: 7 |
| | | TGAVTTSNY | GTN | ALWYSNLWV |

| | |
|---|---|
| VL | SEQ ID NO: 8 |
| | QAVVTQEPSLTVSPGGTVTLTCRSSTGAVTTSNYAN |
| | WVQQKPGQAPRGLIGGTNKRAPWTPARFSGSLLGG |
| | KAALTLSGAQAEDEADYYCALWYSNLWVFGGGTK |
| | LTVL |

In some embodiments, the immune stimulatory target is CD16.

“CD16”, also known as FcγRIII, is a cluster of differentiation molecule found on the surface of natural killer cells, neutrophils, monocytes, macrophages, and certain T cells. The most well-researched membrane receptor implicated in triggering lysis by NK cells, CD16 is a molecule of the immunoglobulin superfamily (IgSF) involved in antibody-dependent cellular cytotoxicity (ADCC).

CD16 plays a significant role in early activation of natural killer (NK) cells following vaccination. In addition, CD16 downregulation represents a possible way to moderate NK cell responses and maintain immune homeostasis in both T cell and antibody-dependent signaling pathways. In a normal, healthy individual, cross-linking of CD16 (FcγRIII) by immune complexes induces antibody-dependent cellular cytotoxicity (ADCC) in NK cells. This pathway can also be targeted in cancerous or diseased cells by immunotherapy.

In some embodiments, the antigen binding domain of the anti-CD16 antibody comprises an LCDR1 comprising a sequence as shown in SEQ ID NO: 37, an LCDR2 comprising a sequence as shown in SEQ ID NO: 38, an LCDR3 comprising a sequence as shown in SEQ ID NO: 39, an HCDR1 comprising a sequence as shown in SEQ ID NO: 33, an HCDR2 comprising a sequence as shown in SEQ ID NO: 34, and an HCDR3 comprising a sequence as shown in SEQ ID NO: 35. In some embodiments, the antigen binding domain of the anti-CD16 antibody comprises a VL comprising a sequence as shown in SEQ ID NO: 40, and a VH comprising a sequence as shown in SEQ ID NO: 36. Exemplary anti-CD16 antibody has the sequences as shown in Table 4.

TABLE 4
CDR, VH and VL sequences of the exemplary
anti-CD16 antibody

| Target | | CDR1 | CDR2 | CDR3 |
|---|---|---|---|---|
| CD16 | HCDR | SEQ ID NO: 33 | SEQ ID NO: 34 | SEQ ID NO: 35 |
| | | GYTFTSYY | INPSGGST | ARGSAYYYDF |
| | | | | ADY |
| | VH | SEQ ID NO: 36 | | |
| | | QVQLVQSGAEVKKPGESLKVSCKASGYTFTSYYMH | | |
| | | WVRQAPGQGLEWMGIINPSGGSTSYAQKFQGRVTM | | |
| | | TRDTSTSTVYMELSSLRSEDTAVYYCARGSAYYYDF | | |
| | | ADYWGQGTLVTVSS | | |
| | LCDR | SEQ ID NO: 37 | SEQ ID NO: 38 | SEQ ID NO: 39 |
| | | NIGSKN | QDN | QVWDNYSVL |

| | |
|---|---|
| VL | SEQ ID NO: 40 |
| | SYVLTQPSSVSVAPGQTATISCGGHNIGSKNVHWYQ |
| | QRPGQSPVLVIYQDNKRPSGIPERFSGSNSGNTATLTI |
| | SGTQAMDEADYYCQVWDNYSVLFGGGTKLTVL |

### B. Modifications of the Engineered Antibodies

The engineered antibodies provided herein can further comprise one or more modifications useful for promoting the binding between the corresponding heavy chains and light chains, for example, to promote the binding between VH1 and VL1, or between VH2 and VL2. In some embodiments, non-native covalent bonds can be introduced, and/or electrostatic interactions can be introduced to the VH1-VL1 interface, or VH2-VL2 interface.

### Disulfide Bond Introduced in the VH-VL Regions

In some embodiments, one of the first domain (formed by association of VH1 and VL1) and the second domain (formed by association of VH2 and VL2) comprises a first non-native covalent bond.

In certain of these embodiments, the other one of the first domain and the second domain does not comprise a non-native covalent bond. Alternatively, the other one of the first domain and the second domain comprises a second non-native covalent bond that is distinct from the first non-native covalent bond. For example, the second non-native covalent bond is formed between a pair of two amino acid residues different from those two forming the first non-native covalent bond, or is formed between amino acid residues at a pair of positions different from those for the first non-native covalent bond.

In some embodiments, the first non-native covalent bond can be a non-native disulfide bond. In some embodiments, the first non-native disulfide bond is formed between two introduced cysteine residues. In such embodiments, at least one of the first domain and the second domain is a disulfide-stabilized Fv. Analysis on crystal structure of antibodies revealed that cysteine mutations could be introduced in some of the relatively conserved sequences at the VL-VH interface to form disulfide bonds between VL and VH, so they are covalently connected. Covalent bonds between VLs and VHs significantly improved stability of the antibodies. The initial dsFv (disulfide Fv) was constructed by introducing disulfide bonds to VH-VL interface via covalent interaction between cysteine residues in CDRs of each fragment respectively (see Glockshuber, R., Malia, M., Pfitzinger, I. and Pluckthun, A. A comparison of strategies to stabilize immunoglobulin Fv-fragments. (1990) Biochemistry, 29, 1362-1367.). Though activity of antibodies was not affected using this method, detailed structure information on the CDRs of the original antibodies was required for “customized” design to avoid interference with antigen-recognizing/binding capability of the CDRs, which made it difficult for the method to become a universal solution for construction of various antibodies. To ensure broad application of the method, it is crucial that only amino acids at selected sites in conserved FR are engaged in the construction of dsFv.

Since 1993, several paired sites for VH-VL covalent bond formation have been discovered, including VH44-VL100, VH105-VL43, VH100b-VL49, VH100-VL150, and vH101-vL46, etc. (see Reiter, Y., Brinkmann, R. J., Kreitman, R. J., etc. Stabilization of the Fv Fragments in Recombinant Immunotoxins by Disulfide Bonds Engineered Into Conserved Framework Regions. (1994) Biochemistry, 33, 5451-5459., Jung, S. H., Pastan, I. and Lee, B. Design of interchain disulfide bonds in the framework region of the Fv fragment of the monoclonal antibody B3. (1994) Proteins, Struc. Func. Genet., 19, 35-47., Glockshuber, R., Malia, M., Pfitzinger, I. and Plückthun, A. A comparison of strategies to stabilize immunoglobulin Fv-fragments. (1990) Biochemistry, 29, 1362-1367., and Zhu, Z., Presta, L. G., Zapata, G. and Carter, P. Remodeling domain interfaces to enhance heterodimer formation. (1997) Prot. Sci., 6, 781-788.). Among them VH44-VL100 and VH105-VL43 are, to different extent, superior to the others in many aspects such as protein expression level, mono rate, Tm and affinity etc., and are thus subjected to broader application.

In some embodiments, the VL2 and the VH2 of the engineered antibody are associated with a first non-native disulfide bond. In some embodiments, the first non-native disulfide bond is formed between two non-native cysteine residues in the VL2 and the VH2, respectively. In some embodiments, the two non-native cysteine residues are in the framework region (FR) of the VL2 and FR of the VH2, respectively. In some embodiments, the two non-native cysteine residues are in FR2 of the VL2 and an amino acid residue in FR4 of the VH2, respectively.

In some embodiments, the two non-native cysteine residues are at position 44 in the VH2 and position 100 in the VL2, or at position 105 in the VH2 and position 43 in the VL2, or at position 100 in the VH2 and position 49 in the VL2, or at position 100 in the VH2 and position 150 in the VL2. Unless otherwise specified, all position numberings in the present disclosure are according to Kabat index. In some embodiments, the non-native covalent bond is formed between introduced amino acid residues at position 44 in the VH2 and position 100 in the VL2, or cysteine residues at position 105 in the VH2 and position 43 in the VL2. In some embodiments, the non-native covalent bond is formed between cysteine residues at position 44 in the VH2 and position 100 in the VL2. In some embodiments, the two non-native cysteine residues are 100C in the VL2 and 44C in the VH2.

In some embodiments, the VL1 and the VH1 of the engineered antibody do not comprise any introduced non-native disulfide bond.

In some other embodiments, the VL1 and the VH1 of the engineered antibody are associated with a first non-native disulfide bond. In some embodiments, the first non-native disulfide bond is formed between two non-native cysteine residues in the VL1 and the VH1, respectively. In some embodiments, the two non-native cysteine residues are in the framework region (FR) of the VL1 and FR of the VH1, respectively. In some embodiments, the two non-native cysteine residues are in FR2 of the VL1 and an amino acid residue in FR4 of the VH1, respectively. In some embodiments, the two non-native cysteine residues are at position 44 in the VH1 and position 100 in the VL1, or at position 105 in the VH1 and position 43 in the VL1, or at position 100 in the VH1 and position 49 in the VL1, or at position 100 in the VH1 and position 150 in the VL1. Unless otherwise specified, all position numberings in the present disclosure are according to Kabat index. In some embodiments, the non-native covalent bond is formed between introduced amino acid residues at position 44 in the VH1 and position 100 in the VL1, or cysteine residues at position 105 in the VH1 and position 43 in the VL1. In some embodiments, the non-native covalent bond is formed between cysteine residues at position 44 in the VH1 and position 100 in the VL1. In some embodiments, the two non-native cysteine residues are 100C in the VL1 and 44C in the VH1. In some of these embodiments, the VL2 and the VH2 of the engineered antibody do not comprise any introduced non-native disulfide bond.

Substitution with Charged Amino Acids

The engineered antibodies provided herein are modified to introduce electrostatic interactions in the first domain (formed by association of VH1 and VL1) or in the second domain (formed by association of VH2 and VL2), such that the pairing between the VH1 and the VL1 or the pairing between the VH2 and the VL2 are facilitated or favored.

In some embodiments, the VL2 and the VH2 are modified to introduce two oppositely charged residues that promote electrostatic interactions between the VL2 and the VH2. In certain of these embodiments, the VL1 and the VH1 are not introduced with such oppositely charged residues.

In some embodiments, the VL1 and the VH1 are modified to introduce two oppositely charged residues that promote electrostatic interactions between the VL1 and the VH1. In certain of these embodiments, the VL2 and the VH2 are not introduced with such oppositely charged residues.

In some embodiments, the engineered antibodies provided herein are modified to introduce a first pair of two oppositely charged residues that promote electrostatic interactions between the VL2 and the VH2, as well as a second pair of two oppositely charged residues that promote electrostatic interactions between the VL1 and the VH1, with the proviso that the pairing between the VH1 and the VL2 and the pairing between the VH2 and the VL1 are discouraged, for example, due to electrostatic repulsion. For example, the charged residues introduced to the VL2 and the VH1 are like-charged residues, and/or the charged residues introduced to the VH2 and the VL1 are like-charged residues, such that mispairing between VL2 and VH1 or between VL1 and VH2 are discouraged.

In some embodiments, the two oppositely charged residues is composed of a positively charged residue and a negatively charged residue.

In certain embodiments, the first pair of two oppositely charged residues comprises a negatively charged residue in the VL2 and a positively charged residue in the VH2. In certain embodiments, the second pair of two oppositely charged residues comprises a positively charged residue in the VL1 and a negatively charged residue in the VH1.

In certain embodiments, the first pair of two oppositely charged residues comprises a positively charged residue in the VL2 and a negatively charged residue in the VH2. In certain embodiments, the second pair of two oppositely charged residues comprises a negatively charged residue in the VL1 and a positively charged residue in the VH1.

In some embodiments, the negatively charged amino acid is aspartic acid (D) or glutamic acid (E). In some embodiments, the positively charged amino acid is lysine (K), histidine (H) or arginine (R).

Strategies to enhancing the stably binding of corresponding VH and VL in the bispecific antibodies by introducing charged amino acids are well known in the art. Tan et al managed to influence stability of the scFv (single chain F variant) by adjusting amino acids at VH-VL interface based on their electrostatic properties (see Philip H. Tan, Brenda M. Sandmaier, Patrick S. Stayton. Contributions of a Highly Conserved VH VL Hydrogen Bonding Interaction to scFv Folding Stability and Refolding Efficiency. Biophys J. 1998 September; 75 (3): 1473-1482.). Later, Igawa et al adapted the method to modify scDb. Two pairs of Q39-Q38 in 4 V fragments respectively were replaced with amino acids with proper electrostatic charge to either promote or inhibit certain isoforms, in order to improve homogeneity of the product (see Igawa T, Tsunoda H, Kikuchi Y, etc. VH/VL interface engineering to promote selective expression and inhibit conformational isomerization of thrombopoietin receptor agonist single-chain diabody. Protein Eng Des Sel. 2010 August; 23 (8): 667-77., and WO2006106905A1). Gunasekaran et al at Amgen did further research on the method and engaged it in modification of the Fab arms of antibodies. Adjusting electrostatic steering at the CH1-CL interface, together with modifications at 38-39 of VH-VL, facilitated specific interaction between CH1-VH and CL-VL (see Gunasekaran K, Pentony M, Shen M, etc. Enhancing antibody Fc heterodimer formation through electrostatic steering effects: applications to bispecific molecules and monovalent IgG. J Biol Chem. 2010 Jun. 18; 285 (25): 19637-46.; and Liu Z, Leng EC2, Gunasekaran K3, etc. A novel antibody engineering strategy for making monovalent bispecific heterodimeric IgG antibodies by electrostatic steering mechanism. J Biol Chem. 2015 Mar. 20; 290 (12): 7535-62.). By these methods, each HC of the bispecific antibodies is able to interact with the corresponding LC, which results in bispecific antibodies that could bind to two antigens at the same time.

In some embodiments, the engineered antibodies provided herein modified electrostatic steering of selected regions in addition to introduction of non-native disulfide bonds, and by doing so managed to minimize unwanted non-specific interactions. The modification to introduce electrostatic interactions can improve pharmacokinetic properties of the engineered antibodies, help to remove obstacles in downstream development process, and increase probability of success in development of bispecific antibodies.

W103 of VH and P44 of VL are both at the side chain of the hydrophobic core and positioned in close proximity. Electrostatic interaction between W103-P44 was also examined during development of DICAD and found to be superior.

In some embodiments, the introduced charged residue in the VL1 or in the VL2 is in FR (e.g. FR2). In some embodiments, the introduced charged residue in the VH1 or VH2 is in FR (e.g. FR2).

In some embodiments, the first pair of two oppositely charged residues are introduced to replace Q38 in the VL2 and Q39 in the VH2, respectively, wherein numbering is according to the Kabat index. In some embodiments, the second pair of two oppositely charged residues are introduced to replace Q38 in the VL1 and Q39 in the VH1, respectively, wherein numbering is according to the Kabat index. In such embodiments, the charged residues introduced to the VL2 and the VH1 are like-charged residues, and/or the charged residues introduced to the VH2 and the VL1 are like-charged residues, such that mispairing between VL2 and VH1 or between VL1 and VH2 are discouraged.

In some embodiments, the first pair of two oppositely charged residues comprise Q38D in the VL2 and Q39K in the VH2, respectively, wherein numbering is according to the Kabat index. In some embodiments, the second pair of two oppositely charged residues comprise Q39D in the VH1 and Q40K in the VL1, respectively, wherein numbering is according to the Kabat index.

Introduction of positively or negatively charged amino acid into an antibody are known in the art.

In some embodiments, the charged amino acids are introduced in the VH2 and VL2 in addition to the introduced disulfide bond in the VH2 and VL2.

In some embodiments, the VH2 and the VL2 are derived from Trastuzumab. In some embodiments, the VH2 and the VL2 are derived from Tratuzumab with low affinity. In some embodiments, the cysteine residue is introduced at position 100 in the VL2 and position 44 in the VH2 to form the non-native disulfide bond, meanwhile two oppositely charged residues are introduced to replace Q38 in the VL2 and Q39 in the VH2, respectively, wherein numbering is according to the Kabat index. In some embodiments, the VH2 comprises mutations Q38D and G44C and the VL2 comprises mutations Q39D and Q100C. In some embodiments, the VH2 comprises a sequence as shown in SEQ ID NO: 90 and the VL2 comprises a sequence as shown in SEQ ID NO: 91. In some embodiments, the VH2 and the VL2 are derived from Tratuzumab with low affinity. In such embodiments, the VH2 comprises a sequence as shown in SEQ ID NO: 90 and the VL2 comprises a sequence as shown in SEQ ID NO: 98.

In some embodiments, the VH3 and the VL3 do not comprise any introduced non-native disulfide bond or pair of two oppositely charged amino acid residues.

In some embodiments, the VH3 and the VL3 are derived from Pertuzumab. In some embodiments, the VH3 comprises a sequence as shown in SEQ ID NO: 20 and the VL3 comprises a sequence as shown in SEQ ID NO: 24. In some embodiments, the VH3 comprises a sequence as shown in SEQ ID NO: 106 and the VL3 comprises a sequence as shown in SEQ ID NO: 107.

In some embodiments, the VH3 and the VL3 are derived from Trastuzumab. In some embodiments, the VH3 comprises a sequence as shown in SEQ ID NO: 12 and the VL3 comprises a sequence as shown in SEQ ID NO: 16. In some embodiments, the VH3 and the VL3 are derived from Trastuzumab with low affinity. In some embodiments, the VH3 comprises a sequence as shown in SEQ ID NO: 12 and the VL3 comprises a sequence as shown in SEQ ID NO: 95.

In some embodiments, the VH2 and the VL2 are derived from Pertuzumab. In some embodiments, the cysteine residue is introduced at position 100 in the VL2 and position 44 in the VH2 to form the non-native disulfide bond, meanwhile two oppositely charged residues are introduced to replace Q38 in the VL2 and Q39 in the VH2, respectively, wherein numbering is according to the Kabat index. In some embodiments, the VH2 comprises a sequence as shown in SEQ ID NO: 92 and the VL2 comprises a sequence as shown in SEQ ID NO: 93.

In some embodiments, the VH3 and the VL3 do not comprise any introduced non-native disulfide bond or pair of two oppositely charged amino acid residues. In some embodiments, the VH3 and the VL3 are derived from Tratuzumab. In some embodiments, the VH3 comprises a sequence as shown in SEQ ID NO: 12 and the VL3 comprises a sequence as shown in SEQ ID NO: 16. In some embodiments, the VH3 and the VL3 are derived from Trastuzumab with low affinity. In some embodiments, the VH3 comprises a sequence as shown in SEQ ID NO: 12 and the VL3 comprises a sequence as shown in SEQ ID NO: 95.

In some embodiments, the VH1 and VL1 of the engineered antibody do not comprise any introduced non-native disulfide bond or pair of two oppositely charged amino acid residues. In some embodiments, the VH1 and VL1 of the engineered antibody comprise introduced pair of two oppositely charged amino acid residues, but do not comprise any introduced non-native disulfide bond. In some embodiments, the VH1 and VL1 also comprise an introduced non-native disulfide bond and an introduced pair of two oppositely charged amino acid residues.

In some embodiments, the VH1 and the VL1 are derived from antigen-binding fragments targeting NKG2D, CD3 or CD16.

In some embodiments, the VH1 and the VL1 are derived from the variable regions in the exemplary anti-CD3 antibody as shown in Table 3. In some other embodiments, the VH1 and the VL1 comprise introduced pair of two oppositely charged amino acid residues. In some embodiments, two oppositely charged residues are introduced to replace Q40 in the VL1 and Q39 in the VH1, respectively. In some embodiments, the VL1 comprises the mutation Q40K and the VH1 comprises the mutation Q39D. In some embodiments, the VH1 comprises a sequence as shown in SEQ ID NO: 99 and the VL1 comprises a sequence as shown in SEQ ID NO: 100. In some embodiments, the anti-CD3 antibody has low affinity. In some embodiments, the VH1 comprises a sequence as shown in SEQ ID NO: 105 and the VL1 comprises a sequence as shown in SEQ ID NO: 100.

In some embodiments, the VH1 and the VL1 are derived from the variable regions in the exemplary anti-CD16 antibody as shown in Table 4. In some other embodiments, the VH1 and the VL1 comprise introduced pair of two oppositely charged amino acid residues. In some embodiments, the two oppositely charged residues are introduced to replace Q37 in the VL1 and Q39 in the VH1, respectively. In some embodiments, the VL1 comprises the mutation Q37K and the VH1 comprises the mutation Q39D. In some embodiments, the VH1 comprises a sequence as shown in SEQ ID NO: 108 and the VL1 comprises a sequence as shown in SEQ ID NO: 109.

In some embodiments, the VH1 and the VL1 are derived from the variable regions in one of the exemplary anti-NKG2D antibodies as shown in Table 2. In some other embodiments, the VH1 and the VL1 comprise introduced pair of two oppositely charged amino acid residues. In some embodiments, the two oppositely charged residues are introduced to replace Q37 in the VL1 and Q39 in the VH1, respectively. In some embodiments, the VL1 comprises the mutation Q37K and the VH1 comprises the mutation Q39D. In some embodiments, the VH1 comprises a sequence as shown in SEQ ID NO: 122 and the VL1 comprises a sequence as shown in SEQ ID NO: 123.

### Modifications in the Constant Regions

In some embodiments, in the engineered antibody provided herein, the second and the third polypeptide chain each further comprises a first dimerization domain and a second dimerization domain, respectively, that are associated to form a dimer.

In some embodiments, the first dimerization domain and the second dimerization domain comprise CH3 domain of IgG. In some embodiments, the first dimerization domain and the second dimerization domain further comprise a hinge region,

In some embodiments, the second polypeptide and the third polypeptide of the engineered antibody are covalently linked via a hinge region and form a knob-into-hole structure.

A knob-into-hole structure, also known as “protuberance-into-cavity” strategy which serves to engineer an interface between a first and second polypeptide for hetero-oligomerization.

In general, the preferred interface comprises at least a part of the CH3 domain of an antibody constant domain. “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. See U.S. Pat. No. 8,216,805, the disclosure of which is incorporated by reference in its entirety.

In some embodiments, the first dimerization domain and the second dimerization domain of the engineered antibody further comprise one or more mutations that facilitate heterodimerization. In some embodiments, the first dimerization domain comprises a first mutation, and the second dimerization domain comprises a second mutation.

In some embodiments, (a) the first mutation comprises T389W and/or S375C, and the second mutation comprises Y438V, T389S, L391A, and/or Y370C; (b) the first mutation comprises D427K and/or D377K, and the second mutation comprises K420D, and/or K440D; (c) the first mutation comprises D377K, E378K, and/or D427K, and the second mutation comprises K393E, K440D, and/or K470E; (d) the first mutation comprises S387H, and/or F436A, and the second mutation comprises Y370T, and/or T422F; (e) the first mutation comprises S387H, and/or T422F, and the second mutation comprises Y422T, and/or F436A; (f) the first mutation comprises K393D, and/or K440D, and the second mutation comprises E378K, and/or D427K; or (g) the first mutation comprises L372D, and/or L391E, and the second mutation comprises L372K, or T389K, wherein numbering is according to the Kabat index.

In some embodiments, the first mutation comprises T389W and the second domain comprises T389S, L391A and Y438V, wherein numbering is according to the Kabat index. In some embodiments, one of the first dimerization domain and the second dimerization domain, comprising the first mutation, comprises a sequence as shown in SEQ ID NO: 44, and the other of the first dimerization domain and the second dimerization domain, comprising the second mutation, comprises a sequence as shown in SEQ ID NO: 45.

In some embodiments, one of the first dimerization domain and the second dimerization domain comprises a sequence as shown in SEQ ID NO: 103, and the first dimerization domain and the second dimerization domain comprises a sequence as shown in SEQ ID NO: 104.

In some embodiments, the first dimerization domain and/or the second dimerization domain further comprise CH2 domain and/or hinge region of IgG. In some embodiments, the hinge region comprises a sequence as shown in SEQ ID NO: 43, 101 or 102.

In some embodiments, the third polypeptide of the engineered antibody disclosed herein further comprises a CH1 region, whose N-terminal is linked to the C-terminal of the VH3 of the engineered antibody. In some embodiments, the CH1 region comprises a sequence as shown in SEQ ID NO: 41.

In some embodiments, the fourth polypeptide of the engineered antibody disclosed herein further comprises a CL region, whose N-terminal is linked to the C-terminal of the VL3 of the engineered antibody. In some embodiments, the CL region comprises a sequence as shown in SEQ ID NO: 42.

In some embodiments, the engineered antibody provided herein is humanized. In some embodiments, the engineered antibody provided herein is a monoclonal antibody, a chimeric antibody or a labeled antibody.

### C. Illustrative Engineered Antibodies

In some embodiments, the engineered antibody disclosed herein comprises four polypeptide chains with the structures from N-terminal to C-terminal as following (see also, FIG. 2).
Chain 1: VL2 (HER2)-linker-VH1 (immune stimulatory target)
Chain 2: VL1 (immune stimulatory target)-linker-VH2 (HER2)-hinge-CH2-CH3
Chain 3: VH3 (HER2)-CH1-hinge-CH2-CH3
Chain 4: VL3 (HER2)-CL

### NKG2D×HER2 Multi-Specific Antibodies

In certain embodiments, the present disclosure provides certain engineered antibodies targeting NKG2D (the first target) and HER2 (the second target). In certain embodiments, such engineered antibodies are characterized in that: the VL1 and the VH1 associate to form the first domain capable of binding to NKG2D, the VL2 and the VH2 associate to form the first HER2 binding domain, and the VL3 and the VH3 associate to form the second HER2 binding domain.

In certain embodiments, the first domain is derived from the binding domain of an anti-NKG2D antibody, the second domain is derived from Trastuzumab, and the third domain is derived from Pertuzumab.

In certain embodiments, the engineered antibodies comprise the mutations in the variable regions or Fc regions listed in Table 5.

TABLE 5
Construct summary of the NKG2D × HER2 Multi-Specific Antibodies

| | Polypeptide | Domain Order from N | Mutation List | | | |
|---|---|---|---|---|---|---|
| Antibody | Chain | to C and Sequence | Domain 1 | Domain 2 | Domain 3 | Fc Region |
| 6A12 | 1 | VL2(HER2-Tra)-linker- | / | Q38D, | N/A | N/A |
| | (SEQ ID NO: | VH1(NKG2D) | | Q100C in | | |
| | 50) | VL2(HER2-Tra): SEQ ID | | VL2 | | |
| | | NO: 91; | | | | |
| | | VH1(NKG2D): SEQ ID | | | | |
| | | NO: 28; | | | | |
| | 2 | VL1(NKG2D)-linker- | / | Q39K, | N/A | T389W |
| | (SEQ ID NO: | VH2(HER2-Tra)-hinge- | | G44C in | | |
| | 51) | CH2-CH3 | | VH2 | | |
| | | VL1(NKG2D): SEQ ID | | | | |
| | | NO: 32; | | | | |
| | | VH2(HER2-Tra): SEQ ID | | | | |
| | | NO: 90; | | | | |
| | | CH2-CH3(Knob): SEQ | | | | |
| | | ID NO: 103 | | | | |
| | 3 | VH3(HER2-Per)-CH1- | N/A | N/A | / | Y438V, |
| | (SEQ ID NO: | hinge-CH2-CH3 | | | | T389S, |
| | 52) | VH3(HER2-Per): SEQ ID | | | | L391A |
| | | NO: 106; | | | | |
| | | CH2-CH3(Hole): SEQ ID | | | | |
| | | NO: 104 | | | | |
| | 4 | VL3(HER2-Per)-CL | N/A | N/A | / | N/A |
| | (SEQ ID NO: | VL3(HER2-Per): SEQ ID | | | | |
| | 53) | NO: 107; | | | | |
| 6A15 | 1 | VL2(HER2-Tra)-linker- | Q39D in | Q38D, | N/A | N/A |
| | (SEQ ID NO: | VH1(NKG2D) | VH2 | Q100C in | | |
| | 54) | VL2(HER2-Tra): SEQ ID | | VL2 | | |
| | | NO: 91; | | | | |
| | | VH1(NKG2D): SEQ ID | | | | |
| | | NO: 122; | | | | |
| | 2 | VL1(NKG2D)-linker- | Q37K in | Q39K, | N/A | T389W |
| | (SEQ ID NO: | VH2(HER2-Tra)-hinge- | VL2 | G44C in | | |
| | 55) | CH2-CH3 | | VH2 | | |
| | | VL1(NKG2D): SEQ ID | | | | |
| | | NO: 123; | | | | |
| | | VH2(HER2-Tra): SEQ ID | | | | |
| | | NO: 90; | | | | |
| | | CH2-CH3(Knob): SEQ | | | | |
| | | ID NO: 103 | | | | |
| | 3 | VH3(HER2-Per)-CH1- | N/A | N/A | / | Y438V, |
| | (SEQ ID NO: | hinge-CH2-CH3 | | | | T389S, |
| | 56) | VH3(HER2-Per): SEQ ID | | | | L391A |
| | | NO: 106; | | | | |
| | | CH2-CH3(Hole): SEQ ID | | | | |
| | | NO: 104 | | | | |
| | 4 | VL3(HER2-Per)-CL | N/A | N/A | / | N/A |
| | (SEQ ID NO: | VL3(HER2-Per): SEQ ID | | | | |
| | 57) | NO: 107; | | | | |
| 6A17 | 1 | VL2(HER2-Tra)-linker- | / | Q38D, | N/A | N/A |
| | (SEQ ID NO: | VH1(NKG2D) | | Q100C in | | |
| | 58) | VL2(HER2-Tra): SEQ ID | | VL2 | | |
| | | NO: 91; | | | | |
| | | VH1(NKG2D): SEQ ID | | | | |
| | | NO: 28; | | | | |
| | 2 | VL1(NKG2D)-linker- | / | Q39K, | N/A | T389W |
| | (SEQ ID NO: | VH2(HER2-Tra)-hinge- | | G44C in | | |
| | 59) | CH2-CH3 | | VH2 | | |
| | | VL1(NKG2D): SEQ ID | | | | |
| | | NO: 32; | | | | |
| | | VH2(HER2-Tra): SEQ ID | | | | |
| | | NO: 90; | | | | |
| | | CH2-CH3(Knob): SEQ | | | | |
| | | ID NO: 103 | | | | |
| | 3 | VH3(HER2-Per)-CH1- | N/A | N/A | / | Y438V, |
| | (SEQ ID NO: | hinge-CH2-CH3 | | | | T389S, |
| | 60) | VH3(HER2-Per): SEQ ID | | | | L391A |
| | | NO: 20; | | | | |
| | | CH2-CH3(Hole): SEQ ID | | | | |
| | | NO: 104 | | | | |
| | 4 | VL3(HER2-Per)-CL | N/A | N/A | / | N/A |
| | (SEQ ID NO: | VL3(HER2-Per): SEQ ID | | | | |
| | 61) | NO: 24; | | | | |

Notes:
1. “/”: No mutations.
2. “N/A (Not Applicable)”: the target domain (i.e. Domain 1, Domain 2, Domain 3 or Fc) is not present in the particular polypeptide chain (Chain 1, Chain 2, Chain 3 or Chain 4) of aforesaid antibodies.

In certain embodiments, the engineered antibody comprises the four polypeptide chains including Chain 1, Chain 2, Chain 3, and Chain 4, comprising the amino acid sequences of SEQ ID NOs: 50, 51, 52, and 53, respectively (which is also referred to herein as Antibody 6A12).

In certain embodiments, the engineered antibody comprise the four polypeptide chains including Chain 1, Chain 2, Chain 3, and Chain 4, comprising the amino acid sequences of SEQ ID NOs: 54, 55, 56, and 57, respectively (which is also referred to herein as Antibody 6A15).

In certain embodiments, the engineered antibody comprise the four polypeptide chains including Chain 1, Chain 2, Chain 3, and Chain 4, comprising the amino acid sequences of SEQ ID NOs: 58, 59, 60, and 61, respectively (which is also referred to herein as Antibody 6A17).

### CD3×HER2 Multi-Specific Antibodies

In certain embodiments, the present disclosure provides certain engineered antibodies targeting CD3 (the first target) and HER2 (the second target). In certain embodiments, such engineered antibodies are characterized in that: the VL1 and the VH1 associate to form the first domain capable of binding to CD3, the VL2 and the VH2 associate to form the second HER2 binding domain, and the VL3 and the VH3 associate to form the first HER2 binding domain.

In certain embodiments, the first domain is derived from the binding domain of an anti-CD3 antibody, the second domain is derived from Pertuzumab, and the third domain is derived from Trastuzumab.

In certain embodiments, the first domain is derived from the binding domain of an anti-CD3 antibody, the second domain is derived from Trastuzumab, and the third domain is derived from Pertuzumab.

In certain embodiments, the first domain is derived from the binding domain of an anti-CD3 antibody, both the second domain and the third domain are derived from Trastuzumab.

In certain embodiments, the first domain is derived from the binding domain of an anti-CD3 antibody, both the second domain and the third domain are derived from Trastuzumab with low affinity.

In certain embodiments, the engineered antibodies comprise the mutations in the variable regions or Fc regions listed in Table 6.

TABLE 6
Construct summary of the CD3 × HER2 Multi-Specific Antibodies

| | Polypeptide | Domains in Polypeptide | Mutation List | | | |
|---|---|---|---|---|---|---|
| Antibody | Chain | Chain from N to C | Domain 1 | Domain 2 | Domain 3 | Fc Region |
| 6A8 | Chain 1 | VL2(HER2-Tra)-linker- | Q39D in | Q38D, | N/A | N/A |
| | (SEQ ID NO: | VH1(CD3-low affinity) | VH1 | Q100C in | | |
| | 46) | VL2(HER2-Tra): SEQ | | VL2 | | |
| | | ID NO: 91; | | | | |
| | | VH1(CD3-low affinity): | | | | |
| | | SEQ ID NO: 105; | | | | |
| | Chain 2 | VL1(CD3-low affinity)- | Q40K in | Q39K, | N/A | T389W |
| | (SEQ ID NO: | linker-VH2(HER2-Tra)- | VL1 | G44C in | | |
| | 47) | hinge-CH2-CH3 | | VH2 | | |
| | | VL1(CD3-low affinity): | | | | |
| | | SEQ ID NO: 100; | | | | |
| | | VH2(HER2-Tra): SEQ | | | | |
| | | ID NO: 90; | | | | |
| | | CH2-CH3(Knob): SEQ | | | | |
| | | ID NO: 44 | | | | |
| | Chain 3 | VH3(HER2-Per)-CH1- | N/A | N/A | / | Y438V, |
| | (SEQ ID NO: | hinge-CH2-CH3 | | | | T389S, |
| | 48) | VH3(HER2-Per): SEQ | | | | L391A |
| | | ID NO: 106; | | | | |
| | | CH2-CH3(Hole): SEQ | | | | |
| | | ID NO: 45 | | | | |
| | Chain 4 | VL3(HER2-Per)-CL | N/A | N/A | / | N/A |
| | (SEQ ID NO: | VL3(HER2-Per): SEQ | | | | |
| | 49 | ID NO: 107; | | | | |
| 6A14 | Chain 1 | VL2(HER2-Tra)-linker- | Q39D in | Q38D, | N/A | N/A |
| | (SEQ ID NO: | VH1(CD3) | VH1 | Q100C in | | |
| | 82) | VL2(HER2-Tra): SEQ | | VL2 | | |
| | | ID NO: 91; | | | | |
| | | VH1(CD3): SEQ ID | | | | |
| | | NO: 99; | | | | |
| | Chain 2 | VL1(CD3)-linker- | Q40K in | Q39K, | N/A | T389W |
| | (SEQ ID NO: | VH2(HER2-Tra)-hinge- | VL1 | G44C in | | |
| | 47) | CH2-CH3 | | VH2 | | |
| | | VL1(CD3): SEQ ID NO: | | | | |
| | | 100; | | | | |
| | | VH2(HER2-Tra): SEQ | | | | |
| | | ID NO: 90; | | | | |
| | | CH2-CH3(Knob): SEQ | | | | |
| | | ID NO: 44 | | | | |
| | Chain 3 | VH3(HER2-Per)-CH1- | N/A | N/A | / | Y438V, |
| | (SEQ ID NO: | hinge-CH2-CH3 | | | | T389S, |
| | 48) | VH3(HER2-Per): SEQ | | | | L391A |
| | | ID NO: 106; | | | | |
| | | CH2-CH3(Hole): SEQ | | | | |
| | | ID NO: 45 | | | | |
| | Chain 4 | VL3(HER2-Per)-CL | N/A | N/A | / | N/A |
| | (SEQ ID NO: | VL3(HER2-Per): SEQ | | | | |
| | 49) | ID NO: 107; | | | | |
| 6A19 | Chain 1 | VL2(HER2-Per)-linker- | / | Q38D, | N/A | N/A |
| | (SEQ ID NO: | VH1(CD3) | | Q100C in | | |
| | 62) | VL2(HER2-Per): SEQ | | VL2 | | |
| | | ID NO: 93; | | | | |
| | | VH1(CD3): SEQ ID | | | | |
| | | NO: 4; | | | | |
| | Chain 2 | VL1(CD3)-linker- | / | K39Q, | N/A | T389W |
| | (SEQ ID NO: | VH2(HER2-Per)-hinge- | | G44C in | | |
| | 63) | CH2-CH3 | | VH2 | | |
| | | VL1(CD3): SEQ ID NO: | | | | |
| | | 8; | | | | |
| | | VH2(HER2-Per): SEQ | | | | |
| | | ID NO: 92; | | | | |
| | | CH2-CH3(Knob): SEQ | | | | |
| | | ID NO: 44 | | | | |
| | Chain 3 | VH3(HER2-Tra)-CH1- | N/A | N/A | / | Y438V, |
| | (SEQ ID NO: | hinge-CH2-CH3 | | | | T389S, |
| | 64) | VH3(HER2-Tra): SEQ | | | | L391A |
| | | ID NO: 12; | | | | |
| | | CH2-CH3(Hole): SEQ | | | | |
| | | ID NO: 45 | | | | |
| | Chain 4 | VL3(HER2-Tra)-CL | N/A | N/A | / | N/A |
| | (SEQ ID NO: | VL3(HER2-Tra): SEQ | | | | |
| | 65) | ID NO: 16; | | | | |
| 6A25 | Chain 1 | VL2(HER2-Tra-low | Q39D in | Q38D, | N/A | N/A |
| | (SEQ ID NO: | affinity)-linker- | VH1 | Q100C in | | |
| | 78) | VH1(CD3) | | VL2 | | |
| | | VL2(HER2-Tra-low | | | | |
| | | affinity): SEQ ID NO: | | | | |
| | | 98; | | | | |
| | | VH1(CD3): SEQ ID | | | | |
| | | NO: 99; | | | | |
| | Chain 2 | VL1(CD3)-linker- | Q40K in | Q39K, | N/A | T389W |
| | (SEQ ID NO: | VH2(HER2-Tra-low | VL1 | G44C in | | |
| | 79) | affinity)-hinge-CH2- | | VH2 | | |
| | | CH3 | | | | |
| | | VL1(CD3): SEQ ID NO: | | | | |
| | | 100; | | | | |
| | | VH2(HER2-Tra-low | | | | |
| | | affinity): SEQ ID NO: | | | | |
| | | 90; | | | | |
| | | CH2-CH3(Knob): SEQ | | | | |
| | | ID NO: 44 | | | | |
| | Chain 3 | VH3(HER2-Tra-low | N/A | N/A | / | Y438V, |
| | (SEQ ID NO: | affinity)-CH1-hinge- | | | | T389S, |
| | 80) | CH2-CH3 | | | | L391A |
| | | VH3(HER2-Tra-low | | | | |
| | | affinity): SEQ ID NO: | | | | |
| | | 12; | | | | |
| | | CH2-CH3(Hole): SEQ | | | | |
| | | ID NO: 45 | | | | |
| | Chain 4 | VL3(HER2-Tra-low | N/A | N/A | / | N/A |
| | (SEQ ID NO: | affinity)-CL | | | | |
| | 81) | VL3(HER2-Tra-low | | | | |
| | | affinity): SEQ ID NO: | | | | |
| | | 95; | | | | |
| 6A26 | Chain 1 | VL2(HER2-Tra)-linker- | Q39D in | Q38D, | N/A | N/A |
| | (SEQ ID NO: | VH1(CD3) | VH1 | Q100C in | | |
| | 82) | VL2(HER2-Tra): SEQ | | VL2 | | |
| | | ID NO: 91; | | | | |
| | | VH1(CD3): SEQ ID | | | | |
| | | NO: 99; | | | | |
| | Chain 2 | VL1(CD3)-linker- | Q40K in | Q39K, | N/A | T389W |
| | (SEQ ID NO: | VH2(HER2-Tra)-hinge- | VL1 | G44C in | | |
| | 83) | CH2-CH3 | | VH2 | | |
| | | VL1(CD3): SEQ ID NO: | | | | |
| | | 100; | | | | |
| | | VH2(HER2-Tra): SEQ | | | | |
| | | ID NO: 90; | | | | |
| | | CH2-CH3(Knob): SEQ | | | | |
| | | ID NO: 44 | | | | |
| | Chain 3 | VH3(HER2-Tra)-CH1- | N/A | N/A | / | Y438V, |
| | (SEQ ID NO: | hinge-CH2-CH3 | | | | T389S, |
| | 84) | VH3(HER2-Tra): SEQ | | | | L391A |
| | | ID NO: 12; | | | | |
| | | CH2-CH3(Hole): SEQ | | | | |
| | | ID NO: 45 | | | | |
| | Chain 4 | VL3(HER2-Tra)-CL | N/A | N/A | / | N/A |
| | (SEQ ID NO: | VL3(HER2-Tra): SEQ | | | | |
| | 85) | ID NO: 16; | | | | |

Notes:
1. “/”: No mutations.
2. “N/A (Not Applicable)”: the target domain (i.e. Domain 1, Domain 2, Domain 3 or Fc) is not present in the particular polypeptide chain (Chain 1, Chain 2, Chain 3 or Chain 4) of aforesaid antibodies.

In certain embodiments, the engineered antibody comprise the four polypeptide chains including Chain 1, Chain 2, Chain 3, and Chain 4, comprising the amino acid sequences of SEQ ID NOs: 46, 47, 48, and 49, respectively (which is also referred to herein as Antibody 6A8).

In certain embodiments, the engineered antibody comprise the four polypeptide chains including Chain 1, Chain 2, Chain 3, and Chain 4, comprising the amino acid sequences of SEQ ID NOs: 82, 47, 48 and 49, respectively (which is also referred to herein as Antibody 6A14).

In certain embodiments, the engineered antibody comprise the four polypeptide chains including Chain 1, Chain 2, Chain 3, and Chain 4, comprising the amino acid sequences of SEQ ID NOs: 62, 63, 64, and 65, respectively (which is also referred to herein as Antibody 6A19).

In certain embodiments, the engineered antibody comprise the four polypeptide chains including Chain 1, Chain 2, Chain 3, and Chain 4, comprising the amino acid sequences of SEQ ID NOs: 78, 79, 80, and 81, respectively (which is also referred to herein as Antibody 6A25.

In certain embodiments, the engineered antibody comprise the four polypeptide chains including Chain 1, Chain 2, Chain 3, and Chain 4, comprising the amino acid sequences of SEQ ID NOs: 82, 83, 84, and 85, respectively (which is also referred to herein as Antibody 6A26).

### CD16×HER2 Multi-Specific Antibodies

In certain embodiments, the present disclosure provides certain engineered antibodies targeting CD16 (the first target) and HER2 (the second target). In certain embodiments, such engineered antibodies are characterized in that: the VL1 and the VH1 associate to form the first domain capable of binding to CD16, the VL2 and the VH2 associate to form the second HER2 binding domain, and the VL3 and the VH3 associate to form the first HER2 binding domain.

In certain embodiments, the first domain is derived from the binding domain of an anti-CD16 antibody, the second domain is derived from Pertuzumab, and the third domain is derived from Trastuzumab.

In certain embodiments, the first domain is derived from the binding domain of an anti-CD16 antibody, the second domain is derived from Trastuzumab, and the third domain is derived from Pertuzumab.

In certain embodiments, the engineered antibodies comprise the mutations in the variable regions or Fc regions listed in Table 7. In certain embodiments, the engineered antibodies comprise the sequences listed in Table 7.

TABLE 7
Construct summary of the CD16 × HER2 Multi-Specific Antibodies

| | Polypeptide | Domain Order from N to | Mutation List | | | |
|---|---|---|---|---|---|---|
| Antibody | Chain | C and Sequence | Domain 1 | Domain 2 | Domain 3 | Fc Region |
| 6A18 | 1 | VL2(HER2-Tra)-linker- | Q39D in | Q38D, | N/A | N/A |
| | (SEQ ID NO: | VH1(CD16) | VH1 | Q100C in | | |
| | 66) | VL2(HER2-Tra): SEQ ID | | VL2 | | |
| | | NO: 91; | | | | |
| | | VH1(CD16): SEQ ID NO: | | | | |
| | | 108; | | | | |
| | 2 | VL1(CD16)-linker- | Q37K in | Q39K, | N/A | T389W |
| | (SEQ ID NO: | VH2(HER2-Tra)-hinge- | VL1 | G44C in | | |
| | 67) | CH2-CH3 | | VH2 | | |
| | | VL1(CD16): SEQ ID NO: | | | | |
| | | 109; | | | | |
| | | VH2(HER2-Tra): SEQ ID | | | | |
| | | NO: 90; | | | | |
| | | CH2-CH3(Knob): SEQ ID | | | | |
| | | NO: 44 | | | | |
| | 3 | VH3(HER2-Per)-CH1- | N/A | N/A | / | Y438V, |
| | (SEQ ID NO: | hinge-CH2-CH3 | | | | T389S, |
| | 68) | VH3(HER2-Per): SEQ ID | | | | L391A |
| | | NO: 20; | | | | |
| | | CH2-CH3(Hole): SEQ ID | | | | |
| | | NO: 45 | | | | |
| | 4 | VL3(HER2-Per)-CL | N/A | N/A | / | N/A |
| | (SEQ ID NO: | VL3(HER2-Per): SEQ ID | | | | |
| | 69) | NO: 24; | | | | |
| 6A23 | 1 | VL2(HER2-Per)-linker- | / | Q38D, | N/A | N/A |
| | (SEQ ID NO: | VH1(CD16) | | Q100C in | | |
| | 74) | VL2(HER2-Per): SEQ ID | | VL2 | | |
| | | NO: 93; | | | | |
| | | VH1(CD16): SEQ ID NO: | | | | |
| | | 36; | | | | |
| | 2 | VL1(CD16)-linker- | / | K39Q, | N/A | T389W |
| | (SEQ ID NO: | VH2(HER2-Per)-hinge- | | G44C in | | |
| | 75) | CH2-CH3 | | VH2 | | |
| | | VL1(CD16): SEQ ID NO: | | | | |
| | | 40; | | | | |
| | | VH2(HER2-Per): SEQ ID | | | | |
| | | NO: 92; | | | | |
| | | CH2-CH3(Knob): SEQ ID | | | | |
| | | NO: 44 | | | | |
| | 3 | VH3(HER2-Tra)-CH1- | N/A | N/A | / | Y438V, |
| | (SEQ ID NO: | hinge-CH2-CH3 | | | | T389S, |
| | 76) | VH3(HER2-Tra): SEQ ID | | | | L391A |
| | | NO: 12; | | | | |
| | | CH2-CH3(Hole): SEQ ID | | | | |
| | | NO: 45 | | | | |
| | 4 | VL3(HER2-Tra)-CL | N/A | N/A | / | N/A |
| | (SEQ ID NO: | VL3(HER2-Tra): SEQ ID | | | | |
| | 77) | NO: 16; | | | | |

Notes:
1. “/”: No mutations.
2. “N/A (Not Applicable)”: the target domain (i.e. Domain 1, Domain 2, Domain 3 or Fc) is not present in the particular polypeptide chain (Chain 1, Chain 2, Chain 3 or Chain 4) of aforesaid antibodies.

In certain embodiments, the engineered antibody comprise the four polypeptide chains including Chain 1, Chain 2, Chain 3, and Chain 4, comprising the amino acid sequences of SEQ ID NOs: 66, 67, 68, and 69, respectively (which is also referred to herein as Antibody 6A18).

In certain embodiments, the engineered antibody comprise the four polypeptide chains including Chain 1, Chain 2, Chain 3, and Chain 4, comprising the amino acid sequences of SEQ ID NOs: 74, 75, 76, and 77, respectively (which is also referred to herein as Antibody 6A23).

TABLE 8
Sequences of reference antibodies

| Antibody | Chain | |
|---|---|---|
| name | number | sequence |
| 6A8 | Chain 1 | SEQ ID NO: 46 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVQPGGSLRLSCAASGFTFS |
| | | TYAMNWVRDAPGKGLEWVGRIRSKYNNYATYYA |
| | | DSVKGRFTISRDDSKNSLYLQMNSLRAEDTAVYYC |
| | | VRHGNFGNSYVTWFAYWGQGTLVTVSS |
| | Chain 2 | SEQ ID NO: 47 (same as SEQ ID NOs: 79 and 83) |
| | | QAVVTQEPSLTVSPGGTVTLTCRSSTGAVTTSNYA |
| | | NWVQKKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFNIKDTYIHWVRKAPGKCLEWVARIYPTNGYT |
| | | RYADSVKGRFTISADTSKNTAYLQMNSLRAEDTAV |
| | | YYCSRWGGDGFYAMDYWGQGTLVTVSSASEPKSS |
| | | DKTHTCPPCPAPPVAGPSVFLFPPKPKDTLMISRTPE |
| | | VTCVVVDVSQEDPEVKFNWYVDGVEVHNAKTKP |
| | | REEQFNSTYRVVSVLTVLHQDWLNGKEYKCKVSN |
| | | KGLPSSIEKTISKAKGQPREPQVYTLPPSRDELTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSP |
| | Chain 3 | SEQ ID NO: 48 |
| | | EVQLVESGGGLVQPGGSLRLSCAASGFTFTDYTMD |
| | | WVRQAPGKGLEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPPVAGPSVFLFPP |
| | | KPKDTLMISRTPEVTCVVVDVSQEDPEVKFNWYV |
| | | DGVEVHNAKTKPREEQFNSTYRVVSVLTVLHQDW |
| | | LNGKEYKCKVSNKGLPSSIEKTISKAKGQPREPQVY |
| | | TLPPSRDELTKNQVSLSCAVKGFYPSDIAVEWESNG |
| | | QPENNYKTTPPVLDSDGSFFLVSKLTVDKSRWQQG |
| | | NVFSCSVMHEALHNRFTQKSLSLSP |
| | Chain 4 | SEQ ID NO: 49 (same as SEQ ID NOs: 53, 57 and 73) |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYYCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A12 | Chain 1 | SEQ ID NO: 50 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVKPGGSLRLSCAASGFTFS |
| | | SYSMNWVRDAPGKGLEWVSSISSSSSYIYYADSVK |
| | | GRFTISRDNAKNSLYLQMNSLRAEDTAVYYCARG |
| | | APMGAAAGWFDPWGQGTLVTVSS |
| | Chain 2 | SEQ ID NO: 51 |
| | | DIQMTQSPSSVSASVGDRVTITCRASQGISSWLAWY |
| | | QKKPGKAPKLLIYAASSLQSGVPSRFSGSGSGTDFT |
| | | LTISSLQPEDFATYYCQQGVSFPRTFGGGTKVEIKR |
| | | TVAAEVQLVESGGGLVQPGGSLRLSCAASGFNIKD |
| | | TYIHWVRKAPGKCLEWVARIYPTNGYTRYADSVK |
| | | GRFTISADTSKNTAYLQMNSLRAEDTAVYYCSRW |
| | | GGDGFYAMDYWGQGTLVTVSSASEPKSSDKTHTC |
| | | PPCPAPELLAGPDVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALPL |
| | | PEEKTISKAKGQPREPQVYTLPPSRDELTKNQVSLW |
| | | CLVKGFYPSDIAVEWESNGQPENNYKTTPPVLDSD |
| | | GSFFLYSKLTVDKSRWQQGNVFSCSVMHEALHNH |
| | | YTQKSLSLSP |
| | Chain 3 | SEQ ID NO: 52 |
| | | EVQLVESGGGLVQPGGSLRLSCAASGFTFTDYTMD |
| | | WVRQAPGKGLEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLAGPDVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPLPEEKTISKAKGQPREPQV |
| | | YTLPPSRDELTKNQVSLSCAVKGFYPSDIAVEWESN |
| | | GQPENNYKTTPPVLDSDGSFFLVSKLTVDKSRWQQ |
| | | GNVFSCSVMHEALHNRFTQKSLSLSP |
| | Chain 4 | SEQ ID NO: 49 |
| | | (same as SEQ ID NOs: 53, 57 and 73) |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYYCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A14 | Chain 1 | SEQ ID NO: 82 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVQPGGSLRLSCAASGFTFS |
| | | TYAMNWVRDAPGKGLEWVGRIRSKYNNYATYYA |
| | | DSVKGRFTISRDDSKNSLYLQMNSLRAEDTAVYYC |
| | | VRHGNFGNSYVSWFAYWGQGTLVTVSS |
| | Chain 2 | SEQ ID NO: 47 (same as SEQ ID NOs: 79 and 83) |
| | | QAVVTQEPSLTVSPGGTVTLTCRSSTGAVTTSNYA |
| | | NWVQKKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFNIKDTYIHWVRKAPGKCLEWVARIYPTNGYT |
| | | RYADSVKGRFTISADTSKNTAYLQMNSLRAEDTAV |
| | | YYCSRWGGDGFYAMDYWGQGTLVTVSSASEPKSS |
| | | DKTHTCPPCPAPPVAGPSVFLFPPKPKDTLMISRTPE |
| | | VTCVVVDVSQEDPEVKFNWYVDGVEVHNAKTKP |
| | | REEQFNSTYRVVSVLTVLHQDWLNGKEYKCKVSN |
| | | KGLPSSIEKTISKAKGQPREPQVYTLPPSRDELTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSP |
| | Chain 3 | SEQ ID NO: 48 |
| | | EVQLVESGGGLVQPGGSLRLSCAASGFTFTDYTMD |
| | | WVRQAPGKGLEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPPVAGPSVFLFPP |
| | | KPKDTLMISRTPEVTCVVVDVSQEDPEVKFNWYV |
| | | DGVEVHNAKTKPREEQFNSTYRVVSVLTVLHQDW |
| | | LNGKEYKCKVSNKGLPSSIEKTISKAKGQPREPQVY |
| | | TLPPSRDELTKNQVSLSCAVKGFYPSDIAVEWESNG |
| | | QPENNYKTTPPVLDSDGSFFLVSKLTVDKSRWQQG |
| | | NVFSCSVMHEALHNRFTQKSLSLSP |
| | Chain 4 | SEQ ID NO: 49 (same as SEQ ID NOs: 53, 57 and 73) |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYYCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A15 | Chain 1 | SEQ ID NO: 54 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGVVQPGGSLRLSCAASGFTFS |
| | | SYGMHWVRDAPGKGLEWVAFIRYDGSNKYYADS |
| | | VKGRFTISRDNSKNTKYLQMNSLRAEDTAVYYCA |
| | | KDRFGYYLDYWGQGTLVTVSS |
| | Chain 2 | SEQ ID NO: 55 |
| | | QPVLTQPSSVSVAPGETARIPCGGDDIETKSVHWYQ |
| | | KKPGQAPVLVIYDDDDRPSGIPERFFGSNSGNTATL |
| | | SISRVEAGDEADYYCQVWDDNNDEWVFGGGTQLT |
| | | VLRTVAAEVQLVESGGGLVQPGGSLRLSCAASGEN |
| | | IKDTYIHWVRKAPGKCLEWVARIYPTNGYTRYAD |
| | | SVKGRFTISADTSKNTAYLQMNSLRAEDTAVYYCS |
| | | RWGGDGFYAMDYWGQGTLVTVSSASEPKSSDKT |
| | | HTCPPCPAPELLAGPDVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKA |
| | | LPLPEEKTISKAKGQPREPQVYTLPPSRDELTKNQV |
| | | SLWCLVKGFYPSDIAVEWESNGQPENNYKTTPPVL |
| | | DSDGSFFLYSKLTVDKSRWQQGNVFSCSVMHEAL |
| | | HNHYTQKSLSLSP |
| | Chain 3 | SEQ ID NO: 56 |
| | | EVQLVESGGGLVQPGGSLRLSCAASGFTFTDYTMD |
| | | WVRQAPGKGLEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLAGPDVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPLPEEKTISKAKGQPREPQV |
| | | YTLPPSRDELTKNQVSLSCAVKGFYPSDIAVEWESN |
| | | GQPENNYKTTPPVLDSDGSFFLVSKLTVDKSRWQQ |
| | | GNVFSCSVMHEALHNRFTQKSLSLSP |
| | Chain 4 | SEQ ID NO: 49 (same as SEQ ID NOs: 53, 57 and 73) |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYYCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A17 | Chain 1 | SEQ ID NO: 58 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVKPGGSLRLSCAASGFTFS |
| | | SYSMNWVRDAPGKGLEWVSSISSSSSYIYYADSVK |
| | | GRFTISRDNAKNSLYLQMNSLRAEDTAVYYCARG |
| | | APMGAAAGWFDPWGQGTLVTVSS |
| | Chain 2 | SEQ ID NO: 59 |
| | | DIQMTQSPSSVSASVGDRVTITCRASQGISSWLAWY |
| | | QKKPGKAPKLLIYAASSLQSGVPSRFSGSGSGTDFT |
| | | LTISSLQPEDFATYYCQQGVSFPRTFGGGTKVEIKR |
| | | TVAAEVQLVESGGGLVQPGGSLRLSCAASGFNIKD |
| | | TYIHWVRKAPGKCLEWVARIYPTNGYTRYADSVK |
| | | GRFTISADTSKNTAYLQMNSLRAEDTAVYYCSRW |
| | | GGDGFYAMDYWGQGTLVTVSSASEPKSSDKTHTC |
| | | PPCPAPELLAGPDVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALPL |
| | | PEEKTISKAKGQPREPQVYTLPPSRDELTKNQVSLW |
| | | CLVKGFYPSDIAVEWESNGQPENNYKTTPPVLDSD |
| | | GSFFLYSKLTVDKSRWQQGNVFSCSVMHEALHNH |
| | | YTQKSLSLSP |
| | Chain 3 | SEQ ID NO: 60 |
| | | EVQLVESGGGLVQPGGSLRLSCAASGFTFTDYTMD |
| | | WVRQAPGKGAEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLAGPDVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPLPEEKTISKAKGQPREPQV |
| | | YTLPPSRDELTKNQVSLSCAVKGFYPSDIAVEWESN |
| | | GQPENNYKTTPPVLDSDGSFFLVSKLTVDKSRWQQ |
| | | GNVFSCSVMHEALHNRFTQKSLSLSP |
| | Chain 4 | SEQ ID NO: 61 |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYWCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A18 | Chain 1 | SEQ ID NO: 66 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAQVQLVQSGAEVKKPGESLKVSCKASGYTF |
| | | TSYYMHWVRDAPGQGLEWMGIINPSGGSTSYAQK |
| | | FQGRVTMTRDTSTSTVYMELSSLRSEDTAVYYCAR |
| | | GSAYYYDFADYWGQGTLVTVSS |
| | Chain 2 | SEQ ID NO: 67 |
| | | SYVLTQPSSVSVAPGQTATISCGGHNIGSKNVHWY |
| | | QKRPGQSPVLVIYQDNKRPSGIPERFSGSNSGNTAT |
| | | LTISGTQAMDEADYYCQVWDNYSVLFGGGTKLTV |
| | | LRTVAAEVQLVESGGGLVQPGGSLRLSCAASGENI |
| | | KDTYIHWVRKAPGKCLEWVARIYPTNGYTRYADS |
| | | VKGRFTISADTSKNTAYLQMNSLRAEDTAVYYCSR |
| | | WGGDGFYAMDYWGQGTLVTVSSASEPKSSDKTHT |
| | | CPPCPAPPVAGPSVFLFPPKPKDTLMISRTPEVTCVV |
| | | VDVSQEDPEVKFNWYVDGVEVHNAKTKPREEQFN |
| | | STYRVVSVLTVLHQDWLNGKEYKCKVSNKGLPSSI |
| | | EKTISKAKGQPREPQVYTLPPSRDELTKNQVSLWC |
| | | LVKGFYPSDIAVEWESNGQPENNYKTTPPVLDSDG |
| | | SFFLYSKLTVDKSRWQQGNVFSCSVMHEALHNHY |
| | | TQKSLSLSP |
| | Chain 3 | SEQ ID NO: 68 |
| | | EVQLVESGGGLVQPGGSLRLSCAASGFTFTDYTMD |
| | | WVRQAPGKGAEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPPVAGPSVFLFPP |
| | | KPKDTLMISRTPEVTCVVVDVSQEDPEVKFNWYV |
| | | DGVEVHNAKTKPREEQFNSTYRVVSVLTVLHQDW |
| | | LNGKEYKCKVSNKGLPSSIEKTISKAKGQPREPQVY |
| | | TLPPSRDELTKNQVSLSCAVKGFYPSDIAVEWESNG |
| | | QPENNYKTTPPVLDSDGSFFLVSKLTVDKSRWQQG |
| | | NVFSCSVMHEALHNRFTQKSLSLSP |
| | Chain 4 | SEQ ID NO: 69 |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYWCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A19 | Chain 1 | SEQ ID NO: 62 |
| | | DIQMTQSPSSLSASVGDRVTITCKASQDVSIGVAWY |
| | | QDKPGKAPKLLIYSASYRYTGVPSRFSGSGSGTDFT |
| | | LTISSLQPEDFATYYCQQYYIYPYTFGCGTKVEIKR |
| | | TVAAEVQLVESGGGLVQPGGSLRLSCAASGFTFST |
| | | YAMNWVRQAPGKGLEWVGRIRSKYNNYATYYAD |
| | | SVKGRFTISRDDSKNSLYLQMNSLRAEDTAVYYCV |
| | | RHGNFGNSYVSWFAYWGQGTLVTVSS |
| | Chain 2 | SEQ ID NO: 63 |
| | | QAVVTQEPSLTVSPGGTVTLTCRSSTGAVTTSNYA |
| | | NWVQQKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFTFTDYTMDWVRKAPGKCLEWVADVNPNSG |
| | | GSIYNQRFKGRFTLSVDRSKNTLYLQMNSLRAEDT |
| | | AVYYCARNLGPSFYFDYWGQGTLVTVSSDKTHTC |
| | | PPCPAPPVAGPSVFLFPPKPKDTLMISRTPEVTCVVV |
| | | DVSQEDPEVKFNWYVDGVEVHNAKTKPREEQFNS |
| | | TYRVVSVLTVLHQDWLNGKEYKCKVSNKGLPSSIE |
| | | KTISKAKGQPREPQVYTLPPSRDELTKNQVSLWCL |
| | | VKGFYPSDIAVEWESNGQPENNYKTTPPVLDSDGS |
| | | FFLYSKLTVDKSRWQQGNVFSCSVMHEALHNHYT |
| | | QKSLSLSP |
| | Chain 3 | SEQ ID NO: 64 (same as SEQ ID NOs: 76, 80 and 84) |
| | | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPPVAGPSVFLF |
| | | PPKPKDTLMISRTPEVTCVVVDVSQEDPEVKFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSRDELTKNQVSLSCAVKGFYPSDIAVEWES |
| | | NGQPENNYKTTPPVLDSDGSFFLVSKLTVDKSRWQ |
| | | QGNVFSCSVMHEALHNRFTQKSLSLSP |
| | Chain 4 | SEQ ID NO: 65 (same as SEQ ID NOs: 77, 85 and 89) |
| | | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQHYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 6A21 | Chain 1 | SEQ ID NO: 70 |
| | | QAVVTQEPSLTVSPGGTVTLTCRSSTGAVTTSNYA |
| | | NWVQQKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFNIKDTYIHWVRKAPGKCLEWVARIYPTNGYT |
| | | RYADSVKGRFTISADTSKNTAYLQMNSLRAEDTAV |
| | | YYCSRWGGDGFYAMDYWGQGTLVTVSS |
| | Chain 2 | SEQ ID NO: 71 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVQPGGSLRLSCAASGFTFS |
| | | TYAMNWVRQAPGKGLEWVGRIRSKYNNYATYYA |
| | | DSVKGRFTISRDDSKNSLYLQMNSLRAEDTAVYYC |
| | | VRHGNFGNSYVSWFAYWGQGTLVTVSSASEPKSS |
| | | DKTHTCPPCPAPPVAGPSVFLFPPKPKDTLMISRTPE |
| | | VTCVVVDVSQEDPEVKFNWYVDGVEVHNAKTKP |
| | | REEQFNSTYRVVSVLTVLHQDWLNGKEYKCKVSN |
| | | KGLPSSIEKTISKAKGQPREPQVYTLPPSRDELTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSP |
| | Chain 3 | SEQ ID NO: 72 |
| | | EVQLVESGGGLVQPGGSLRLSCAASGFTFTDYTMD |
| | | WVRQAPGKGLEWVADVNPNSGGSIYNQRFKGRFT |
| | | LSVDRSKNTLYLQMNSLRAEDTAVYYCARNLGPSF |
| | | YFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPPVAGPSVFLFPP |
| | | KPKDTLMISRTPEVTCVVVDVSQEDPEVKFNWYV |
| | | DGVEVHNAKTKPREEQFNSTYRVVSVLTVLHQDW |
| | | LNGKEYKCKVSNKGLPSSIEKTISKAKGQPREPQVY |
| | | TLPPSRDELTKNQVSLSCAVKGFYPSDIAVEWESNG |
| | | QPENNYKTTPPVLDSDGSFFLVSKLTVDKSRWQQG |
| | | NVFSCSVMHEALHNRFTQKSLSLSP |
| | Chain 4 | SEQ ID NO: 49 (same as SEQ ID NOs: 53, 57 and 73) |
| | | DIQMTQSPSSLSASVGDRVTITCKASQDVSIGVAWY |
| | | QQKPGKAPKLLIYSASYRYTGVPSRFSGSGSGTDFT |
| | | LTISSLQPEDFATYYCQQYYIYPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 6A23 | Chain 1 | SEQ ID NO: 74 |
| | | DIQMTQSPSSLSASVGDRVTITCKASQDVSIGVAWY |
| | | QDKPGKAPKLLIYSASYRYTGVPSRFSGSGSGTDFT |
| | | LTISSLQPEDFATYYCQQYYIYPYTFGCGTKVEIKR |
| | | TVAAQVQLVQSGAEVKKPGESLKVSCKASGYTFTS |
| | | YYMHWVRQAPGQGLEWMGIINPSGGSTSYAQKFQ |
| | | GRVTMTRDTSTSTVYMELSSLRSEDTAVYYCARGS |
| | | AYYYDFADYWGQGTLVTVSS |
| | Chain 2 | SEQ ID NO: 75 |
| | | SYVLTQPSSVSVAPGQTATISCGGHNIGSKNVHWY |
| | | QQRPGQSPVLVIYQDNKRPSGIPERFSGSNSGNTAT |
| | | LTISGTQAMDEADYYCQVWDNYSVLFGGGTKLTV |
| | | LRTVAAEVQLVESGGGLVQPGGSLRLSCAASGFTF |
| | | TDYTMDWVRKAPGKCLEWVADVNPNSGGSIYNQ |
| | | RFKGRFTLSVDRSKNTLYLQMNSLRAEDTAVYYC |
| | | ARNLGPSFYFDYWGQGTLVTVSSASEPKSSDKTHT |
| | | CPPCPAPPVAGPSVFLFPPKPKDTLMISRTPEVTCVV |
| | | VDVSQEDPEVKFNWYVDGVEVHNAKTKPREEQFN |
| | | STYRVVSVLTVLHQDWLNGKEYKCKVSNKGLPSSI |
| | | EKTISKAKGQPREPQVYTLPPSRDELTKNQVSLWC |
| | | LVKGFYPSDIAVEWESNGQPENNYKTTPPVLDSDG |
| | | SFFLYSKLTVDKSRWQQGNVFSCSVMHEALHNHY |
| | | TQKSLSLSP |
| | Chain 3 | SEQ ID NO: 64 (same as SEQ ID NOs: 76, 80 and 84) |
| | | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPPVAGPSVFLF |
| | | PPKPKDTLMISRTPEVTCVVVDVSQEDPEVKFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSRDELTKNQVSLSCAVKGFYPSDIAVEWES |
| | | NGQPENNYKTTPPVLDSDGSFFLVSKLTVDKSRWQ |
| | | QGNVFSCSVMHEALHNRFTQKSLSLSP |
| | Chain 4 | SEQ ID NO: 65 (same as SEQ ID NOs: 77, 85 and 89) |
| | | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQHYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 6A25 | Chain 1 | SEQ ID NO: 78 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQAYTTPPTFGCGTKVEIK |
| | | RTVAAEVQLVESGGGLVQPGGSLRLSCAASGFTFS |
| | | TYAMNWVRDAPGKGLEWVGRIRSKYNNYATYYA |
| | | DSVKGRFTISRDDSKNSLYLQMNSLRAEDTAVYYC |
| | | VRHGNFGNSYVSWFAYWGQGTLVTVSS |
| | Chain 2 | SEQ ID NO: 47 (same as SEQ ID NOs: 79 and 83) |
| | | QAVVTQEPSLTVSPGGTVTLTCRSSTGAVTTSNYA |
| | | NWVQKKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFNIKDTYIHWVRKAPGKCLEWVARIYPTNGYT |
| | | RYADSVKGRFTISADTSKNTAYLQMNSLRAEDTAV |
| | | YYCSRWGGDGFYAMDYWGQGTLVTVSSASEPKSS |
| | | DKTHTCPPCPAPPVAGPSVFLFPPKPKDTLMISRTPE |
| | | VTCVVVDVSQEDPEVKFNWYVDGVEVHNAKTKP |
| | | REEQFNSTYRVVSVLTVLHQDWLNGKEYKCKVSN |
| | | KGLPSSIEKTISKAKGQPREPQVYTLPPSRDELTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSP |
| | Chain 3 | SEQ ID NO: 64 (same as SEQ ID NOs: 76, 80 and 84) |
| | | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPPVAGPSVFLF |
| | | PPKPKDTLMISRTPEVTCVVVDVSQEDPEVKFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSRDELTKNQVSLSCAVKGFYPSDIAVEWES |
| | | NGQPENNYKTTPPVLDSDGSFFLVSKLTVDKSRWQ |
| | | QGNVFSCSVMHEALHNRFTQKSLSLSP |
| | Chain 4 | SEQ ID NO: 81 |
| | | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQAYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 6A26 | Chain 1 | SEQ ID NO: 82 |
| | | DIQMTQSPSSLSASVGDRVTITCRASQDVNTAVAW |
| | | YQDKPGKAPKLLIYSASFLYSGVPSRFSGSRSGTDF |
| | | TLTISSLQPEDFATYYCQQHYTTPPTFGCGTKVEIK |
| | | RTVAAEVQLVESGGGLVQPGGSLRLSCAASGFTFS |
| | | TYAMNWVRDAPGKGLEWVGRIRSKYNNYATYYA |
| | | DSVKGRFTISRDDSKNSLYLQMNSLRAEDTAVYYC |
| | | VRHGNFGNSYVSWFAYWGQGTLVTVSS |
| | Chain 2 | SEQ ID NO: 47 (same as SEQ ID NOs: 79 and 83) |
| | | QAVVTQEPSLTVSPGGTVTLTCRSSTGAVTTSNYA |
| | | NWVQKKPGQAPRGLIGGTNKRAPWTPARFSGSLL |
| | | GGKAALTLSGAQAEDEADYYCALWYSNLWVFGG |
| | | GTKLTVLRTVAAEVQLVESGGGLVQPGGSLRLSCA |
| | | ASGFNIKDTYIHWVRKAPGKCLEWVARIYPTNGYT |
| | | RYADSVKGRFTISADTSKNTAYLQMNSLRAEDTAV |
| | | YYCSRWGGDGFYAMDYWGQGTLVTVSSASEPKSS |
| | | DKTHTCPPCPAPPVAGPSVFLFPPKPKDTLMISRTPE |
| | | VTCVVVDVSQEDPEVKFNWYVDGVEVHNAKTKP |
| | | REEQFNSTYRVVSVLTVLHQDWLNGKEYKCKVSN |
| | | KGLPSSIEKTISKAKGQPREPQVYTLPPSRDELTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSP |
| | Chain 3 | SEQ ID NO: 64 (same as SEQ ID NOs: 80, 84 and 84) |
| | | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPPVAGPSVFLF |
| | | PPKPKDTLMISRTPEVTCVVVDVSQEDPEVKFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSRDELTKNQVSLSCAVKGFYPSDIAVEWES |
| | | NGQPENNYKTTPPVLDSDGSFFLVSKLTVDKSRWQ |
| | | QGNVFSCSVMHEALHNRFTQKSLSLSP |
| | Chain 4 | SEQ ID NO: 65 (same as SEQ ID NOs: 77, 85 and 89) |
| | | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQHYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |

### D. Antibody Variants

The engineered antibodies provided herein also encompass various variants of the antibody sequences provided herein.

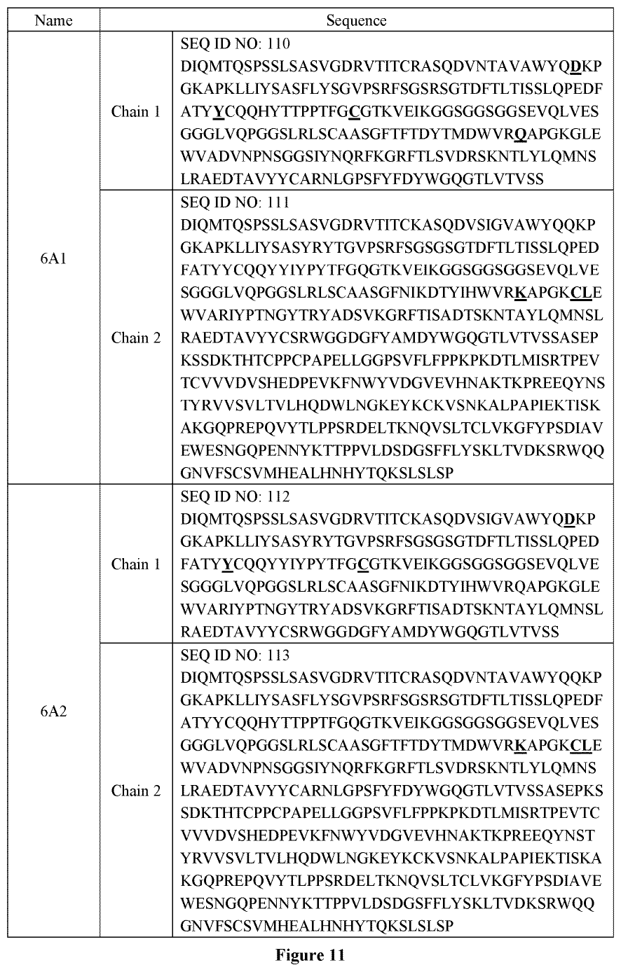

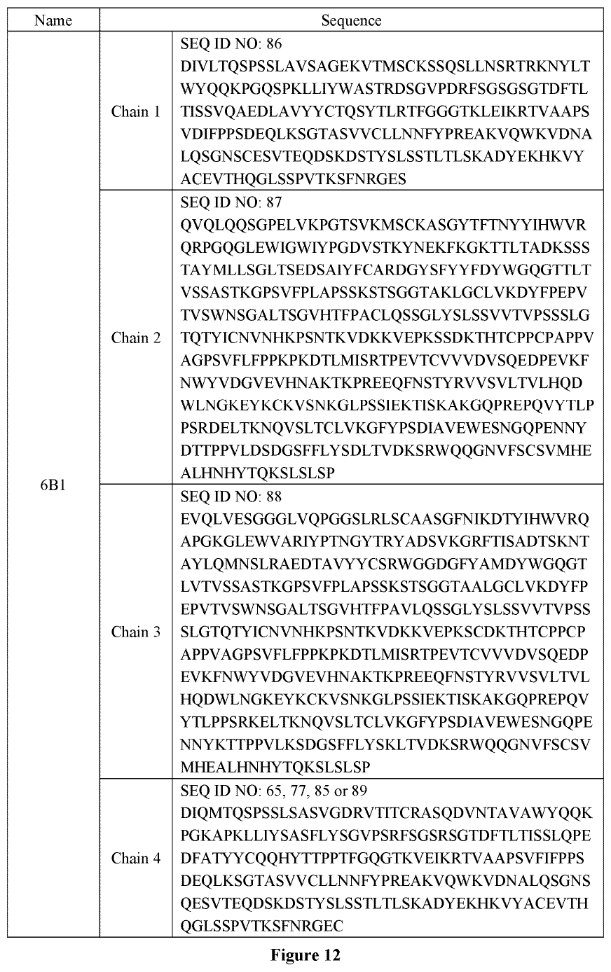

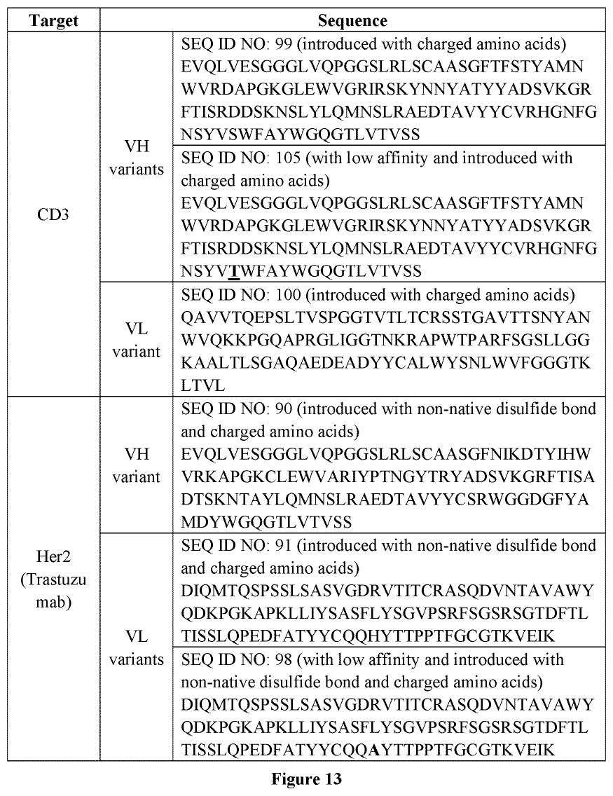

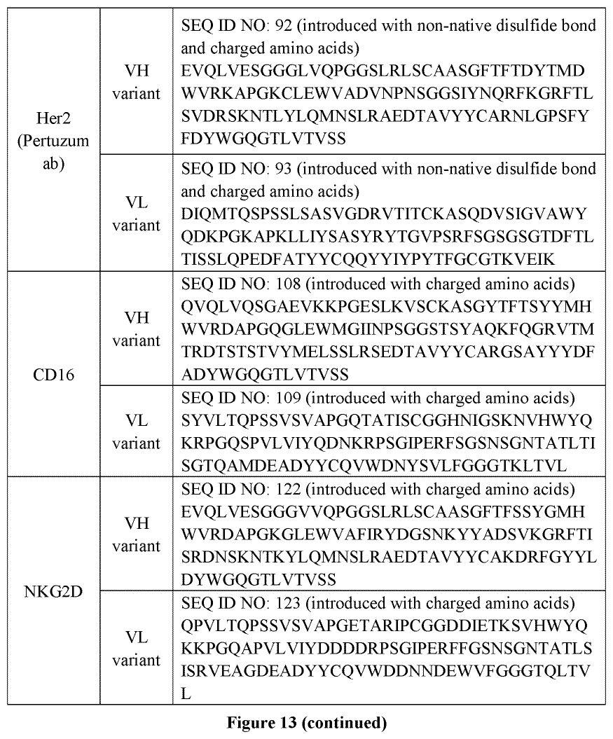

In certain embodiments, the antibody variants comprise one or more modifications or substitutions in one or more of the CDR regions, non-CDR regions and/or constant regions (e.g., Fc regions) as provided in the FIG. 10 above. Such variants retain binding specificity to the corresponding targets of their parent antibodies but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g., one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions in one or more CDR regions or FR regions in the heavy or light chain variable region sequences as provided in Tables 1 to 4 above. FR sequences can be readily identified by a person skilled in the art based on the CDR sequences and variable region sequences in Tables 1 to 4 above, as it is well-known in the art that a CDR region is flanked by two FR regions in the variable region. The affinity variants retain specific binding affinity to the targets of the parent antibody, or even have improved target specific binding affinity over the parent antibody. In certain embodiments, at least one (or all) of the substitution(s) in the CDR sequences, FR sequences, or variable region sequences comprises a conservative substitution.

A person skilled in the art will understand that in the CDR regions and variable region sequences provided in Tables 1 to 4 above, one or more amino acid residues may be substituted yet the resulting antibody or antigen-binding fragment still retain the binding affinity or binding capacity to the targets, or even have an improved binding affinity or capacity. Various methods known in the art can be used to achieve this purpose. For example, a library of antibody variants (such as Fab or scFv variants) can be generated and expressed with phage display technology, and then screened for the binding affinity to the targets. For another example, computer software can be used to virtually simulate the binding of the antibodies to the targets and identify the amino acid residues on the antibodies which form the binding interface. Such residues may be either avoided in the substitution so as to prevent reduction in binding affinity or targeted for substitution to provide for a stronger binding.

In certain embodiments, the engineered antibodies comprise one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitution(s) in the CDR sequences and/or FR sequences in total.

In certain embodiments, the engineered antibodies comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Tables 1 to 4 above yet retaining the specific binding affinity to the corresponding targets at a level similar to or even higher than its parent antibody.

In certain embodiments, the engineered antibodies comprise one or more variable region sequences having at least 80% (e.g., at least 85%, 88%, 90%, 91%, 92%, 93%, 94%, 95%, 96%, 97%, 98%, 99%) sequence identity to that (or those) listed in Tables 1 to 4 above yet retaining the specific binding affinity to the corresponding targets at a level similar to or even higher than its parent antibody. In some embodiments, a total of 1 to 10 amino acids have been substituted, inserted, or deleted in a variable region sequence listed in Tables 1 to 4 above. In some embodiments, the substitutions, insertions, or deletions occur in regions outside the CDRs (e.g., in the FRs).

### Glycosylation Variants

The engineered antibodies provided herein also encompass glycosylation variants, which can be obtained to either increase or decrease the extent of glycosylation of the antibodies.

The engineered antibodies may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the engineered antibodies provided herein comprise a mutation at N297 (e.g., N297A, N297Q, or N297G) to remove the glycosylation site.

### E. Conjugates

The engineered antibodies as provided herein can be used in a non-conjugated form or in a conjugated form.

In a conjugated form, the engineered antibodies are conjugated to one or more desired conjugate moieties, i.e. heterologous moieties, to realize certain functionalities, e.g. to facilitate target detection or for imaging or therapy.

Herein, the present disclosure provides a conjugate, which comprises the engineered antibody provided herein, and a conjugate moiety (e.g. payload) that is conjugated thereto. The payload can be any one of the group consisting of a radioactive label, a fluorescent label, an enzyme-substrate label, an affinity purification tag, a tracer molecule, an anticancer drug, and a cytotoxic molecule.

A variety of conjugates can be linked to the engineered antibody provided herein by covalent binding, affinity binding, intercalation, coordinate binding, complexation, association, blending, or addition, among others. (see, e.g., “Conjugate Vaccines”, Contributions to Microbiology and Immunology, J. M. Cruse and R. E. Lewis, Jr. (eds.), Carger Press, New York, (1989)).

In certain embodiments, the engineered antibody provided herein may be engineered to contain specific sites outside the epitope binding portion that may be specifically utilized for binding to one or more conjugates. For example, such a site may include one or more reactive amino acid residues, such as for example cysteine or histidine residues, to facilitate covalent linkage to a conjugate.

In certain embodiments, the N-terminus and/or C-terminus of the engineered antibody provided herein can also serve to provide reactive groups for conjugation. For example, the N-terminus can be conjugated to one moiety (e.g. polyethylene glycol (PEG), etc.) and the C-terminus is conjugated to another moiety (e.g. biotin, etc.).

In certain embodiments, the engineered antibody provided herein may be linked to a conjugate directly, or indirectly for example through another conjugate or through a linker.

For example, the engineered antibody provided herein having a reactive residue such as cysteine may be linked to a thiol-reactive agent in which the reactive group is, for example, a maleimide, an iodoacetamide, a pyridyl disulfide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

For another example, the engineered antibody provided herein may be conjugated to biotin, then indirectly conjugated to a second conjugate that is conjugated to avidin. For still another example, the engineered antibody may be linked to a linker which further links to the conjugate. Examples of linkers include bifunctional coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suherate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and his-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio) pentanoate (SPP) to provide for a disulfide linkage.

In certain embodiments, the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

The conjugate moiety can be a detectable label, a pharmacokinetic modifying moiety, a purification moiety, a cytotoxic moiety or a therapeutic agent. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection.

In certain embodiments, the conjugate moiety can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. The polymer may be of any molecular weight and may be branched or unbranched. The number of polymers attached to the antibody may vary, and if more than one polymer is attached, they can be the same or different molecules. In certain embodiments, the conjugate can be a purification moiety such as a magnetic bead.

In certain embodiments, the conjugate moiety can be a cytotoxic moiety. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In some embodiments, the conjugate moiety comprises an enzymatically active toxin or a fragment thereof, including but not limited to diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes.

The therapeutic agents or drugs useful as the conjugate moiety can be those which are useful for treating a disease or disorder related to HER2.

Methods for the conjugation of conjugate moieties to proteins such as antibodies, immunoglobulins or fragments thereof are found, for example, in U.S. Pat. No. 5,208,020; U.S. Pat. No. 6,4411,163; WO2005037992; WO2005081711; and WO2006/034488, which are incorporated herein by reference to the entirety.

In certain embodiments, the engineered antibodies provided herein are used as a base for a conjugate.

### F. Polynucleotides and Recombinant Methods

The present disclosure provides isolated polynucleotides that encode the engineered antibodies provided herein. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The polynucleotides encoding the engineered antibodies disclosed herein may be generated using methods known in the art. In certain embodiments, the sequence of the polynucleotides may be obtained based on the amino acid sequences of the engineered antibodies, and nucleic acids can be generated using synthetic methods. Alternatively, the polynucleotides provided herein can also be obtained from another available nucleic acid that encodes a polypeptide with a sequence homologous to the polypeptides in the engineered antibodies disclosed herein. Then a DNA manipulation process can be applied to manipulate the sequence of the parent antibody-encoding nucleic acid, such as introducing mutations, insertion, deletion, etc., so as to obtain the nucleic acid encoding the engineered antibodies disclosed herein.

The isolated polynucleotide that encodes the engineered antibodies can be inserted into one or more vector(s) for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1a), a transcription termination sequence, and one or more other regulatory elements.

The present disclosure provides vectors comprising the isolated polynucleotides provided herein. In certain embodiments, the polynucleotides provided herein encodes the engineered antibodies, with at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the engineered antibodies can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for the engineered antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for antibody production and cultured in conventional nutrient media modified as appropriate for inducing promoters, selecting transformants, or amplifying the genes encoding the desired sequences. In another embodiment, the antibodies may be produced by homologous recombination known in the art. In certain embodiments, the host cell is capable of producing the antibodies provided herein.

The present disclosure also provides a method of expressing the antibodies provided herein, comprising culturing the host cell provided herein under the condition at which the vector of the present disclosure is expressed. The host cells used to produce the antibodies provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibodies can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The engineered antibodies prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### III. Pharmaceutical Formulations and Administration

The present disclosure also provides a pharmaceutical composition. Besides the polypeptide complex as described above, the pharmaceutical composition further comprises a pharmaceutically acceptable carrier.

As used herein, the term “pharmaceutically acceptable” indicates that the designated carrier, vehicle, diluent, excipient(s), salt and/or medium is generally chemically and/or physiologically compatible with other ingredients, such as the active ingredient (i.e. the polypeptide complex or the heterodimeric antibody or antigen-binding fragment thereof) comprising the formulation, and is physiologically compatible with a subject receiving the pharmaceutical composition.

A “pharmaceutically acceptable carrier” refers to an ingredient in a pharmaceutical formulation, other than an active ingredient, which is bioactivity acceptable and nontoxic to a subject. In the context of the present disclosure, a pharmaceutical acceptable carrier for use in the pharmaceutical composition disclosed herein may include, for example, pharmaceutically acceptable liquid, gel or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Herein, suitable “components” may include, for example, antioxidants, fillers, binders, disintegrants, buffers, preservatives, lubricants, flavorings, thickeners, coloring agents, emulsifiers or stabilizers such as sugars and cyclodextrins. Suitable “antioxidants” may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a pharmaceutical composition provided herein decreases oxidation of the polypeptide complex or heterodimeric antibody or antigen-binding fragment thereof. This reduction in oxidation prevents or reduces loss of binding affinity, thereby improving protein stability and maximizing shelf-life. Therefore, in certain embodiments, a pharmaceutical composition is provided that comprise, in addition to the active ingredient (i.e. the polypeptide complex or the heterodimeric antibody or antigen-binding fragment thereof disclosed herein), one or more antioxidants such as methionine.

The pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Pharmaceutically acceptable “diluents” may include saline and aqueous buffer solutions.

Pharmaceutically acceptable “adjuvants” may include preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving the polypeptide complex as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the polypeptide complex, the polypeptide complex. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

In certain embodiments, a composition is further provided, comprising a pharmaceutically acceptable carrier, diluent or adjuvant, and an active ingredient. The active ingredient can be the engineered antibody or antigen-binding fragment thereof disclosed herein, or the engineered antibody conjugate disclosed herein.

### IV. Kits

In another aspect, the present invention provides kits containing the engineered antibody provided herein and directions for using the engineered antibody. The kit may also include a container and optionally one or more vial, test tube, flask, bottle, or syringe. Other formats for kits will be apparent to those of skill in the art and are within the scope of the present invention.

### V. Medical Use

In another aspect, the present invention provides a method for treating, preventing or alleviating a disease condition in a subject that is in need of such treatment, comprising: administering to the subject a therapeutically effective amount of the engineered antibody of the present disclosed herein, or the polynucleotide encoding the engineered antibody provided herein, or the pharmaceutical composition provided herein.

As used herein, the term “subject” or “individual” or “animal” or “patient” refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on. In certain embodiments, the subject is human.

As used herein, “treatment” of a condition may include, alleviating a condition, slowing the onset or rate of development of a condition, delaying the development of symptoms associated with a condition, reducing or ending symptoms associated with a condition, generating a complete or partial regression of a condition, curing a condition, or some combinations thereof.

As used herein, the term “disorder,” “disease,” “condition” or alike, refers to a condition that affects a subject who would nonetheless benefits from treatment with the engineered antibody.

In certain embodiments, the disease or disorder is a HER2-related disease or disorder. In some embodiment, the HER2-related condition or a disorder is cancer.

By “cancer” herein is meant any medical condition characterized by malignant cell growth or neoplasm, abnormal proliferation, or infiltration or metastasis. Cancer includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid.

In some embodiment, the HER2-related condition or a disorder is a HER2-expressing cancer, a HER2-overexpressing cancer, or a HER ligand overexpressing cancer.

A “HER2-expressing cancer” is one that involves cancer cells or tumor cells having HER2 protein present at their cell surface. In HER2-expressing cancer cells, homodimers formed by HER2 or heterodimers formed by HER2 along with HER1, HER3 or HER4 can activate the PI3K/Akt and MAPK pathways in cells, thus regulating the proliferation, differentiation, migration and apoptosis of tumor cells.

A “HER2-overexpressing cancer” is one which has significantly higher levels of a HER receptor, such as HER2, at the cell surface of a cancer or tumor cell, compared to a noncancerous cell of the same tissue type. Such overexpression may be caused by gene amplification or by increased transcription or translation.

HER2 expression or overexpression may be determined in a diagnostic or prognostic assay by evaluating increased levels of the HER2 protein present on the surface of a cell (e.g. via an immunohistochemistry assay; IHC). Alternatively, or additionally, one may measure levels of HER2-encoding nucleic acid in the cell, e.g. via fluorescent in situ hybridization (FISH; see WO98/45479 published October 1998), southern blotting, or polymerase chain reaction (PCR) techniques, such as real time quantitative PCR (RT-PCR). One may also study HER2 receptor overexpression by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132:73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody. In some embodiments, the test sample is derived from a cancer cell or tissue.

As used herein, the term “therapeutically effective amount” of a therapeutic agent refers to an amount of the therapeutic agent that, when taken by a subject in an appropriate manner, can generate sufficient therapeutic effects to the subject. It is to be understood that just like other therapeutic drugs, the therapeutically effective amount of the polypeptide complex as provided above will be influenced by various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the engineered antibody or antigen-binding fragment as provided herein may be administered at a therapeutically effective amount of about 0.01 mg/kg to about 100 mg/kg. Dosage regimens may be adjusted to provide the optimum desired response (e.g., a therapeutic response). For example, a single dose may be administered, or several divided doses may be administered over time.

The engineered antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the engineered antibodies or antigen-binding fragments disclosed herein may be administered alone or in combination with one or more additional therapeutic means or agents. For example, the engineered antibodies or antigen-binding fragments disclosed herein may be administered in combination with another therapeutic agent, for example, a chemotherapeutic agent or an anti-cancer drug.

In certain of these embodiments, the engineered antibodies or antigen-binding fragment as disclosed herein that is administered in combination with one or more additional therapeutic agents may be administered simultaneously with the one or more additional therapeutic agents, and in certain of these embodiments the engineered antibodies or antigen-binding fragment and the additional therapeutic agent(s) may be administered as part of the same pharmaceutical composition. However, the engineered antibodies or antigen-binding fragments administered “in combination” with another therapeutic agent does not have to be administered simultaneously with or in the same composition as the agent. The engineered antibodies or antigen-binding fragments administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Construction, Expression, and Purification of Antibodies

### 1.1 Structure of Each Antibody

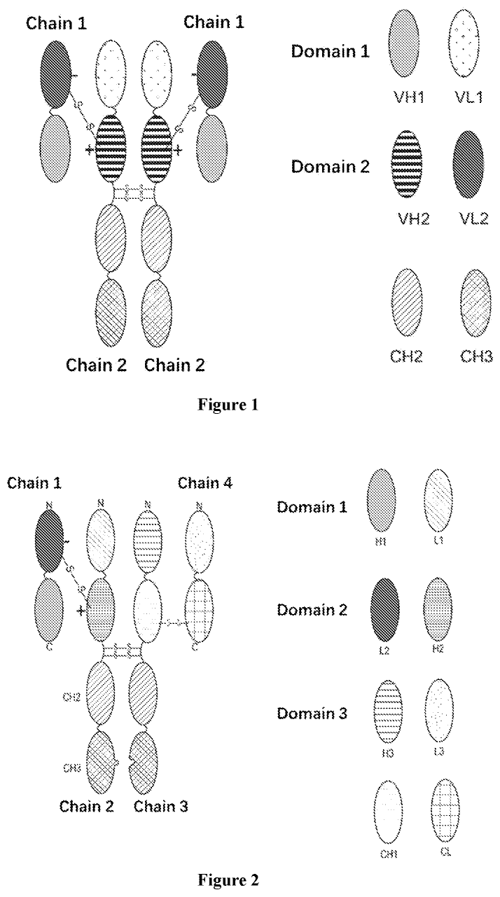

According to the structures of DICAD (see patent application: CN201711415979.9) and TRIAD (see patent application: CN201880081812.X), a bispecific antibody with the structure shown in FIG. 1 and a trispecific antibody with the structure shown in FIG. 2 were constructed.

In the bispecific antibody DICAD, two domains targeting antigens are included, domain 1 (associated by VH1 and VL1) and domain 2 (associated by VH2 and VL2). In the trispecific antibody TRIAD, three domains targeting antigens are included, domain 1 (associated by VH1 and VL1), domain 2 (associated by VH2 and VL2) and domain 3 (associated by VH3 and VL3).

In the DICAD structure, the structures of each peptide chain from N-terminal to C-terminal are shown below:
Chain 1: VL1-linker-VH2-hinge region-CH2-CH3
Chain 2: VL2-linker-VH1

In the TRIAD structure, the structures of each peptide chain from N-terminal to C-terminal are shown below:
Chain 1: VL2-linker-VH1
Chain 2: VL1-linker-VH2-hinge region-CH2-CH3
Chain 3: VH3-CH1-hinge region-CH2-CH3
Chain 4: VL3-CL

DICAD antibodies (6A1 and 6A2) and TRIAD antibodies (6A8, 6A12, 6A14, 6A15, 6A17, 6A19, 6A17, 6A18, 6A21, 6A23, 6A25 and 6A26) were constructed based on the structure of each aforementioned polypeptide chain. The antigen bound by each antigen-targeting domain of each antibody are shown in Table 9 below, wherein Tra stands for Trastuzumab and Per stands for Pertuzumab.

TABLE 9
Domain order of each peptide chain of the antibodies

Structure

| Antibody | Domain 1 | Domain 2 | Domain 3 |
|---|---|---|---|
| 6A1 | Her2 (Per) | Her2 (Tra) | |
| 6A2 | Her2 (Tra) | Her2 (Per) | |
| 6A8 | CD3(low | Her2 (Tra) | Her2 (Per) |
| | affinity) | | |
| 6A12 | NKG2D | Her2 (Tra) | Her2 (Per) |
| 6A14 | CD3 | Her2 (Tra) | Her2 (Per) |
| 6A15 | NKG2D | Her2 (Tra) | Her2 (Per) |
| 6A17 | NKG2D | Her2 (Tra) | Her2 (Per) |
| 6A19 | CD3 | Her2 (Per) | Her2 (Tra) |
| 6A18 | CD16 | Her2 (Tra) | Her2 (Per) |
| 6A21 | Her2 (Tra) | CD3 | Her2 (Per) |
| 6A23 | CD16 | Her2 (Per) | Her2 (Tra) |
| 6A25 | CD3 | Her2 (Tra, | Her2 (Tra, |
| | | low affinity) | Low affinity) |
| 6A26 | CD3 | Her2 (Tra) | Her2 (Tra) |
| 6B1 | | | |

TABLE 10
Domains of 6A1, 6A2

| | | Domain Order | | |
|---|---|---|---|---|
| | Polypeptide | from N to C | Mutation List | |
| Antibody | Chain | and Sequence | Domain 1 | Domain 2 |
| 6A1 | Chain 1 | VL2 (HER2-Tra)- | / | Q38D, |
| | (SEQ ID | linker-VH1(HER2- | | Q100C |
| | NO: 110) | Per) | | in VL2 |
| | Chain 2 | VL1(HER2-Pcr)- | / | Q39K, |
| | (SEQ ID | linker-VH2(HER2- | | G44C |
| | NO: 111) | Tra)-hinge-CH2- | | in VH2 |
| | | CH3 | | |
| 6A2 | Chain 1 | VL2 (HER2-Per)- | / | Q38D, |
| | (SEQ ID | linker-VH1(HER2- | | Q100C |
| | NO: 112) | Tra) | | in VL2 |
| | Chain 2 | VL1(HER2-Tra)- | / | Q39K, |
| | (SEQ ID | linker-VH2(HER2- | | G44C |
| | NO: 113) | Per)-hinge-CH2- | | in VH2 |
| | | CH3 | | |

Notes:
“/”: No mutations.

The Fv sequences that used in the construction of the DICAD and TRIAD antibodies, and that bind each antigenic target (Her2, CD3, NKG2D, and CD16) are shown in Tables 1 to 4, respectively.

In the construction of the DICAD and TRIAD antibodies, point mutations are introduced at the designated points (Kabat) of the VH and VL structural domains, as listed in Tables 5-7, and 10. Specifically, each antibody has the structure and mutation shown in Tables 5-7, wherein the mutation in the CH3 region corresponds to the mutation in human IgG. Full-length sequences and VH/VL sequences used in each of TRIAD antibodies are also provided in Tables 5-7. Full-length sequences used in each of DICAD antibodies are provided in Table 10.

6A1 comprises a Chain 1 sequence as shown in SEQ ID NO: 110 (i.e., VL2 (HER2-Tra)-linker-VH1 (HER2-Per)) and a Chain 2 sequence as shown in SEQ ID NO: 111 (i.e., VL1 (HER2-Per)-linker-VH2 (HER2-Tra)-hinge-CH2-CH3). 6A2 comprises a Chain 1 sequence as shown in SEQ ID NO: 112 and a Chain 2 sequence as shown in SEQ ID NO: 113. 6A1 and 6A2 are otherwise identical except that 6A2 comprises point mutations Q39D in the VH1 (HER2-Per) and Q38K in the VL1 (HER2-Per) to introduce electrostatic interaction.

6B1 comprises a Chain 1 sequence as shown in SEQ ID NO: 86, a Chain 2 sequence as shown in SEQ ID NO: 87, a Chain 3 sequence as shown in SEQ ID NO: 88 and a Chain 4 sequence as shown in SEQ ID NO: 89. 6B1 was used as a positive control.

### 1.2 Gene Synthesis, Expression, and Purification of Antibodies

All amino acid sequences of the bispecific antibodies are provided in Table 8 of the present disclosure. Polynucleotides encoding these bispecific antibodies are designed, and codon optimized using OptimumGene before proceeding to synthesis.

The target gene was first constructed in the pUC57 vector and subsequently subcloned into the pTGE5 vector. DNA was prepared by Maxiprep for transfection.

CHO3E7 cells were cultured and passaged at a concentration of 0.3×10⁶ cells/ml. Transfection was conducted when the cell density reached 1.8-2.5×10⁶ cells/ml. First, 300 μl DNA heavy and light strands were added to 50 ml Freestyle CHO medium and mixed with gentle shaking. Subsequently, 3 mg PEI transfection reagent was added and mixed with gentle shaking for more than 3 minutes. The mixture was allowed to stand at 37° C. for 7 min and subsequently added to 450 ml cell suspension to obtain a total volume of 500 ml. 25 ml TN1 (master mix concentration 200 g/L) was added to the mixture after 24 hours.

1 ml suspension was collected on day 1, day 3 and day 5 after transfection for testing. A 50 μl sample was collected for cell counting, and the remaining sample was processed by centrifugation at 3000 rpm for 5 min, after which the supernatant was left at −20° C. On day 6, the cultures were harvested and processed by centrifugation at 5500 rpm for 30 min. The supernatant was separated and filtered through a 0.22 μm filter, and the protein was further purified.

Chromatography column: 5 ml Monofinity A resin (GenScript, Batch No. L00433) chromatography column; equilibration buffer A: 20 mM PB, 150 mM NaCl, pH 7.2; Washing buffer B: 50 mM citric acid, pH 3.5; Neutralization buffer C: 1 M Tris-HCl, pH 9.0; Flow rate: 2 ml/min; Gradient: 100% gradient elution. After separation, 0.155 ml of neutralization buffer C was added to each 1 ml fraction. The collected protein solution was dialyzed in PBS (pH 7.2) at 4° C. for 16 h.

The aforementioned samples were analyzed by SDS-PAGE followed by Western blot analysis. The samples were purified by SEC and analyzed for purity.

### Example 2. Detection of Cell Growth Inhibition of Cells with High Her2 Expression Level by DICAD Antibody

In order to compare the effect of the constructed bispecific antibodies on cells with high Her2 expression level, 6A1, 6A2, trastuzumab (Herceptin), and pertuzumab (Perjeta) were added to BT-474 cells to observe the inhibitory effect on the growth of BT-474 cells.

The SRB method was applied to detect the inhibitory effect of drugs on the proliferation and growth of tumor cells. The main procedures are as follows

Cells of logarithmic growth phase were seeded in 96-well culture plates, and different concentrations (3, 10, 30, 100, 300, 1000, 3000, 10000 ng/ml) of the drug were added, with 2 replicate wells for each concentration and a control for the corresponding concentration of solvent. The tumor cells were cultured at 37° C. and 5% CO2 for 120 hours. Cells were stained with SRB at room temperature and finally lysed by adding Tris solution. The OD values were measured by a microplate reader (BioTek) at 510 nm wavelength. The cell growth inhibition rate was calculated by using the following equation:

$$Inhibition rate = \text{} \\ ( OD values of control wells - OD values of wells with drugs ) \text{} \text{} / OD values of control wells \times 100 \%$$

Based on the inhibition rate of each concentration, the half-inhibitory concentration IC50 was calculated based on the nonlinear regression method.

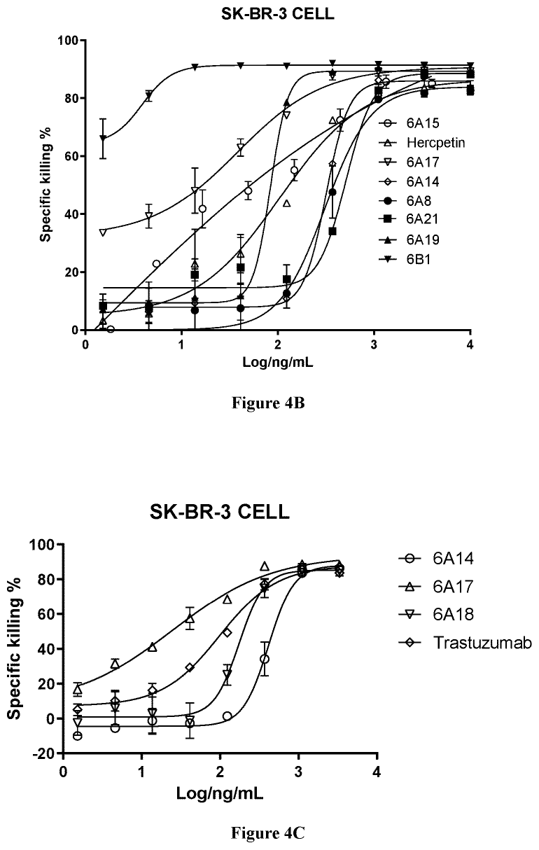

The results are shown in FIG. 3. The results showed that trastuzumab monoclonal antibody alone and in combination with pertuzumab monoclonal antibody had a blocking effect. In contrast, the bispecific antibodies 6A1 and 6A2 constructed with DICAD using the aforementioned monoclonal antibodies VH and VL did not show a blocking effect, but unexpectedly showed a significant agonistic effect.

The above results exhibited by 6A1 and 6A2 suggest that the 2:2 antibodies constructed using the DICAD model are not effective. The inventor postulate that the possible reason for this is that the antigen-binding domains corresponding to trastuzumab and pertuzumab, which bind Her2 to ECD2 and ECD4 respectively, are too close together in the antibodies constructed through the DICAD platform, which brings Her2 monomers closer to each other and enhances Her2 dimer formation, thereby promoting her2-dependent tumor growth. Therefore, this construct reverses the Her2 signaling pathway blocking effect of parent antibodies (trastuzumab and pertuzumab), and instead exhibits agonistic manifestation.

### Example 3. Performance Testing of TRIAD Antibodies

3.1 Killing Assay of Cells with High Her2 Expression Level

In order to test the ability of antibodies to kill cells with high Her2 expression level, SK-BR-3 cell killing assay was conducted.

The assay medium used was RPMI medium 1640 supplemented with 2% HI-FBS. The assay plate used was Corning 96-well white flat-bottom microplate (Cat.No. 3903). SK-BR-3 cells, provided by ATCC, were cells with high Her2 expression level used for testing.

### Method

1) SK-BR-3 cells were re-suspended at a density of 3E5 cells/mL with RPMI1640+2% FBS. Dispense 100 μL to each well of plate (3903). Incubate at 37° C. overnight.
2) Prepare PBMC cells, re-suspended at a density of 3E5 cells/mL with RPMI1640+2% FBS. Add 100 μL to SK-BR-3 plate.
3) Prepared the antibody at a concentration of 180 μg/mL with PBS (9 doses, 3-fold diluted, duplicate). Add 10 μL to plate.
4) Incubate the plate in incubator for 72h.
5) Remove the supernatant from the plate, wash twice with 100 μL PBS, then add 100 μL 1640+2% FBS. Detect the CTG signal.

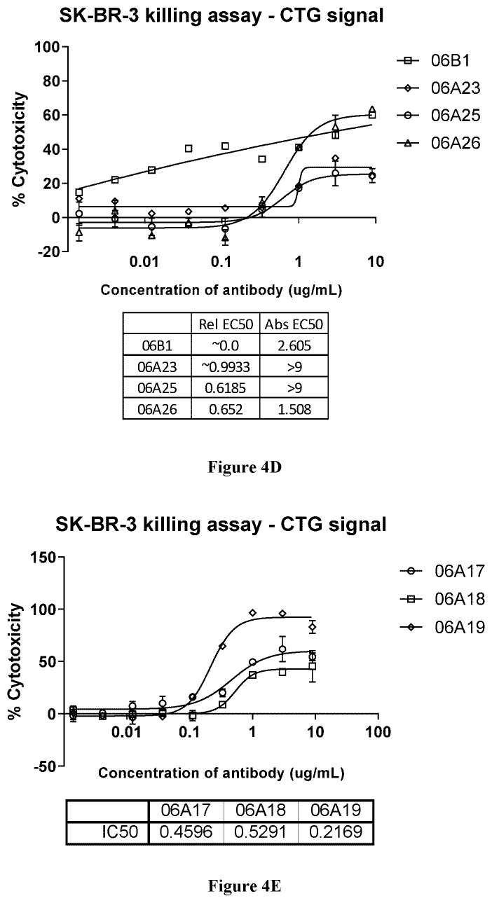

The results are shown in FIGS. 4A-4F, which indicated that 6B1 has the highest killing activity. 6A19 and 6A26 arc slightly weaker, and 6A17, 6A25, 6A23 and 6A15 are slightly stronger than trastuzumab. Compared with trastuzumab, antibodies using the above construction method show enhanced PBMC dependent antibody killing effect.

3.2 Results of the Cell Line with Low Her2 Expression Level
MCF-7 killing assay study design
Assay medium: RPMI medium 1640 supplemented with 2% HI-FBS
Assay plate: Corning 96-well white flat-bottom microplate (Cat.No. 3903)
MCF-7 cell: ATCC HTB-22
Culture medium: EMEM+0.01 mg/mL Insulin+10% FBS+1% P.S.

TABLE 11
Materials used in the MCF-7 killing assay

| | Vendor | Cat. No |
|---|---|---|
| RPMI-1640 | Gibco | 11875119 |
| FBS | Biological | 04-002-1A |
| | Industries | |
| CellTiter-Glo | Promega | G7573 |

### Methods

1) Prepare MCF-7 cells, re-suspended at a density of 1.5E5 cells/ml with RPMI1640+2% FBS. Dispense 100 μL to each well of plate (3903). Incubate at 37° C. overnight.
2) Prepare PBMC cells, re-suspended at a density of 7.5E5 cells/ml with RPMI1640+2% FBS. Add 100 μL to MCF-7 plate.
3) Prepared the antibody at a concentration of 540 μg/mL with PBS (9 doses, 3-fold diluted). Add 10 μL to plate.
4) Incubate the plate in incubator for 72h.
5) Remove the supernatant from the plate, wash twice with 100 μL PBS, then add 100 μL 1640+2% FBS. Detect the CTG signal.

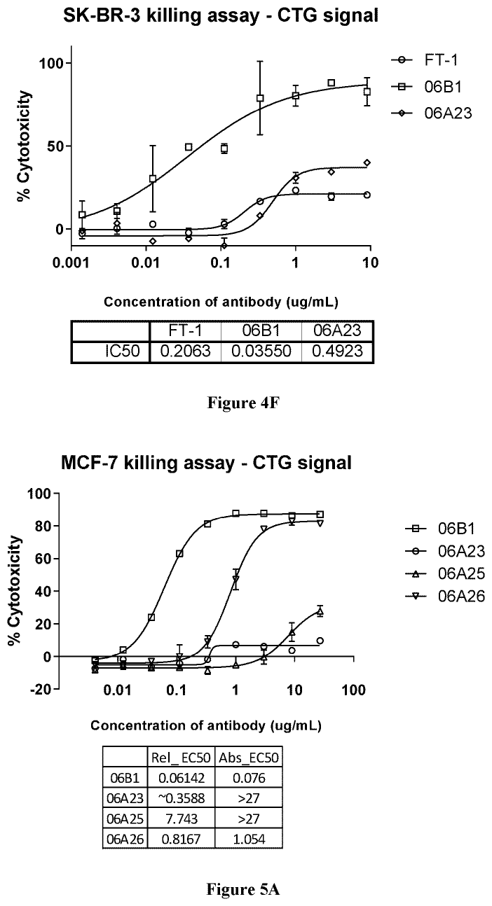

The results in FIG. 5A and FIG. 5B showed that 6B1 had the highest killing activity, followed by 6A19 and 6A26, and then followed by 6A25. 6A23, 6A17, 6A18 and trastuzumab show no obvious killing effect.

3.3 In Vivo Results of Cell Line with High Her2 Expression Level
3.3.1 Animal Model of KPL-4 with High Her2 Expression Level

### Experimental Protocols and Methods

Six-week-old female B-NDG mice were used, and each mouse was inoculated subcutaneously with 1×10⁵ KPL-4 cells, and mice were injected intravenously with (IV) PBMC 5×10⁶/each when the tumor grew to about 150 mm³. Mice were then grouped according to tumor volume and injected with (IV) drug (5 mg/kg) twice a week (BIW) in a volume of 0.1 mL/10 g body weight.

The effect of the drug on tumor growth was monitored specifically as T/C % or tumor growth inhibition rate (TGI %). The tumor diameter was measured twice a week with vernier calipers, and the tumor volume (V) was calculated by the following formula:

V=½×a×b²

Wherein a and b denote length and width, respectively.

T/C (%)=(T−T₀)/(C−C₀)×100

Wherein T and C are the tumor volumes at the end of the experiments; To and C₀ are the tumor volumes at the beginning of the experiment.

Tumor growth inhibition rate % (TGI %)=100−T/C (%)

When the tumor appears to regress,

Tumor growth inhibition rate % (TGI %)=100−(T−T_0)/T_0×100

Partial tumor regression (PR) was defined if the tumor volume decreased compared with its starting volume, i.e., when T<TO or C<C0; complete tumor regression (CR) was defined if the tumor disappeared completely.

### Experimental Results

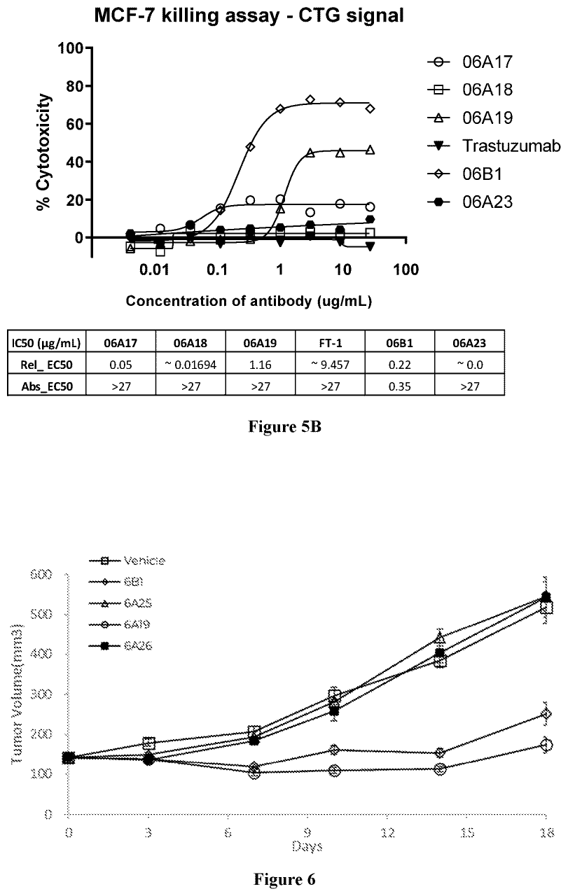

The results were shown in FIG. 6, which indicated that 6B1 and 6A19 (5 mg/kg, IV, BIW, 5 times in total) significantly inhibited the growth of subcutaneous transplanted tumors of human breast cancer KPL-4 cells in human PBMC immune-reconstituted mice, with tumor inhibition rates of 71% and 92%, respectively. It was observed that 2/6 mice in the 6A19 group had partial tumor regression; 6A25 and 6A26 (5 mg/kg, IV, BIW, 5 times in total) had no significant efficacy on the growth of subcutaneous transplanted tumors of KPL-4 in human PBMC immune-reconstituted mice. The aforementioned drugs were well tolerated by the tumor-bearing humanized immune-reconstituted mice, and no significant weight loss or other symptoms occurred during the administration.

3.3.2 Animal Model of BT474 with High Her2 Expression Level

### Experimental Protocols and Methods

6-8 weeks old female NCG mice were used, and each mouse was inoculated with 1×10⁷ BT474 cells and 1×10⁷ PBMC. When the tumor grew to ~150 mm³, mice were grouped according to tumor volume and injected with (IV) drug (5 mg/kg) twice a week (BIW) in a volume of 0.1 mL/10 g body weight.

The weight of the mice and the size of the tumors were measured twice a week. Tumor volume was calculated by the following formula:

Tumor volume (mm³)=½×(a×b²) (wherein a denotes the long diameter and b denotes the short diameter)

In the experiment, data was collected by using StudyDirector™ (Version No.: 3.1.399.19, Provider: Studylog System, Inc.), including the measurements of tumor short and long diameters and animal weights. The raw data was measured by the balance and vernier calipers and imported directly into the software. Any changes to the data would be recorded in this software.

### Experimental Results

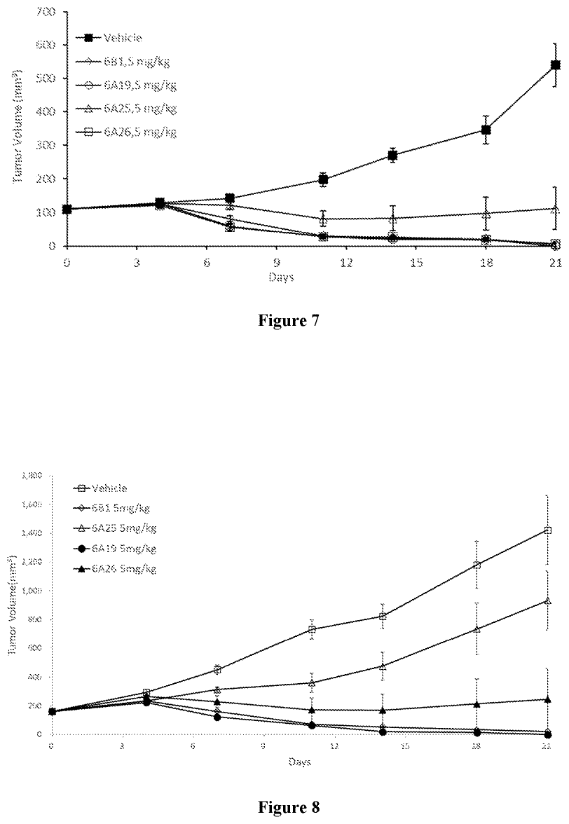

The results were shown in FIG. 7, which indicated that 6B1, 6A19, 6A25 and 6A26 (5 mg/kg, IV, BIW, two weeks in total) significantly inhibited the growth of subcutaneous transplanted tumors of human breast cancer BT474 cells in human PBMC immune-reconstituted mice, with tumor inhibition rates of 99.05%, 100.00%, 79.28% and 98.83%, respectively. The tumors partially regressed in 4/5, 5/5, 2/5 and 4/5 mice in each group, respectively. The aforementioned drugs were well tolerated by the tumor-bearing mice with reconstituted human immune system, and no significant weight loss or other symptoms occurred during the administration.

3.3.3 Animal Model of HT55 with Moderate Her2 Expression Level

### Experimental Protocols and Methods

5-7 weeks old female NCG mice were used and inoculated subcutaneously with HT55 cells (human colon cancer cell line, 5×10⁶/mouse) followed by intraperitoneal injection of PBMC cell (1×10⁷/mouse) suspension. When the tumor volume reached 160 mm³, group dosing of mice was initiated.

Tumor volume was measured three times a week by using vernier calipers to measure the long and short diameters of the tumors. Tumor volume reduction of 50% or more was recorded as PR (Partial Response); complete disappearance of tumor was recorded as CR (Complete Response).

### Experimental Results

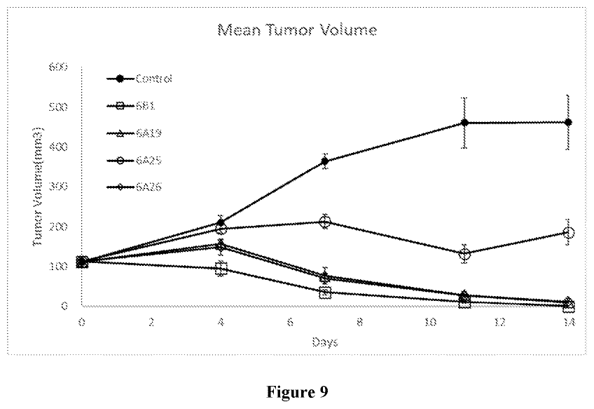

One animal in each of the 6B1 and 6A19 groups died during dosing due to the development of severe GVHD (graft-versus-host disease). Other results were shown in FIG. 8, which indicated that 6B1, 6A19, 6A25 and 6A26 (5 mg/kg, IV, BIW, two weeks in total) significantly inhibited the growth of subcutaneous transplanted tumors of human breast cancer HT55 cells in human PBMC immune-reconstituted mice, with tumor inhibition rates of 98.54%, 100.00%, 34.49% and 82.66%, respectively. The tumors partially regressed in 3/5, 5/5, 0/6 and 2/6 mice in each group, respectively. The aforementioned drugs were well tolerated by the tumor-bearing mice with reconstituted human immune system, and no significant weight loss or other symptoms occurred during the administration. The negative control in FIG. 8 is PBS.

3.3.4 Animal Model of PC3 with Low Expression Level

### Experimental Protocols and Methods

Female NCG mice aged 5-7 weeks were subcutaneously inoculated with PC-3 cell line (3×10⁶/mouse) and then intraperitoneally injected with PBMC cells (1.5×10⁶/mouse) suspension. Drugs were administrated for each group when the tumor grew to 60-80 m³.

### Test Indicators:

The tumor volume was measured three times a week, and the long diameter and short diameter of the tumor were measured with vernier caliper. The reduction of tumor volume by 50% or more was recorded as PR (partial response); and completely disappeared tumor was recorded as CR (complete response).

### Experimental Results

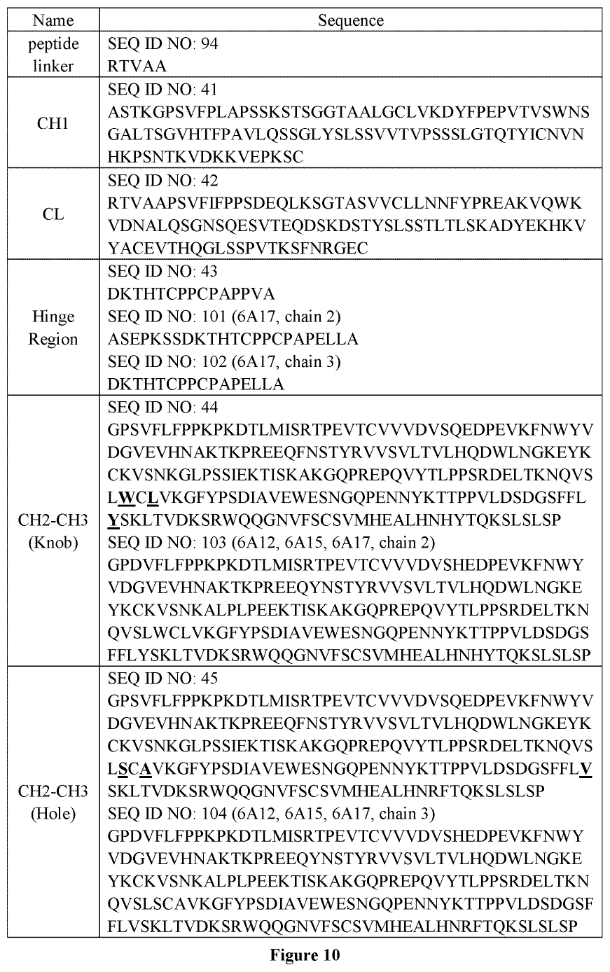

Results were shown in FIG. 9, which indicated that 6B1 can significantly inhibit the growth of PC-3 tumor on PC3. 6A19 has the similar effect as 6A26, which is stronger than 6A25, but slightly weaker than 6B1. The control in FIG. 9 is PBS.

What is claimed is:

1. An engineered antibody, comprising:
(i) a first polypeptide comprises a first heavy chain variable domain (VH1) linked to a second light chain variable domain (VL2);
(ii) a second polypeptide comprises a second heavy chain variable domain (VH2) linked to a first light chain variable domain (VL1);
(iii) a third polypeptide comprises a third heavy chain variable domain (VH3); and
(iv) a fourth polypeptide comprises a fourth light chain variable domain (VL3);
wherein,
the VL1 and the VH1 associate to form a first domain capable of binding to a first target;
the VL2 and the VH2 associate to form a second domain capable of binding to a second target;
the VL3 and the VH3 associate to form a third domain capable of binding to a third target; and
the second and the third polypeptide chain further comprises a first dimerization domain and a second dimerization domain, respectively, that are associated to form a dimer;
wherein one of the first target and the second target is an immune stimulatory target; and
the other two targets are different epitopes on a same tumor antigen that is capable of dimerization upon binding to a native ligand.

2. The engineered antibody of claim 1, wherein
the N-terminus of said VL1 is linked to C-terminus of said VH2, and N-terminus of said VL2 is covalently linked to C-terminus of said VH1, or
the N-terminus of said VH1 is linked to C-terminus of said VL2, and N-terminus of said VH2 is linked to C-terminus of said VL1.

3. The engineered antibody of claim 1, wherein the immune stimulatory target is selected from NKG2D, CD3 and CD16.

4. The engineered antibody of claim 3, wherein the immune stimulatory target is NKG2D.

5. The engineered antibody of claim 4, wherein the domain capable of binding to NKG2D comprises
(a) a VL comprising an LCDR1 comprising a sequence as shown in SEQ ID NO: 29, an LCDR2 comprising a sequence as shown in SEQ ID NO: 30, an LCDR3 comprising a sequence as shown in SEQ ID NO: 31, and a VH comprising an HCDR1 comprising a sequence as shown in SEQ ID NO: 25, an HCDR2 comprising a sequence as shown in SEQ ID NO: 26, and an HCDR3 comprising a sequence as shown in SEQ ID NO: 27; or
(b) a VL comprising an LCDR1 comprising a sequence as shown in SEQ ID NO: 118, an LCDR2 comprising a sequence as shown in SEQ ID NO: 119, an LCDR3 comprising a sequence as shown in SEQ ID NO: 120, and a VH comprising an HCDR1 comprising a sequence as shown in SEQ ID NO: 114, an HCDR2 comprising a sequence as shown in SEQ ID NO: 115, and an HCDR3 comprising a sequence as shown in SEQ ID NO: 116.

6. The engineered antibody of claim 4, wherein
(a) the VL comprises a sequence of SEQ ID NO: 32, and the VH comprises a sequence of SEQ ID NO: 28; or
(b) the VL comprises a sequence of SEQ ID NO: 121, and the VH comprises a sequence of SEQ ID NO: 117.

7. The engineered antibody of claim 3, wherein the immune stimulatory target is CD3.

8. The engineered antibody of claim 7, wherein the domain capable of binding to CD3 comprises a VL comprising an LCDR1 comprising a sequence as shown in SEQ ID NO: 5, an LCDR2 comprising a sequence as shown in SEQ ID NO: 6, an LCDR3 comprising a sequence as shown in SEQ ID NO: 7, and a VH comprising an HCDR1 comprising a sequence as shown in SEQ ID NO: 1, an HCDR2 comprising a sequence as shown in SEQ ID NO: 2, and an HCDR3 comprising a sequence as shown in SEQ ID NO: 3.

9. The engineered antibody of claim 7, wherein the VL comprises a sequence of SEQ ID NO: 8, and the VH comprises a sequence of SEQ ID NO: 4.

10. The engineered antibody of claim 3, wherein the immune stimulatory target is CD16.

11. The engineered antibody of claim 10, wherein the domain capable of binding to CD16 comprises a VL comprising an LCDR1 comprising a sequence as shown in SEQ ID NO: 37, an LCDR2 comprising a sequence as shown in SEQ ID NO: 38, an LCDR3 comprising a sequence as shown in SEQ ID NO: 39, and a VH comprising an HCDR1 comprising a sequence as shown in SEQ ID NO: 33, an HCDR2 comprising a sequence as shown in SEQ ID NO: 34, and an HCDR3 comprising a sequence as shown in SEQ ID NO: 35.

12. The engineered antibody of claim 10, wherein the VL comprises a sequence of SEQ ID NO: 40, and the VH comprises a sequence of SEQ ID NO: 36.

13. The engineered antibody of claim 1, wherein the tumor antigen is HER2.

14. The engineered antibody of claim 1, wherein
(a) one of the domain capable of binding to HER2 is a first HER2 binding domain, which is derived from the antigen-binding domain of Trastuzumab, and
the other domain capable of binding to HER2 is a second HER2 binding domain, which is derived from the antigen-binding domain of Pertuzumab;
(b) the VL2 and the VH2 associate to form the first HER2 binding domain, and the VL3 and the VH3 associate to form the second HER2 binding domain;
(c) the VL2 and the VH2 associate to form the second HER2 binding domain, and the VL3 and the VH3 associate to form the first HER2 binding domain;
(d) the VL1 and the VH1 associate to form the first HER2 binding domain, and the VL3 and the VH3 associate to form the second HER2 binding domain; or
(e) the VL1 and the VH1 associate to form the first HER2 binding domain, and the VL3 and the VH3 associate to form the second HER2 binding domain.

15. The engineered antibody of claim 14, wherein the first HER2 binding domain comprises an LCDR1 comprising a sequence as shown in SEQ ID NO: 13, an LCDR2 comprising a sequence as shown in SEQ ID NO: 14, an LCDR3 comprising a sequence as shown in SEQ ID NO: 15 or SEQ ID NO: 97, and the VH2 comprises an HCDR1 comprising a sequence as shown in SEQ ID NO: 9, an HCDR2 comprising a sequence as shown in SEQ ID NO: 10, and an HCDR3 comprising a sequence as shown in SEQ ID NO: 11.

16. The engineered antibody of claim 14 wherein the second HER2 binding domain comprises an LCDR1 comprising a sequence as shown in SEQ ID NO: 21, an LCDR2 comprising a sequence as shown in SEQ ID NO: 22, an LCDR3 comprising a sequence as shown in SEQ ID NO: 23, and the VH3 comprises an HCDR1 comprising a sequence as shown in SEQ ID NO: 17, an HCDR2 comprising a sequence as shown in SEQ ID NO: 18, and an HCDR3 comprising a sequence as shown in SEQ ID NO: 19.

17. The engineered antibody of any one of the preceding claims, wherein the VL1 and the VH1 are associated with a first non-native disulfide bond.

18. The engineered antibody of claim 17, wherein the first non-native disulfide bond is formed between two non-native cysteine residues in the VL1 and the VH1, respectively, optionally, the two non-native cysteine residues are Q100C in the VL1 and G44C in the VH1, wherein numbering is according to the Kabat index.

19. The engineered antibody of claim 18, wherein the VL2 and the VH2 are associated with either a native disulfide bond or a different non-native disulfide bond formed at positions distinct from those of the first non-native disulfide bond.

20. The engineered antibody of any one of claims 1-16, wherein the VL2 and the VH2 are associated with a second non-native disulfide bond.

21. The engineered antibody of claim 20, wherein the second non-native disulfide bond is formed between two non-native cysteine residues in the VL2 and the VH2, respectively, optionally, the two non-native cysteine residues are Q100C in the VL2 and G44C in the VH2, wherein numbering is according to the Kabat index.

22. The engineered antibody of claim 21, wherein the VL1 and the VH1 are associated with either a native disulfide bond or a different non-native disulfide bond formed at positions distinct from those of the second non-native disulfide bond.

23. The engineered antibody of any one of claims 17-19, wherein the VL1 and the VH1 are further associated with electrostatic interactions between two oppositely charged residues.

24. The engineered antibody of any one of claims 20-22, wherein the VL2 and the VH2 are further associated with electrostatic interactions between two oppositely charged residues.

25. The engineered antibody of claim 23 or 24, wherein
(a) the two oppositely charged residues are introduced to replace Q38 in the VL1 and Q39 in the VH1, respectively; or to replace Q38 in the VL2 and Q39 VH2, respectively;
(b) the two oppositely charged residues are introduced to replace Q40 in the VL1 and Q39 in the VH1, respectively;
(c) the two oppositely charged residues are introduced to replace Q37 in the VL1 and Q39 in the VH1, respectively; or
(d) the two oppositely charged residues are introduced to replace Q37 in the VL1 and Q39 in the VH1, respectively;
wherein numbering is according to the Kabat index.

26. The engineered antibody of any one of claims 23-25, wherein the two oppositely charged residues comprises a negatively charged amino acid residue selected from aspartic acid (D) or glutamic acid (E), and a positively charged amino acid residue selected from lysine (K), histidine (H) or arginine (R).

27. The engineered antibody of claim 26, wherein the two oppositely charged residues comprise Q38D in the VL1 and Q39K in the VH1, respectively; or Q38D in the VL2 and Q39K in the VH2, respectively, wherein numbering is according to the Kabat index.

28. The engineered antibody of any one of claims 19-22 and 24-27, wherein:
a) the VH2 comprises a sequence as shown in SEQ ID NO: 90 and the VL2 comprises a sequence as shown in SEQ ID NO: 91, and the VH3 comprises a sequence as shown in SEQ ID NO: 20 and the VL3 comprises a sequence as shown in SEQ ID NO: 24; or
b) the VH2 comprises a sequence as shown in SEQ ID NO: 90 and the VL2 comprises a sequence as shown in SEQ ID NO: 91; and the VH3 comprises a sequence as shown in SEQ ID NO: 106 and the VL3 comprises a sequence as shown in SEQ ID NO: 107.

29. The engineered antibody of any one of claims 19-22 and 24-27, wherein:
a) the VH2 comprises a sequence as shown in SEQ ID NO: 92 and a VL2 comprising a sequence as shown in SEQ ID NO: 93; and
b) the VH3 comprises a sequence as shown in SEQ ID NO: 12 and a VL3 comprising a sequence as shown in SEQ ID NO: 16.

30. The engineered antibody of any one of claims 19-22 and 24-27, wherein:
a) the VH2 comprises a sequence as shown in SEQ ID NO: 90 and a VL2 comprising a sequence as shown in SEQ ID NO: 91 or 98; and
b) the VH3 comprises a sequence as shown in SEQ ID NO: 12 and a VL3 comprising a sequence as shown in SEQ ID NO: 16 or 95.

31. The engineered antibody of any one of the preceding claims, wherein
the VL1 and the VH1 associate to form the first domain capable of binding to NKG2D,
the VL2 and the VH2 associate to form the first HER2 binding domain, and
the VL3 and the VH3 associate to form the second HER2 binding domain.

32. The engineered antibody of claim 31, wherein
(i) the VL1 comprises a sequence of SEQ ID NO: 32, and the VH1 comprises a sequence of SEQ ID NO: 28;
(ii) the VL2 comprises a sequence of SEQ ID NO: 91, and the VH2 comprises a sequence of SEQ ID NO: 90; and
(iii) the VL3 comprises a sequence of SEQ ID NO: 24, and the VH3 comprises a sequence of SEQ ID NO: 20.

33. The engineered antibody of any one of the preceding claims, wherein
the VL1 and the VH1 associate to form the first domain capable of binding to CD3,
the VL2 and the VH2 associate to form the second HER2 binding domain, and
the VL3 and the VH3 associate to form the first HER2 binding domain.

34. The engineered antibody of claim 33, wherein
(i) the VL1 comprises a sequence of SEQ ID NO: 8, and the VH1 comprises a sequence of SEQ ID NO: 4;
(ii) the VL2 comprises a sequence of SEQ ID NO: 93, and the VH2 comprises a sequence of SEQ ID NO: 92; and
(iii) the VL3 comprises a sequence of SEQ ID NO: 16, and the VH3 comprises a sequence of SEQ ID NO: 12.

35. The engineered antibody of any one of the preceding claims, wherein
the VL1 and the VH1 associate to form the first domain capable of binding to CD16,
the VL2 and the VH2 associate to form the second HER2 binding domain, and
the VL3 and the VH3 associate to form the first HER2 binding domain.

36. The engineered antibody of claim 35, wherein
(i) the VL1 comprises a sequence of SEQ ID NO: 40, and the VH1 comprises a sequence of SEQ ID NO: 36;
(ii) the VL2 comprises a sequence of SEQ ID NO: 93, and the VH2 comprises a sequence of SEQ ID NO: 92; and
(iii) the VL3 comprises a sequence of SEQ ID NO: 16, and the VH3 comprises a sequence of SEQ ID NO: 12.

37. The engineered antibody of any one of the preceding claims, wherein
the VL1 and the VH1 associate to form the first domain capable of binding to CD3,
the VL2 and the VH2 associate to form a third HER2 binding domain, and
the VL3 and the VH3 associate to form a fourth HER2 binding domain.

38. The engineered antibody of claim 37, wherein the third HER binding domain is derived from the antigen-binding domain of Trastuzumab and further modified to have reduced affinity to HER2.

39. The engineered antibody of claim 37 or 38, wherein
(i) the VL1 comprises a sequence of SEQ ID NO: 100, and the VH1 comprises a sequence of SEQ ID NO: 99;
(ii) the VL2 comprises a sequence of SEQ ID NO: 98, and the VH2 comprises a sequence of SEQ ID NO: 90; and
(iii) the VL3 comprises a sequence of SEQ ID NO: 95, and the VH3 comprises a sequence of SEQ ID NO: 12.

40. The engineered antibody of any one of the preceding claims, wherein the VL1 is linked to the VH2 via a first peptide linker, and wherein the VL2 is linked to the VH1 via a second peptide linker.

41. The engineered antibody of claim 40, wherein the first peptide linker and the second peptide linker each independently comprises 5 to 9 amino acids.

42. The engineered antibody of claim 41, wherein the first peptide linker and the second peptide linker each comprises a sequence as shown in SEQ ID NO: 94.

43. The engineered antibody of any one of the preceding claims, wherein the first dimerization domain and the second dimerization domain comprise CH3 domain of IgG, optionally further comprising one or more mutations that facilitate heterodimerization.

44. The engineered antibody of claim 43, wherein the first dimerization domain comprises a first mutation, and the second dimerization domain comprises a second mutation.

45. The engineered antibody of claim 44, wherein
a) the first mutation comprises T389W and/or S375C, and the second mutation comprises Y438V, T389S, L391A, and/or Y370C;
b) the first mutation comprises D427K and/or D377K, and the second mutation comprises K420D, and/or K440D;
c) the first mutation comprises D377K, E378K, and/or D427K, and the second mutation comprises K393E, K440D, and/or K470E;
d) the first mutation comprises S387H, and/or F436A, and the second mutation comprises Y370T, and/or T422F;
e) the first mutation comprises S387H, and/or T422F, and the second mutation comprises Y422T, and/or F436A;
f) the first mutation comprises K393D, and/or K440D, and the second mutation comprises E378K, and/or D427K; or
g) the first mutation comprises L372D, and/or L391E, and the second mutation comprises L372K, or T389K,
wherein numbering is according to the Kabat index.

46. The engineered antibody of claim 45, wherein the first mutation comprises T389W and the second domain comprises T389S, L391A and Y438V,
wherein numbering is according to the Kabat index.

47. The engineered antibody of any one of the preceding claims, wherein the first dimerization domain and/or the second dimerization domain further comprise CH2 domain and/or hinge region of IgG.

48. The engineered antibody of any one of the preceding claims, wherein the hinge region comprises a sequence as shown in SEQ ID NO: 43, 101 or 102.

49. The engineered antibody of any one of the preceding claims, wherein the third polypeptide further comprises CH1 region.

50. The engineered antibody of claim 49, wherein the CH1 region comprises a sequence as shown in SEQ ID NO: 41.

51. The engineered antibody of any one of the preceding claims, wherein the fourth polypeptide further comprises CL region.

52. The engineered antibody of claim 51, wherein the CL region comprises a sequence as shown in SEQ ID NO: 42.

53. The engineered antibody of any one of the preceding claims, wherein
(a) one of the first dimerization domain and the second dimerization domain comprises a sequence as shown in SEQ ID NO: 44, and the other comprises a sequence as shown in SEQ ID NO: 45; or
(b) one of the first dimerization domain and the second dimerization domain comprises a sequence as shown in SEQ ID NO: 103, and the other comprises a sequence as shown in SEQ ID NO: or 104.

54. The engineered antibody of claim 31, wherein the first polypeptide comprises a sequence as shown in SEQ ID NO: 58, the second polypeptide comprises a sequence as shown in SEQ ID NO: 59, the third polypeptide comprises a sequence as shown in SEQ ID NO: 20, and the fourth polypeptide comprises a sequence as shown in SEQ ID NO: 24.

55. The engineered antibody of claim 33, wherein the first polypeptide comprises a sequence as shown in SEQ ID NO: 62, the second polypeptide comprises a sequence as shown in SEQ ID NO: 63, the third polypeptide comprises a sequence as shown in SEQ ID NO: 12, and the fourth polypeptide comprises a sequence as shown in SEQ ID NO: 16.

56. The engineered antibody of claim 35, wherein the first polypeptide comprises a sequence as shown in SEQ ID NO: 74, the second polypeptide comprises a sequence as shown in SEQ ID NO: 75, the third polypeptide comprises a sequence as shown in SEQ ID NO: 12, and the fourth polypeptide comprises a sequence as shown in SEQ ID NO: 16.

57. The engineered antibody of claim 37, wherein the first polypeptide comprises a sequence as shown in SEQ ID NO: 78, the second polypeptide comprises a sequence as shown in SEQ ID NO: 79, the third polypeptide comprises a sequence as shown in SEQ ID NO: 80, and the fourth polypeptide comprises a sequence as shown in SEQ ID NO: 81.

58. The engineered antibody of any one of the preceding claims, which is linked to one or more conjugate moieties.

59. The engineered antibody of any one of the preceding claims, wherein the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

60. An isolated polynucleotide encoding the engineered antibody of any one of claims 1-59.

61. A vector comprising the isolated polynucleotide of claim 60.

62. A host cell comprising the vector of claim 61.

63. A pharmaceutical composition, comprising:
(i) the engineered antibody of any one of claims 1-59, or the polynucleotide encoding the engineered antibody of any one of claims 1-59; and
(ii) one or more pharmaceutically acceptable carriers, diluent, buffer or excipient.

64. The pharmaceutical composition of claim 63, further comprising an additional therapeutic agent.

65. The pharmaceutical composition of claim 64, wherein the additional therapeutic agent is an agent for treating a HER2-related disease or disorder.

66. The pharmaceutical composition of claim 65, wherein the HER2-related disease or disorder is a cancer, which is selected from the group consisting of: breast cancer, prostate cancer, lung cancer, bladder cancer, ovarian cancer, gastric cancer, colorectal cancer, esophageal cancer, head & neck cancer, cervical cancer, pancreatic cancer, testis cancer, urothelial cancer, endometrial cancer, malignant melanoma, and a soft-tissue cancer (e.g., synovial sarcoma).

67. A method of expressing the engineered antibody of any one of claims 1-59, comprising culturing the host cell of claim 62 under the condition at which the vector of claim 61 is expressed.

68. A method of treating, preventing or alleviating a disease or disorder in a subject, comprising administering to the subject a therapeutically effective amount of the engineered antibody of any one of claim 1-59.

69. A method of treating, preventing or alleviating a disease or disorder in a subject, comprising administering to the subject a therapeutically effective amount of the engineered antibody of any one of claims 1-70, or the polynucleotide encoding the engineered antibody of any one of claims 1-59, and/or the pharmaceutical composition of any one of claim 63-66.

70. The method of any one of claims 67-69, wherein the disease or disorder is a HER2-related disease or disorder.

71. The method of any one of claims 67-69, wherein the subject is human.

72. The method of any one of claims 67-69, wherein the administration is via oral, nasal, intravenous, subcutaneous, sublingual, or intramuscular administration.

73. Use of the engineered antibody of any one of claims 1-59, the pharmaceutical composition of any one of claims 63-66, and/or the polynucleotide encoding the engineered antibody of any one of claims 1-59 in the manufacture of a medicament for treating, preventing or alleviating a disease or disorder.
